(19) United States
(12) Patent Application Publication
(10) Pub. No.: US 2022/0378910 A1
(43) Pub. Date: Dec. 1, 2022

(54) METHODS OF INDUCING NEOEPITOPE-SPECIFIC T CELLS WITH A PD-1 AXIS BINDING ANTAGONIST AND AN RNA VACCINE

(71) Applicants: Genentech, Inc., South San Francisco, CA (US); BioNTech SE, Mainz (DE); Hoffmann-La Roche Inc., Little Falls, NJ (US)

(72) Inventors: Lars MUELLER, South San Francisco, CA (US); Rachel Lubong SABADO, South San Francisco, CA (US); Mahesh YADAV, South San Francisco, CA (US); Jingbin ZHANG, Mississauga (CA); Ugur SAHIN, Mainz (DE)

(73) Assignees: Genentech, Inc., South San Francisco, CA (US); BioNTech SE, Mainz (DE); Hoffmann-La Roche Inc., Little Falls, NJ (US)

(21) Appl. No.: 17/854,649

(22) Filed: Jun. 30, 2022

Related U.S. Application Data
(63) Continuation of application No. PCT/US2021/015710, filed on Jan. 29, 2021.
(60) Provisional application No. 63/041,707, filed on Jun. 19, 2020.
(60) Provisional application No. 62/968,818, filed on Jan. 31, 2020.

(51) Int. Cl. A61K 39/395; A61K 39/00; A61K 9/127; A61P 35/00; C07K 14/74; C07K 14/47; C07H 21/04; C07H 21/02

(52) U.S. Cl. CPC A61K 39/3955; A61K 39/001111; A61K 9/127; A61P 35/00; C07K 14/70539; C07K 14/4748; C07H 21/04; C07H 21/02; A61K 2039/53; C07K 2319/02; C07K 2319/03

(57) ABSTRACT

The present disclosure provides methods for inducing neoepitope-specific CD8+ T cells in an individual or for inducing trafficking of neoepitope-specific CD8+ T cells to a tumor in an individual using an RNA vaccine or using an RNA vaccine in combination with a PD-1 axis binding antagonist. Also provided herein are PD-1 axis binding antagonists and RNA vaccines that include one or more polynucleotides encoding one or more neoepitopes resulting from cancer-specific somatic mutations present in a tumor specimen obtained from the individual for use in methods of inducing neoepitope-specific CD8+ T cells in an individual or for inducing trafficking of neoepitope-specific CD8+ T cells to a tumor in an individual.

### CROSS-REFERENCE TO RELATED APPLICATION

This application claims the benefit of U.S. Provisional Application 63/041,707, filed Jun. 19, 2020, and U.S. Provisional Application 62/968,818, filed Jan. 31, 2020, each of which is hereby incorporated by reference in its entirety.

### FIELD

The present disclosure relates to methods for inducing a neoepitope-specific immune response in an individual with a tumor.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 146392050140SEQLIST.TXT, date recorded: Jan. 22, 2021, size: 41 KB).

### BACKGROUND

Modulating immune inhibitory pathways has been a major recent breakthrough in cancer treatment. Checkpoint blockade antibodies targeting cytotoxic T-lymphocyte antigen 4 (CTLA-4, YERVOY/ipilimumab), programmed cell-death protein 1 (PD-1, OPDIVO/nivolumab or KEYTRUDA/pembrolizumab), and PD-L1 (atezolizumab) have demonstrated acceptable toxicity, promising clinical responses, durable disease control, and improved survival in patients of various tumor indications. However, only a minority of patients experience durable responses to immune checkpoint blockade (ICB) therapy, and the remainder of patients show primary or secondary resistance.

Tumors characteristically harbor a remarkable number of somatic mutations. In turn, expression of a peptide containing a mutation may be recognized as a non-self neoepitope by the adaptive immune system. Upon recognition of a non-self antigen, cytotoxic T cells will trigger an immune response resulting in apoptosis of cells displaying the non-self neoepitope. Accordingly, therapeutic vaccines targeting immunogenic epitopes to activate the immune system are being developed and investigated for use in cancer therapy. However, thus far, therapeutic vaccines, while promising, have historically fallen short of expectations. One of the potential reasons is that cancer-specific T cells become functionally exhausted during chronic exposure to cancer cells.

Thus, combination therapy regimens employing two or more targeted cancer immunotherapy (CIT) agents, e.g., an immune checkpoint inhibitor and a therapeutic vaccine targeting immunogenic epitopes, may be required to fully engage the anti-tumor potential of the host immune system.

Accordingly, there is a need in the art for improved methods of inducing the anti-tumor immune responses of the host immune system.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

Provided herein are methods, kits, and uses involving a PD-1 axis binding antagonist (e.g., an anti-PD1 or anti-PD-L1 antibody) and an RNA vaccine for treating cancer.

In one aspect, provided herein is a method of inducing neoepitope-specific CD8+ T cells in an individual with a tumor, including administering to the individual an effective amount of an RNA vaccine, wherein the RNA vaccine includes one or more polynucleotides encoding one or more neoepitopes resulting from cancer-specific somatic mutations present in a tumor specimen obtained from the individual, and wherein about 1% to about 6% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine. In some embodiments, the peripheral blood sample includes about 5% or about 6% CD8+ T cells that are specific for at least one of the neoepitopes encoded by the one or more polynucleotides of the RNA vaccine. In some embodiments, the neoepitope-specific CD8+ T cells are detected in the peripheral blood sample by ex vivo ELISPOT or MHC multimer analysis. In some embodiments, administration of the RNA vaccine to the individual results in an induction of neoepitope-specific CD4+ T cells in the peripheral blood of the individual compared to prior to administration of the RNA vaccine, wherein the neoepitope-specific CD4+ T cells are specific for at least one of the neoepitopes encoded by the one or more polynucleotides of the RNA vaccine. In some embodiments, the neoepitope-specific CD4+ T cells are detected in a peripheral blood sample obtained from the individual by ex vivo ELISPOT analysis. In some embodiments, administration of the RNA vaccine to a plurality of individuals results in an induction of neoepitope-specific CD4+ or CD8+ T cells in the peripheral blood of at least about 70% of the individuals in the plurality compared to prior to administration of the RNA vaccine, wherein the neoepitope-specific CD4+ or CD8+ T cells are specific for at least one of the neoepitopes encoded by the one or more polynucleotides of the RNA vaccine, and wherein the induction of neoepitope-specific CD4+ or CD8+ T cells is assessed by ex vivo ELISPOT or MHC multimer analysis. In some embodiments, administration of the RNA vaccine to the individual results in an increase in the level of one or more inflammatory cytokines in the peripheral blood of the individual compared to the level of the one or more inflammatory cytokines prior to administration of the RNA vaccine. In some embodiments, the increase in the level of the one or more inflammatory cytokines is present in the peripheral blood of the individual at between about 4 to about 6 hours after administration of the RNA vaccine. In some embodiments, the one or more inflammatory cytokines are selected from IFNγ, IFNα, IL-12, or IL-6.

In another aspect, provided herein is a method of inducing neoepitope-specific CD8+ T cells in an individual with a tumor, including administering to the individual an effective amount of an RNA vaccine, wherein the RNA vaccine includes one or more polynucleotides encoding one or more neoepitopes resulting from cancer-specific somatic mutations present in a tumor specimen obtained from the individual, and wherein at least about 1% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine.

In another aspect, provided herein is a method of inducing trafficking of neoepitope-specific CD8+ T cells to a tumor in an individual, including administering to the individual an effective amount of an RNA vaccine, wherein the RNA vaccine includes one or more polynucleotides encoding one or more neoepitopes resulting from cancer-specific somatic mutations present in a tumor specimen obtained from the individual, and wherein the neoepitope-specific CD8+ T cells trafficked to the tumor after administration of the RNA vaccine are specific for at least one of the neoepitopes encoded by the one or more polynucleotides of the RNA vaccine.

In some embodiments, which may be combined with any of the preceding embodiments, the neoepitope-specific CD8+ T cells have a memory phenotype. In some embodiments, the neoepitope-specific CD8+ T cells having a memory phenotype are effector memory T cells (Tₑₘ). In some embodiments, the effector memory T cells (Tₑₘ) are CD45RO positive and CCR7 negative. In some embodiments, the neoepitope-specific CD8+ T cells are PD-1+.

In some embodiments, the individual has a tumor with a low to intermediate mutational burden. In some embodiments, the individual has a low tumor burden.

In some embodiments, which may be combined with any of the preceding embodiments, the tumor has low or negative PD-L1 expression. In some embodiments, less than 5% of tumor cells in a sample obtained from the tumor express PD-L1. In some embodiments, less than 5% of immune cells in a sample obtained from the tumor express PD-L1. In some embodiments, the percentage of tumor cells or immune cells in a sample obtained from the tumor that express PD-L1 is determined using immunohistochemistry.

In some embodiments, which may be combined with any of the preceding embodiments, administration of the RNA vaccine results in a complete response (CR) or partial response (PR) in the individual.

In some embodiments, which may be combined with any of the preceding embodiments, the individual has a locally advanced or metastatic solid tumor or has one or more metastatic relapses. In some embodiments, the tumor is a non-small cell lung (NSCLC), bladder, renal, head and neck, sarcoma, breast, melanoma, prostate, ovarian, gastric, liver, urothelial, colon, kidney, cervix, Merkel cell (MCC), endometrial, soft tissue sarcoma, esophageal, esophagogastric junction, bone sarcoma, thyroid, or colorectal tumor. In some embodiments, the breast tumor is a triple-negative breast (TNBC) tumor.

In some embodiments, the tumor is a urothelial tumor, and administration of the RNA vaccine to a plurality of individuals results in an objective response in at least about 10% of the individuals in the plurality. In some embodiments, the tumor is a renal tumor, and administration of the RNA vaccine to a plurality of individuals results in an objective response in at least about 22% of the individuals in the plurality. In some embodiments, the tumor is a melanoma tumor, and administration of the RNA vaccine to a plurality of individuals results in an objective response in at least about 30% of the individuals in the plurality. In some embodiments, the tumor is a TNBC tumor, and administration of the RNA vaccine to a plurality of individuals results in an objective response in at least about 4% of the individuals in the plurality. In some embodiments, the tumor is an NSCLC tumor, and administration of the RNA vaccine to a plurality of individuals results in an objective response in at least about 10% of the individuals in the plurality.

In some embodiments, the tumor is a urothelial tumor not previously treated with a checkpoint inhibitor, and administration of the RNA vaccine to a plurality of individuals results in an objective response in at least about 10% of the individuals in the plurality. In some embodiments, the tumor is a renal tumor not previously treated with a checkpoint inhibitor, and administration of the RNA vaccine to a plurality of individuals results in an objective response in at least about 22% of the individuals in the plurality. In some embodiments, the tumor is a melanoma tumor not previously treated with a checkpoint inhibitor, and administration of the RNA vaccine to a plurality of individuals results in an objective response in at least about 30% of the individuals in the plurality. In some embodiments, the tumor is a TNBC tumor not previously treated with a checkpoint inhibitor, and administration of the RNA vaccine to a plurality of individuals results in an objective response in at least about 4% of the individuals in the plurality. In some embodiments, the tumor is an NSCLC tumor not previously treated with a checkpoint inhibitor, and administration of the RNA vaccine to a plurality of individuals results in an objective response in at least about 10% of the individuals in the plurality.

In some embodiments, which may be combined with any of the preceding embodiments, prior to administration of the RNA vaccine, the individual has been treated with one or more cancer therapies or between 3 and 5 cancer therapies. In some embodiments, prior to administration of the RNA vaccine, the individual has been treated with a checkpoint inhibitor therapy. In some embodiments, prior to administration of the RNA vaccine, the individual has not been treated with a checkpoint inhibitor therapy. In some embodiments, prior to administration of the RNA vaccine, the individual has been treated with between about 1 to about 17 or between about 1 to about 9 prior systemic cancer therapies.

In some embodiments, which may be combined with any of the preceding embodiments, the RNA vaccine includes one or more polynucleotides encoding 10-20 neoepitopes resulting from cancer-specific somatic mutations present in the tumor specimen.

In some embodiments, which may be combined with any of the preceding embodiments, the RNA vaccine is formulated in a lipoplex nanoparticle or liposome. In some embodiments, the lipoplex nanoparticle or liposome includes one or more lipids that form a multilamellar structure that encapsulates the RNA of the RNA vaccine. In some embodiments, the one or more lipids includes at least one cationic lipid and at least one helper lipid. In some embodiments, the one or more lipids includes (R)-N,N,N-trimethyl-2,3-dioleyloxy-1-propanaminium chloride (DOTMA) and 1,2-dioleoyl-sn-glycero-3-phosphoethanolamine (DOPE). In some embodiments, at physiological pH the overall charge ratio of positive charges to negative charges of the liposome is 1.3:2 (0.65).

In some embodiments, which may be combined with any of the preceding embodiments, the RNA vaccine is administered to the individual at a dose of about 15 μg, about 25 μg, about 38 μg, about 50 μg, about 75 μg, or about 100 μg. In some embodiments, the RNA vaccine is administered intravenously to the individual.

In some embodiments, which may be combined with any of the preceding embodiments, the RNA vaccine is administered to the individual at an interval of 7 days or 1 week. In some embodiments, the RNA vaccine is administered to the individual at an interval of 14 days or 2 weeks. In some embodiments, the RNA vaccine is administered to the individual for 12 weeks or 84 days.

In some embodiments, which may be combined with any of the preceding embodiments, the RNA vaccine is administered to the individual in four 21-day Cycles, wherein the RNA vaccine is administered to the individual on Days 1, 8, and 15 of Cycle 1; Days 1, 8, and 15 of Cycle 2; Days 1 and 15 of Cycle 3; and Day 1 of Cycle 4.

In some embodiments, which may be combined with any of the preceding embodiments, the RNA vaccine is administered to the individual in 21-day Cycles, wherein the RNA vaccine is administered to the individual on Days 1, 8, and 15 of Cycle 1; Days 1, 8, and 15 of Cycle 2; Days 1 and 15 of Cycle 3; and Day 1 of Cycle 7. In some embodiments, the methods provided herein further include administering the RNA vaccine on Day 1 of Cycle 13, and every 24 weeks or 168 days thereafter. In some embodiments, administration of the RNA vaccine continues until an occurrence of disease progression in the individual.

In some embodiments, which may be combined with any of the preceding embodiments, the RNA vaccine is administered to the individual in 21-day Cycles, wherein the RNA vaccine is administered to the individual on Days 1, 8, and 15 of Cycle 2; Days 1 and 15 of Cycle 3; and Day 1 of Cycle 7. In some embodiments, the methods provided herein further include administering the RNA vaccine on Day 1 of Cycle 13, and every 24 weeks or 168 days thereafter. In some embodiments, administration of the RNA vaccine continues until an occurrence of disease progression in the individual.

In some embodiments, the RNA vaccine is administered to the individual in an induction stage and a maintenance stage after the induction stage, wherein the RNA vaccine is administered to the individual during the induction stage at an interval of 1 or 2 weeks, and wherein the RNA vaccine is administered to the individual during the maintenance stage at an interval of 24 weeks. In some embodiments, the RNA vaccine is administered to the individual in an induction stage and a maintenance stage after the induction stage, wherein the RNA vaccine is administered to the individual during the induction stage at an interval of 7 or 14 days, and wherein the RNA vaccine is administered to the individual during the maintenance stage at an interval of 168 days. In some embodiments, the RNA vaccine is administered to the individual in an induction stage and a maintenance stage after the induction stage, wherein the RNA vaccine is administered to the individual during the induction stage in four 21-day Cycles, wherein the RNA vaccine is administered to the individual during the induction stage on Days 1, 8, and 15 of Cycle 1; Days 1, 8, and 15 of Cycle 2; Days 1 and 15 of Cycle 3; and Day 1 of Cycle 4; and wherein the RNA vaccine is administered to the individual during the maintenance stage on Day 1 of Cycle 5 and once every 24 weeks or 168 days thereafter. In some embodiments, the induction stage includes up to 9 administrations of the RNA vaccine.

In some embodiments, which may be combined with any of the preceding embodiments, the RNA vaccine is administered to the individual in an induction stage and a maintenance stage after the induction stage, wherein the RNA vaccine is administered to the individual in 21-day Cycles; wherein, during the induction stage, the RNA vaccine is administered to the individual on Days 1, 8, and 15 of Cycle 1; Days 1, 8, and 15 of Cycle 2; Days 1 and 15 of Cycle 3; and Day 1 of Cycle 7; and wherein, during the maintenance stage, the RNA vaccine is administered to the individual on Day 1 of Cycle 13 and once every 24 weeks or 168 days thereafter. In some embodiments, the induction stage includes up to 9 administrations of the RNA vaccine. In some embodiments, the maintenance stage continues until an occurrence of disease progression in the individual.

In some embodiments, which may be combined with any of the preceding embodiments, the RNA vaccine is administered to the individual in an induction stage and a maintenance stage after the induction stage, wherein the RNA vaccine is administered to the individual in 21-day Cycles; wherein, during the induction stage, the RNA vaccine is administered to the individual on Days 1, 8, and 15 of Cycle 2; Days 1 and 15 of Cycle 3; and Day 1 of Cycle 7; and wherein, during the maintenance stage, the RNA vaccine is administered to the individual on Day 1 of Cycle 13 and once every 24 weeks or 168 days thereafter. In some embodiments, the induction stage includes 6 doses of the RNA vaccine. In some embodiments, the maintenance stage continues until an occurrence of disease progression in the individual.

In some embodiments, which may be combined with any of the preceding embodiments, the RNA vaccine includes an RNA molecule including, in the 5′→3′ direction: (1) a 5′ cap; (2) a 5′ untranslated region (UTR); (3) a polynucleotide sequence encoding a secretory signal peptide; (4) a polynucleotide sequence encoding the one or more neoepitopes resulting from cancer-specific somatic mutations present in the tumor specimen; (5) a polynucleotide sequence encoding at least a portion of a transmembrane and cytoplasmic domain of a major histocompatibility complex (MHC) molecule; (6) a 3′ UTR including: (a) a 3′ untranslated region of an Amino-Terminal Enhancer of Split (AES) mRNA or a fragment thereof; and (b) non-coding RNA of a mitochondrially encoded 12S RNA or a fragment thereof; and (7) a poly(A) sequence. In some embodiments, the RNA molecule further includes a polynucleotide sequence encoding an amino acid linker; wherein the polynucleotide sequences encoding the amino acid linker and a first of the one or more neoepitopes form a first linker-neoepitope module; and wherein the polynucleotide sequences forming the first linker-neoepitope module are between the polynucleotide sequence encoding the secretory signal peptide and the polynucleotide sequence encoding the at least portion of the transmembrane and cytoplasmic domain of the MHC molecule in the 5′→3′ direction. In some embodiments, the amino acid linker includes the sequence GGSGGGGSGG (SEQ ID NO:39). In some embodiments, the polynucleotide sequence encoding the amino acid linker includes the sequence

(SEQ ID NO: 37)
GGCGGCUCUGGAGGAGGCGGCUCCGGAGGC.

In some embodiments, which may be combined with any of the preceding embodiments, the RNA molecule further includes, in the 5′→3′ direction: at least a second linker-epitope module, wherein the at least second linker-epitope module includes a polynucleotide sequence encoding an amino acid linker and a polynucleotide sequence encoding a neoepitope; wherein the polynucleotide sequences forming the second linker-neoepitope module are between the polynucleotide sequence encoding the neoepitope of the first linker-neoepitope module and the polynucleotide sequence encoding the at least portion of the transmembrane and cytoplasmic domain of the MHC molecule in the 5′→3′ direction; and wherein the neoepitope of the first linker-epitope module is different from the neoepitope of the second linker-epitope module. In some embodiments, the RNA molecule includes 5 linker-epitope modules, and wherein the 5 linker-epitope modules each encode a different neoepitope. In some embodiments, the RNA molecule includes 10 linker-epitope modules, and wherein the 10 linker-epitope modules each encode a different neoepitope. In some embodiments, the RNA molecule includes 20 linker-epitope modules, and wherein the 20 linker-epitope modules each encode a different neoepitope.

In some embodiments, which may be combined with any of the preceding embodiments, the RNA molecule further includes a second polynucleotide sequence encoding an amino acid linker, wherein the second polynucleotide sequence encoding the amino acid linker is between the polynucleotide sequence encoding the neoepitope that is most distal in the 3′ direction and the polynucleotide sequence encoding the at least portion of the transmembrane and cytoplasmic domain of the MHC molecule.

In some embodiments, which may be combined with any of the preceding embodiments, the 5′ cap includes a D1 diastereoisomer of the structure:

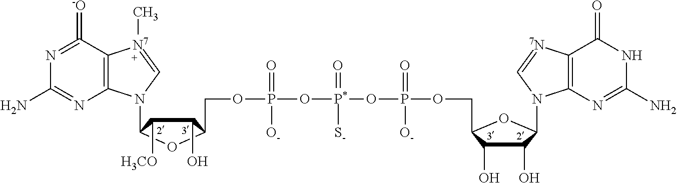

In some embodiments, which may be combined with any of the preceding embodiments, the 5′ UTR includes the sequence UUCUUCUGGUCCCCACAGACUCAGAGAGAACCCGCCACC (SEQ ID NO:23). In some embodiments, the 5′ UTR includes the sequence

(SEQ ID NO: 21)
GGCGAACUAGUAUUCUUCUGGUCCCCACAGACUCAGAGAGAACCCGCCA
CC.

In some embodiments, which may be combined with any of the preceding embodiments, the secretory signal peptide includes the amino acid sequence MRVMAPRTLILLLSGALALTETWAGS (SEQ ID NO:27). In some embodiments, the polynucleotide sequence encoding the secretory signal peptide includes the sequence

(SEQ ID NO: 25)
AUGAGAGUGAUGGCCCCCAGAACCCUGAUCCUGCUGCUGUCUGGCGCCCU
GGCCCUGACAGAGACAUGGGCCGGAAGC.

In some embodiments, which may be combined with any of the preceding embodiments, the at least portion of the transmembrane and cytoplasmic domain of the MHC molecule includes the amino acid sequence IVGIVAGLAVLAVVVIGAVVATVMCRRKSSGGKGGSYSQAASSDSAQGSDVSLTA (SEQ ID NO:30). In some embodiments, the polynucleotide sequence encoding the at least portion of the transmembrane and cytoplasmic domain of the MHC molecule includes the sequence

(SEQ ID NO: 28)
AUCGUGGGAAUUGUGGCAGGACUGGCAGUGCUGGCCGUGGUGGUGAUCG
GAGCCGUGGUGGCUACCGUGAUGUGCAGACGGAAGUCCAGCGGAGGCAA
GGGCGGCAGCUACAGCCAGGCCGCCAGCUCUGAUAGCGCCCAGGGCAGC
GACGUGUCACUGACAGCC.

In some embodiments, which may be combined with any of the preceding embodiments, the 3′ untranslated region of the AES mRNA includes the sequence CUGGUACUGCAUGCACGCAAUGCUAGCUGCCCCUUUCCCGUCCUGGGUACCCCGAGUC UCCCCCGACCUCGGGUCCCAGGUAUGCUCCCACCUCCACCUGCCCCACUCACCACCUCU GCUAGUUCCAGACACCUCC (SEQ ID NO:33). In some embodiments, the non-coding RNA of the mitochondrially encoded 12S RNA includes the sequence CAAGCACGCAGCAAUGCAGCUCAAAACGCUUAGCCUAGCCACACCCCCACGGGAAACA GCAGUGAUUAACCUUUAGCAAUAAACGAAAGUUUAACUAAGCUAUACUAACCCCAGG GUUGGUCAAUUUCGUGCCAGCCACACCG (SEQ ID NO:35). In some embodiments, the 3′ UTR includes the sequence CUCGAGCUGGUACUGCAUGCACGCAAUGCUAGCUGCCCCUUUCCCGUCCUGGGUACCC CGAGUCUCCCCCGACCUCGGGUCCCAGGUAUGCUCCCACCUCCACCUGCCCCACUCACC ACCUCUGCUAGUUCCAGACACCUCCCAAGCACGCAGCAAUGCAGCUCAAAACGCUUAG CCUAGCCACACCCCCACGGGAAACAGCAGUGAUUAACCUUUAGCAAUAAACGAAAGUU UAACUAAGCUAUACUAACCCCAGGGUUGGUCAAUUUCGUGCCAGCCACACCGAGACCU GGUCCAGAGUCGCUAGCCGCGUCGCU (SEQ ID NO:31). In some embodiments, the poly(A) sequence includes 120 adenine nucleotides.

In some embodiments, which may be combined with any of the preceding embodiments, the RNA vaccine includes an RNA molecule including, in the 5′→3′ direction: the polynucleotide sequence GGCGAACUAGUAUUCUUCUGGUCCCCACAGACUCAGAGAGAACCCGCCACCAUGAGAG UGAUGGCCCCCAGAACCCUGAUCCUGCUGCUGUCUGGCGCCCUGGCCCUGACAGAGAC AUGGGCCGGAAGC (SEQ ID NO:19); a polynucleotide sequence encoding the one or more neoepitopes resulting from cancer-specific somatic mutations present in the tumor specimen; and the polynucleotide sequence

(SEQ ID NO: 20)
AUCGUGGGAAUUGUGGCAGGACUGGCAGUGCUGGCCGUGGUGGUGAUCG
GAGCCGUGGUGGCUACCGUGAUGUGCAGACGGAAGUCCAGCGGAGGCAA
GGGCGGCAGCUACAGCCAGGCCGCCAGCUCUGAUAGCGCCCAGGGCAGC
GACGUGUCACUGACAGCCUAGUAACUCGAGCUGGUACUGCAUGCACGCA
AUGCUAGCUGCCCCUUUCCCGUCCUGGGUACCCCGAGUCUCCCCCGACC
UCGGGUCCCAGGUAUGCUCCCACCUCCACCUGCCCCACUCACCACCUCU
GCUAGUUCCAGACACCUCCCAAGCACGCAGCAAUGCAGCUCAAAACGCU
UAGCCUAGCCACACCCCCACGGGAAACAGCAGUGAUUAACCUUUAGCAA
UAAACGAAAGUUUAACUAAGCUAUACUAACCCCAGGGUUGGUCAAUUUC
GUGCCAGCCACACCGAGACCUGGUCCAGAGUCGCUAGCCGCGUCGCU.

In some embodiments, which may be combined with any of the preceding embodiments, the methods provided herein further include administering a PD-1 axis binding antagonist to the individual.

In some embodiments, which may be combined with any of the preceding embodiments, the PD-1 axis binding antagonist is a PD-1 binding antagonist. In some embodiments, the PD-1 binding antagonist is an anti-PD-1 antibody. In some embodiments, the anti-PD-1 antibody is nivolumab or pembrolizumab.

In some embodiments, which may be combined with any of the preceding embodiments, the PD-1 axis binding antagonist is a PD-L1 binding antagonist. In some embodiments, the PD-L1 binding antagonist is an anti-PD-L1 antibody. In some embodiments, the anti-PD-L1 antibody is avelumab or durvalumab. In some embodiments, the anti-PD-L1 antibody includes: (a) a heavy chain variable region (VH) that includes an HVR-H1 including an amino acid sequence of GFTFSDSWIH (SEQ ID NO:1), an HVR-2 including an amino acid sequence of AWISPYGGSTYYADSVKG (SEQ ID NO:2), and HVR-3 including an amino acid RHWPGGFDY (SEQ ID NO:3), and (b) a light chain variable region (VL) that includes an HVR-L1 including an amino acid sequence of RASQDVSTAVA (SEQ ID NO:4), an HVR-L2 including an amino acid sequence of SASFLYS (SEQ ID NO:5), and an HVR-L3 including an amino acid sequence of QQYLYHPAT (SEQ ID NO:6). In some embodiments, the anti-PD-L1 antibody includes a heavy chain variable region (V_{H}) including an amino acid sequence of SEQ ID NO:7 and a light chain variable region (V_{L}) including an amino acid sequence of SEQ ID NO:8. In some embodiments, the anti-PD-L1 antibody is atezolizumab.

In some embodiments, which may be combined with any of the preceding embodiments, the PD-1 axis binding antagonist is administered intravenously to the individual. In some embodiments, the anti-PD-L1 antibody is administered to the individual at a dose of about 1200 mg. In some embodiments, the PD-1 axis binding antagonist is administered to the individual at an interval of 21 days or 3 weeks.

In some embodiments, which may be combined with any of the preceding embodiments, the PD-1 axis binding antagonist is atezolizumab, and wherein atezolizumab is administered to the individual in 21-day cycles, wherein atezolizumab is administered on Day 1 of each of Cycles 1, 2, 3, 4, 5, 6, 7, 8, 9, 10, 11, and 12. In some embodiments, the methods provided herein further include administering atezolizumab on Day 1 of Cycle 13, and every 3 weeks or 21 days thereafter. In some embodiments, administration of atezolizumab continues until an occurrence of disease progression in the individual.

In some embodiments, which may be combined with any of the preceding embodiments, the PD-1 axis binding antagonist is atezolizumab, and atezolizumab is administered to the individual in 21-day cycles during an induction stage and during a maintenance stage after the induction stage; wherein, during the induction stage, atezolizumab is administered on Day 1 of each of Cycles 1, 2, 3, 4, 5, 6, 7, 8, 9, 10, 11, and 12; and wherein, during the maintenance stage after the induction stage, atezolizumab is administered on Day 1 of Cycle 13, and every 3 weeks or 21 days thereafter. In some embodiments, the maintenance stage continues until an occurrence of disease progression in the individual.

In some embodiments, which may be combined with any of the preceding embodiments, the individual is a human.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, and wherein about 1% to about 6% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine. In some embodiments, the method further includes administering a PD-1 axis binding antagonist to the individual.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, and wherein at least about 1% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine. In some embodiments, the method further includes administering a PD-1 axis binding antagonist to the individual.

In another aspect, provided herein is an RNA vaccine for use in a method of inducing trafficking of neoepitope-specific CD8+ T cells to a tumor in an individual, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, and wherein the neoepitope-specific CD8+ T cells trafficked to the tumor after administration of the RNA vaccine are specific for at least one of the neoepitopes encoded by the one or more polynucleotides of the RNA vaccine. In some embodiments, the method further includes administering a PD-1 axis binding antagonist to the individual.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, and wherein about 1% to about 6% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, and wherein at least about 1% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine.

In another aspect, provided herein is a PD-1 axis binding antagonist for use in a method of inducing trafficking of neoepitope-specific CD8+ T cells to a tumor in an individual, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, and wherein the neoepitope-specific CD8+ T cells trafficked to the tumor after administration of the PD-1 axis binding antagonist and the RNA vaccine are specific for at least one of the neoepitopes encoded by the one or more polynucleotides of the RNA vaccine.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein the neoepitope-specific CD8+ T cells are detected in the peripheral blood sample by ex vivo ELISPOT or MHC multimer analysis.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein the peripheral blood sample includes about 5% or about 6% CD8+ T cells that are specific for at least one of the neoepitopes encoded by the one or more polynucleotides of the RNA vaccine.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein administration of the RNA vaccine to the individual results in an induction of neoepitope-specific CD4+ T cells in the peripheral blood of the individual compared to prior to administration of the RNA vaccine, wherein the neoepitope-specific CD4+ T cells are specific for at least one of the neoepitopes encoded by the one or more polynucleotides of the RNA vaccine.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein administration of the RNA vaccine to a plurality of individuals results in an induction of neoepitope-specific CD4+ or CD8+ T cells in the peripheral blood of at least about 70% of the individuals in the plurality compared to prior to administration of the RNA vaccine, wherein the neoepitope-specific CD4+ or CD8+ T cells are specific for at least one of the neoepitopes encoded by the one or more polynucleotides of the RNA vaccine, and wherein the induction of neoepitope-specific CD4+ or CD8+ T cells is assessed by ex vivo ELISPOT or MHC multimer analysis.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein administration of the RNA vaccine to the individual results in an increase in the level of one or more inflammatory cytokines in the peripheral blood of the individual compared to the level of the one or more inflammatory cytokines prior to administration of the RNA vaccine. In some embodiments, the one or more inflammatory cytokines are selected from IFNγ, IFNα, IL-12, or IL-6.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein the neoepitope-specific CD8+ T cells are effector memory T cells (Tₑₘ).

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein the neoepitope-specific CD8+ T cells are PD-1+.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein administration of the RNA vaccine results in a complete response (CR) or partial response (PR) in the individual.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein the RNA vaccine is administered to the individual at a dose of about 15 μg, about 25 μg, about 38 μg, about 50 μg, about 75 μg, or about 100 μg.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein the RNA vaccine is administered to the individual at a dose of about 15 μg, about 25 μg, about 38 μg, about 50 μg, about 75 μg, or about 100 μg, wherein the RNA vaccine is administered to the individual in 21-day Cycles, wherein the RNA vaccine is administered to the individual on Days 1, 8, and 15 of Cycle 1; Days 1, 8, and 15 of Cycle 2; Days 1 and 15 of Cycle 3; and Day 1 of Cycle 7; and, optionally, on Day 1 of Cycle 13 and every 24 weeks or 168 days thereafter.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein the RNA vaccine is administered to the individual at a dose of about 15 μg, about 25 μg, about 38 μg, about 50 μg, about 75 μg, or about 100 μg, wherein the RNA vaccine is administered to the individual in an induction stage and a maintenance stage after the induction stage, wherein the RNA vaccine is administered to the individual in 21-day Cycles; wherein, during the induction stage, the RNA vaccine is administered to the individual on Days 1, 8, and 15 of Cycle 1; Days 1, 8, and 15 of Cycle 2; Days 1 and 15 of Cycle 3; and Day 1 of Cycle 7; and wherein, during the maintenance stage, the RNA vaccine is administered to the individual on Day 1 of Cycle 13 and once every 24 weeks or 168 days thereafter.

In another aspect, provided herein is an RNA vaccine for use in a method of inducing trafficking of neoepitope-specific CD8+ T cells to a tumor in an individual, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein the neoepitope-specific CD8+ T cells trafficked to the tumor after administration of the RNA vaccine are specific for at least one of the neoepitopes encoded by the one or more polynucleotides of the RNA vaccine, and wherein the neoepitope-specific CD8+ T cells are effector memory T cells (Tₑₘ).

In another aspect, provided herein is an RNA vaccine for use in a method of inducing trafficking of neoepitope-specific CD8+ T cells to a tumor in an individual, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein the neoepitope-specific CD8+ T cells trafficked to the tumor after administration of the RNA vaccine are specific for at least one of the neoepitopes encoded by the one or more polynucleotides of the RNA vaccine, and wherein the neoepitope-specific CD8+ T cells are PD-1+.

In another aspect, provided herein is an RNA vaccine for use in a method of inducing trafficking of neoepitope-specific CD8+ T cells to a tumor in an individual, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein the neoepitope-specific CD8+ T cells trafficked to the tumor after administration of the RNA vaccine are specific for at least one of the neoepitopes encoded by the one or more polynucleotides of the RNA vaccine, and wherein administration of the RNA vaccine results in a complete response (CR) or partial response (PR) in the individual.

In another aspect, provided herein is an RNA vaccine for use in a method of inducing trafficking of neoepitope-specific CD8+ T cells to a tumor in an individual, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein the neoepitope-specific CD8+ T cells trafficked to the tumor after administration of the RNA vaccine are specific for at least one of the neoepitopes encoded by the one or more polynucleotides of the RNA vaccine, and wherein the RNA vaccine is administered to the individual at a dose of about 15 μg, about 25 μg, about 38 μg, about 50 μg, about 75 μg, or about 100 μg.

In another aspect, provided herein is an RNA vaccine for use in a method of inducing trafficking of neoepitope-specific CD8+ T cells to a tumor in an individual, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein the neoepitope-specific CD8+ T cells trafficked to the tumor after administration of the RNA vaccine are specific for at least one of the neoepitopes encoded by the one or more polynucleotides of the RNA vaccine, and wherein the RNA vaccine is administered to the individual at a dose of about 15 μg, about 25 μg, about 38 μg, about 50 μg, about 75 μg, or about 100 μg, wherein the RNA vaccine is administered to the individual in 21-day Cycles, wherein the RNA vaccine is administered to the individual on Days 1, 8, and 15 of Cycle 1; Days 1, 8, and 15 of Cycle 2; Days 1 and 15 of Cycle 3; and Day 1 of Cycle 7; and, optionally, on Day 1 of Cycle 13 and every 24 weeks or 168 days thereafter.

In another aspect, provided herein is an RNA vaccine for use in a method of inducing trafficking of neoepitope-specific CD8+ T cells to a tumor in an individual, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein the neoepitope-specific CD8+ T cells trafficked to the tumor after administration of the RNA vaccine are specific for at least one of the neoepitopes encoded by the one or more polynucleotides of the RNA vaccine, and wherein the RNA vaccine is administered to the individual at a dose of about 15 μg, about 25 μg, about 38 μg, about 50 μg, about 75 μg, or about 100 μg, wherein the RNA vaccine is administered to the individual in an induction stage and a maintenance stage after the induction stage, wherein the RNA vaccine is administered to the individual in 21-day Cycles; wherein, during the induction stage, the RNA vaccine is administered to the individual on Days 1, 8, and 15 of Cycle 1; Days 1, 8, and 15 of Cycle 2; Days 1 and 15 of Cycle 3; and Day 1 of Cycle 7; and wherein, during the maintenance stage, the RNA vaccine is administered to the individual on Day 1 of Cycle 13 and once every 24 weeks or 168 days thereafter.

In some embodiments of any of the preceding aspects, the method further includes administering a PD-1 axis binding antagonist to the individual.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, and wherein the neoepitope-specific CD8+ T cells are detected in the peripheral blood sample by ex vivo ELISPOT or MHC multimer analysis.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, and wherein the peripheral blood sample includes about 5% or about 6% CD8+ T cells that are specific for at least one of the neoepitopes encoded by the one or more polynucleotides of the RNA vaccine.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, and wherein administration of the PD-1 axis binding antagonist and the RNA vaccine to the individual results in an induction of neoepitope-specific CD4+ T cells in the peripheral blood of the individual compared to prior to administration of the RNA vaccine, wherein the neoepitope-specific CD4+ T cells are specific for at least one of the neoepitopes encoded by the one or more polynucleotides of the RNA vaccine.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, and wherein administration of the PD-1 axis binding antagonist and the RNA vaccine to a plurality of individuals results in an induction of neoepitope-specific CD4+ or CD8+ T cells in the peripheral blood of at least about 70% of the individuals in the plurality compared to prior to administration of the RNA vaccine, wherein the neoepitope-specific CD4+ or CD8+ T cells are specific for at least one of the neoepitopes encoded by the one or more polynucleotides of the RNA vaccine, and wherein the induction of neoepitope-specific CD4+ or CD8+ T cells is assessed by ex vivo ELISPOT or MHC multimer analysis.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, and wherein administration of the PD-1 axis binding antagonist and the RNA vaccine to the individual results in an increase in the level of one or more inflammatory cytokines in the peripheral blood of the individual compared to the level of the one or more inflammatory cytokines prior to administration of the RNA vaccine. In some embodiments, the one or more inflammatory cytokines are selected from IFNγ, IFNα, IL-12, or IL-6.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, and wherein the neoepitope-specific CD8+ T cells are effector memory T cells (Tₑₘ).

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, and wherein the neoepitope-specific CD8+ T cells are PD-1+.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, and wherein administration of the PD-1 axis binding antagonist and the RNA vaccine results in a complete response (CR) or partial response (PR) in the individual.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, wherein the PD-1 axis binding antagonist is atezolizumab, wherein the atezolizumab is administered to the individual at a dose of about 1200 mg at an interval of 21 days or 3 weeks, and wherein the RNA vaccine is administered to the individual at a dose of about 15 μg, about 25 μg, about 38 μg, about 50 μg, about 75 μg, or about 100 μg in 21-day cycles.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, wherein the PD-1 axis binding antagonist is atezolizumab, wherein the atezolizumab is administered to the individual at a dose of about 1200 mg in 21-day cycles, wherein atezolizumab is administered on Day 1 of each of Cycles 1, 2, 3, 4, 5, 6, 7, 8, 9, 10, 11, and 12, and, optionally, on Day 1 of Cycle 13 and every 3 weeks or 21 days thereafter; and wherein the RNA vaccine is administered to the individual at a dose of about 15 μg, about 25 μg, about 38 μg, about 50 μg, about 75 μg, or about 100 μg, wherein the RNA vaccine is administered to the individual in 21-day Cycles, wherein the RNA vaccine is administered to the individual on Days 1, 8, and 15 of Cycle 1; Days 1, 8, and 15 of Cycle 2; Days 1 and 15 of Cycle 3; and Day 1 of Cycle 7; and, optionally, on Day 1 of Cycle 13, and every 24 weeks or 168 days thereafter.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein about 1% to about 6% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, wherein the PD-1 axis binding antagonist is atezolizumab, wherein the atezolizumab is administered to the individual at a dose of about 1200 mg in 21-day cycles during an induction stage and during a maintenance stage after the induction stage, wherein, during the induction stage, atezolizumab is administered on Day 1 of each of Cycles 1, 2, 3, 4, 5, 6, 7, 8, 9, 10, 11, and 12, and wherein, during the maintenance stage after the induction stage, atezolizumab is administered on Day 1 of Cycle 13, and every 3 weeks or 21 days thereafter; and wherein the RNA vaccine is administered to the individual at a dose of about 15 μg, about 25 μg, about 38 μg, about 50 μg, about 75 μg, or about 100 μg, wherein the RNA vaccine is administered to the individual in an induction stage and a maintenance stage after the induction stage, wherein the RNA vaccine is administered to the individual in 21-day Cycles; wherein, during the induction stage, the RNA vaccine is administered to the individual on Days 1, 8, and 15 of Cycle 1; Days 1, 8, and 15 of Cycle 2; Days 1 and 15 of Cycle 3; and Day 1 of Cycle 7; and wherein, during the maintenance stage, the RNA vaccine is administered to the individual on Day 1 of Cycle 13 and once every 24 weeks or 168 days thereafter.

In another aspect, provided herein is a PD-1 axis binding antagonist for use in a method of inducing trafficking of neoepitope-specific CD8+ T cells to a tumor in an individual, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein the neoepitope-specific CD8+ T cells trafficked to the tumor after administration of the PD-1 axis binding antagonist and the RNA vaccine are specific for at least one of the neoepitopes encoded by the one or more polynucleotides of the RNA vaccine, and wherein the neoepitope-specific CD8+ T cells are effector memory T cells (Tₑₘ).

In another aspect, provided herein is a PD-1 axis binding antagonist for use in a method of inducing trafficking of neoepitope-specific CD8+ T cells to a tumor in an individual, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein the neoepitope-specific CD8+ T cells trafficked to the tumor after administration of the PD-1 axis binding antagonist and the RNA vaccine are specific for at least one of the neoepitopes encoded by the one or more polynucleotides of the RNA vaccine, and wherein the neoepitope-specific CD8+ T cells are PD-1+.

In another aspect, provided herein is a PD-1 axis binding antagonist for use in a method of inducing trafficking of neoepitope-specific CD8+ T cells to a tumor in an individual, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein the neoepitope-specific CD8+ T cells trafficked to the tumor after administration of the PD-1 axis binding antagonist and the RNA vaccine are specific for at least one of the neoepitopes encoded by the one or more polynucleotides of the RNA vaccine, and wherein administration of the RNA vaccine results in a complete response (CR) or partial response (PR) in the individual.

In another aspect, provided herein is a PD-1 axis binding antagonist for use in a method of inducing trafficking of neoepitope-specific CD8+ T cells to a tumor in an individual, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein the neoepitope-specific CD8+ T cells trafficked to the tumor after administration of the PD-1 axis binding antagonist and the RNA vaccine are specific for at least one of the neoepitopes encoded by the one or more polynucleotides of the RNA vaccine, wherein the PD-1 axis binding antagonist is atezolizumab, wherein the atezolizumab is administered to the individual at a dose of about 1200 mg at an interval of 21 days or 3 weeks, and wherein the RNA vaccine is administered to the individual at a dose of about 15 μg, about 25 μg, about 38 μg, about 50 μg, about 75 μg, or about 100 μg in 21-day cycles.

In another aspect, provided herein is a PD-1 axis binding antagonist for use in a method of inducing trafficking of neoepitope-specific CD8+ T cells to a tumor in an individual, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein the neoepitope-specific CD8+ T cells trafficked to the tumor after administration of the PD-1 axis binding antagonist and the RNA vaccine are specific for at least one of the neoepitopes encoded by the one or more polynucleotides of the RNA vaccine, wherein the PD-1 axis binding antagonist is atezolizumab, wherein the atezolizumab is administered to the individual at a dose of about 1200 mg in 21-day cycles, wherein atezolizumab is administered on Day 1 of each of Cycles 1, 2, 3, 4, 5, 6, 7, 8, 9, 10, 11, and 12, and, optionally, on Day 1 of Cycle 13, and every 3 weeks or 21 days thereafter; and wherein the RNA vaccine is administered to the individual at a dose of about 15 μg, about 25 μg, about 38 μg, about 50 μg, about 75 μg, or about 100 μg, wherein the RNA vaccine is administered to the individual in 21-day Cycles, wherein the RNA vaccine is administered to the individual on Days 1, 8, and 15 of Cycle 1; Days 1, 8, and 15 of Cycle 2; Days 1 and 15 of Cycle 3; and Day 1 of Cycle 7; and, optionally, on Day 1 of Cycle 13, and every 24 weeks or 168 days thereafter.

In another aspect, provided herein is a PD-1 axis binding antagonist for use in a method of inducing trafficking of neoepitope-specific CD8+ T cells to a tumor in an individual, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein the neoepitope-specific CD8+ T cells trafficked to the tumor after administration of the PD-1 axis binding antagonist and the RNA vaccine are specific for at least one of the neoepitopes encoded by the one or more polynucleotides of the RNA vaccine, wherein the PD-1 axis binding antagonist is atezolizumab, wherein the atezolizumab is administered to the individual at a dose of about 1200 mg in 21-day cycles during an induction stage and during a maintenance stage after the induction stage, wherein, during the induction stage, atezolizumab is administered on Day 1 of each of Cycles 1, 2, 3, 4, 5, 6, 7, 8, 9, 10, 11, and 12, and wherein, during the maintenance stage after the induction stage, atezolizumab is administered on Day 1 of Cycle 13, and every 3 weeks or 21 days thereafter; and wherein the RNA vaccine is administered to the individual at a dose of about 15 μg, about 25 μg, about 38 μg, about 50 μg, about 75 μg, or about 100 μg, wherein the RNA vaccine is administered to the individual in an induction stage and a maintenance stage after the induction stage, wherein the RNA vaccine is administered to the individual in 21-day Cycles; wherein, during the induction stage, the RNA vaccine is administered to the individual on Days 1, 8, and 15 of Cycle 1; Days 1, 8, and 15 of Cycle 2; Days 1 and 15 of Cycle 3; and Day 1 of Cycle 7; and wherein, during the maintenance stage, the RNA vaccine is administered to the individual on Day 1 of Cycle 13 and once every 24 weeks or 168 days thereafter.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein the neoepitope-specific CD8+ T cells are detected in the peripheral blood sample by ex vivo ELISPOT or MHC multimer analysis.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein administration of the RNA vaccine to the individual results in an induction of neoepitope-specific CD4+ T cells in the peripheral blood of the individual compared to prior to administration of the RNA vaccine, wherein the neoepitope-specific CD4+ T cells are specific for at least one of the neoepitopes encoded by the one or more polynucleotides of the RNA vaccine.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein administration of the RNA vaccine to a plurality of individuals results in an induction of neoepitope-specific CD4+ or CD8+ T cells in the peripheral blood of at least about 70% of the individuals in the plurality compared to prior to administration of the RNA vaccine, wherein the neoepitope-specific CD4+ or CD8+ T cells are specific for at least one of the neoepitopes encoded by the one or more polynucleotides of the RNA vaccine, and wherein the induction of neoepitope-specific CD4+ or CD8+ T cells is assessed by ex vivo ELISPOT or MHC multimer analysis.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein administration of the RNA vaccine to the individual results in an increase in the level of one or more inflammatory cytokines in the peripheral blood of the individual compared to the level of the one or more inflammatory cytokines prior to administration of the RNA vaccine. In some embodiments, the one or more inflammatory cytokines are selected from IFNγ, IFNα, IL-12, or IL-6.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein the neoepitope-specific CD8+ T cells are effector memory T cells (Tₑₘ).

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein the neoepitope-specific CD8+ T cells are PD-1+.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein administration of the RNA vaccine results in a complete response (CR) or partial response (PR) in the individual.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein the RNA vaccine is administered to the individual at a dose of about 15 μg, about 25 μg, about 38 μg, about 50 μg, about 75 μg, or about 100 μg.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein the RNA vaccine is administered to the individual at a dose of about 15 μg, about 25 μg, about 38 μg, about 50 μg, about 75 μg, or about 100 μg, wherein the RNA vaccine is administered to the individual in 21-day Cycles, wherein the RNA vaccine is administered to the individual on Days 1, 8, and 15 of Cycle 1; Days 1, 8, and 15 of Cycle 2; Days 1 and 15 of Cycle 3; and Day 1 of Cycle 7; and, optionally, on Day 1 of Cycle 13 and every 24 weeks or 168 days thereafter.

In another aspect, provided herein is an RNA vaccine for use in a method of inducing neoepitope-specific CD8+ T cells in an individual with a tumor, said method including administering to the individual an effective amount of the RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine, and wherein the RNA vaccine is administered to the individual at a dose of about 15 μg, about 25 μg, about 38 μg, about 50 μg, about 75 μg, or about 100 μg, wherein the RNA vaccine is administered to the individual in an induction stage and a maintenance stage after the induction stage, wherein the RNA vaccine is administered to the individual in 21-day Cycles; wherein, during the induction stage, the RNA vaccine is administered to the individual on Days 1, 8, and 15 of Cycle 1; Days 1, 8, and 15 of Cycle 2; Days 1 and 15 of Cycle 3; and Day 1 of Cycle 7; and wherein, during the maintenance stage, the RNA vaccine is administered to the individual on Day 1 of Cycle 13 and once every 24 weeks or 168 days thereafter.

In some embodiments of any of the preceding aspects, the method further includes administering a PD-1 axis binding antagonist to the individual.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, and wherein the neoepitope-specific CD8+ T cells are detected in the peripheral blood sample by ex vivo ELISPOT or MHC multimer analysis.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, and wherein administration of the PD-1 axis binding antagonist and the RNA vaccine to the individual results in an induction of neoepitope-specific CD4+ T cells in the peripheral blood of the individual compared to prior to administration of the RNA vaccine, wherein the neoepitope-specific CD4+ T cells are specific for at least one of the neoepitopes encoded by the one or more polynucleotides of the RNA vaccine.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, and wherein administration of the PD-1 axis binding antagonist and the RNA vaccine to a plurality of individuals results in an induction of neoepitope-specific CD4+ or CD8+ T cells in the peripheral blood of at least about 70% of the individuals in the plurality compared to prior to administration of the RNA vaccine, wherein the neoepitope-specific CD4+ or CD8+ T cells are specific for at least one of the neoepitopes encoded by the one or more polynucleotides of the RNA vaccine, and wherein the induction of neoepitope-specific CD4+ or CD8+ T cells is assessed by ex vivo ELISPOT or MHC multimer analysis.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, and wherein administration of the PD-1 axis binding antagonist and the RNA vaccine to the individual results in an increase in the level of one or more inflammatory cytokines in the peripheral blood of the individual compared to the level of the one or more inflammatory cytokines prior to administration of the RNA vaccine. In some embodiments, the one or more inflammatory cytokines are selected from IFNγ, IFNα, IL-12, or IL-6.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, and wherein the neoepitope-specific CD8+ T cells are effector memory T cells (Tₑₘ).

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, and wherein the neoepitope-specific CD8+ T cells are PD-1+.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, and wherein administration of the PD-1 axis binding antagonist and the RNA vaccine results in a complete response (CR) or partial response (PR) in the individual.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, wherein the PD-1 axis binding antagonist is atezolizumab, wherein the atezolizumab is administered to the individual at a dose of about 1200 mg at an interval of 21 days or 3 weeks, and wherein the RNA vaccine is administered to the individual at a dose of about 15 μg, about 25 μg, about 38 μg, about 50 μg, about 75 μg, or about 100 μg in 21-day cycles.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, wherein the PD-1 axis binding antagonist is atezolizumab, wherein the atezolizumab is administered to the individual at a dose of about 1200 mg in 21-day cycles, wherein atezolizumab is administered on Day 1 of each of Cycles 1, 2, 3, 4, 5, 6, 7, 8, 9, 10, 11, and 12, and, optionally, on Day 1 of Cycle 13, and every 3 weeks or 21 days thereafter; and wherein the RNA vaccine is administered to the individual at a dose of about 15 μg, about 25 μg, about 38 μg, about 50 μg, about 75 μg, or about 100 μg, wherein the RNA vaccine is administered to the individual in 21-day Cycles, wherein the RNA vaccine is administered to the individual on Days 1, 8, and 15 of Cycle 1; Days 1, 8, and 15 of Cycle 2; Days 1 and 15 of Cycle 3; and Day 1 of Cycle 7; and, optionally, on Day 1 of Cycle 13, and every 24 weeks or 168 days thereafter.

In another aspect, provided herein is a PD-1 axis binding antagonist for use in a method of inducing neoepitope-specific CD8+ T cells in an individual with a tumor, said method including administering to the individual an effective amount of the PD-1 axis binding antagonist and an RNA vaccine, wherein the RNA vaccine includes one or more polynucleotides encoding one or more neoepitopes resulting from cancer-specific somatic mutations present in a tumor specimen obtained from the individual, wherein at least about 1% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine, wherein the PD-1 axis binding antagonist is atezolizumab, wherein the atezolizumab is administered to the individual at a dose of about 1200 mg in 21-day cycles during an induction stage and during a maintenance stage after the induction stage, wherein, during the induction stage, atezolizumab is administered on Day 1 of each of Cycles 1, 2, 3, 4, 5, 6, 7, 8, 9, 10, 11, and 12, and wherein, during the maintenance stage after the induction stage, atezolizumab is administered on Day 1 of Cycle 13, and every 3 weeks or 21 days thereafter; and wherein the RNA vaccine is administered to the individual at a dose of about 15 μg, about 25 μg, about 38 μg, about 50 μg, about 75 μg, or about 100 μg, wherein the RNA vaccine is administered to the individual in an induction stage and a maintenance stage after the induction stage, wherein the RNA vaccine is administered to the individual in 21-day Cycles; wherein, during the induction stage, the RNA vaccine is administered to the individual on Days 1, 8, and 15 of Cycle 1; Days 1, 8, and 15 of Cycle 2; Days 1 and 15 of Cycle 3; and Day 1 of Cycle 7; and wherein, during the maintenance stage, the RNA vaccine is administered to the individual on Day 1 of Cycle 13 and once every 24 weeks or 168 days thereafter.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

The patent or application file contains at least one drawing executed in color. Copies of this patent or patent application publication with color drawings will be provided by the office upon request and payment of the necessary fee.

FIG. 1 shows the general structure of an exemplary RNA vaccine (i.e., a poly-neoepitope RNA). This figure is a schematic illustration of the general structure of the RNA drug substance with constant 5′-cap (beta-S-ARCA (D1)), 5′- and 3′-untranslated regions (hAg-Kozak and FI, respectively), N- and C-terminal fusion tags (sec_{2.0} and MITD, respectively), and poly(A)-tail (A120) as well as tumor-specific sequences encoding the neoepitopes (neo1 to 10) fused by GS-rich linkers.

FIG. 2 is the ribonucleotide sequence (5′->3′) of the constant region of an exemplary RNA vaccine (SEQ ID NO: 42). The linkage between the first two G residues is the unusual bond (5′→5′)-ppₛp- as shown in FIG. 3 for the 5′ capping structure. The insertion site for patient cancer-specific sequences is between the C131 and A132 residues (marked in bold text). “N” refers to the position of polynucleotide sequence(s) encoding one or more (e.g., 1-20) neoepitopes (separated by optional linkers).

FIG. 3 is the 5′-capping structure beta-S-ARCA(D1) (m₂^{7-2′-O} GppₛpG) used at the 5′ end of the RNA constant regions. The stereogenic P center is Rp-configured in the “D1” isomer. Note: Shown in red are the differences between beta-S-ARCA(D1) and the basic cap structure m⁷GpppG; an —OCH3 group at the C2′ position of the building block m⁷G and substitution of a non-bridging oxygen at the beta-phosphate by sulphur. Owing to the presence of a stereogenic P center (labelled with *), the phosphorothioate cap analogue beta-S-ARCA exists as two diastereomers. Based on their elution order in reversed-phase high-performance liquid chromatography, these have been designated as 01 and 02.

FIG. 4 is a diagram of the design of the Phase Ia/Ib study described in Examples 1-5. Subjects in the Phase Ia dose escalation study were administered the RNA vaccine as a monotherapy at doses of 25 μg, 38 μg, 50 μg, 75 μg, or 100 μg. During initial treatment (induction stage), the RNA vaccine was administered on Days 1, 8, and 15 of Cycle 1, on Days 1, 8 and 15 of Cycle 2, on Days 1 and 15 of Cycle 3, and on Day 1 of Cycle 7 (each cycle was 21 days). During the maintenance stage after initial treatment, the RNA vaccine was administered on Day 1 of Cycle 13, and every 8 cycles thereafter (i.e., every 24 weeks thereafter, or every 168 days thereafter) until disease progression (PD) (each cycle was 21 days). Subjects in the Phase Ib study were administered the RNA vaccine in doses of 15 μg (not shown), 25 μg, 38 μg, or 50 μg in combination with 1200 mg atezolizumab. The Phase Ib study included a dose escalation phase for the RNA vaccine and an expansion phase in which patients with the indicated checkpoint inhibitor naïve or checkpoint inhibitor experienced tumor types were administered the RNA vaccine at a dose of 15 μg or 25 μg in combination with atezolizumab (additional tumor types in the Phase Ib expansion phase are provided in Example 1). During initial treatment (induction stage), atezolizumab was administered on Day 1 of each of Cycles 1-12; and the RNA vaccine was administered on Days 1, 8, and 15 of Cycle 1, on Days 1, 8 and 15 of Cycle 2, on Days 1 and 15 of Cycle 3, and on Day 1 of Cycle 7 (each cycle was 21 days). During the maintenance stage after initial treatment, atezolizumab was administered every 3 weeks until disease progression (PD), starting on Day 1 of Cycle 13; and the RNA vaccine was administered on Day 1 of Cycle 13, and every 8 cycles thereafter (i.e., every 24 weeks thereafter, or every 168 days thereafter) until disease progression (PD) (each cycle was 21 days).

FIGS. 5A-5C show the innate immune responses induced by the RNA vaccine administered as a monotherapy (Phase Ia) or in combination with atezolizumab (Phase Ib). FIG. 5A shows the levels of IFNg in the plasma (pg/ml) of patients administered 25 μg of the RNA vaccine in Phase Ia of the study. Each line represents a single patient. “C”=Cycle (i.e., C1=Cycle 1; C2=Cycle 2, etc.). “D”=Day (i.e., D1=Day 1, D8=Day 8, etc.). “hr”=hours after administration of a dose of RNA vaccine. The days on which the RNA vaccine was administered are indicated by the solid arrows.

FIG. 5B shows the median plasma IFNg levels at 4 hours after each administration of the RNA vaccine in patients administered the RNA vaccine at the indicated doses as a monotherapy (Phase Ia; Ph1a) or in combination with atezolizumab (Phase Ib; Ph1b). Each circle represents the median value for IFNg levels at 4 hours following all RNA vaccine doses for each individual patient. FIG. 5C shows the median value for IFNa plasma levels at 4 hours after each administration of the RNA vaccine in patients administered the RNA vaccine at the indicated doses as a monotherapy (Phase Ia; Ph1a) or in combination with atezolizumab (Phase Ib; Ph1b). Each circle represents the median value for IFNa levels at 4 hours following all RNA vaccine doses for each individual patient.

FIG. 6 provides a diagram of the ex vivo EliSpot assays used to evaluate neoantigen-specific CD4+ and CD8+ T cell immune responses following administration of the RNA vaccine as a monotherapy (Phase Ia) or in combination with atezolizumab (Phase Ib).

FIGS. 7A-7D show the results of EliSpot assays that evaluated neoantigen-specific immune responses following administration of the RNA vaccine as a monotherapy (Phase Ia) or in combination with atezolizumab (Phase Ib). FIG. 7A shows neoantigen-specific immune responses in patients administered the RNA vaccine as a monotherapy (Phase Ia) at Cycle 4, Day 1. FIG. 7B shows neoantigen-specific immune responses in patients administered the RNA vaccine in combination with atezolizumab (Phase Ib) at Cycle 4, Day 1. The asterisk indicates that the Cycle 1, Day 1 and Cycle 1, Day 8 doses of the RNA vaccine were 30 μg, followed by doses of 15 μg. In FIGS. 7A-7B, the y-axis shows the number of neoantigens tested in the EliSpot assays. The dark-colored bars and corresponding numbers represent the number of positive neoantigen hits identified in the EliSpot assays. The light-colored bars represent the number of negative neoantigen hits. The RNA vaccine dose is indicated. An EliSpot response was defined as >15 spots per 300,000 cells and statistically different from background wells (which are generally <10 spots); all neoantigens were tested in duplicate. Positive hits (“+ve hits”) refer to neoantigens that had an EliSpot assay response at Cycle 4, Day 1 and no EliSpot assay response at baseline. Negative hits (“no hit”) refer to neoantigens that had a negative EliSpot assay response at Cycle 4, Day 1. FIG. 7C shows the sum of IFNg forming spots for each neoantigen identified as a positive hit by EliSpot assay for patients in the Phase Ib study administered the RNA vaccine at the indicated doses. Each colored box represents the number of IFNg forming spots for an individual neoantigen. An EliSpot response was defined as >15 spots per 300,000 cells and statistically different from background wells (which are generally <10 spots); all neoantigens were tested in duplicate. FIG. 7D provides the average number of IFNg forming spots in patients in the Phase Ib study administered the RNA vaccine at the indicated doses. The middle line in the box plots indicates the median number of IFNg forming spots; the boxes show the interquartile ranges; the error bars show the minimum and maximum values.

FIG. 8 provides a diagram of the MHC multimer staining assays used to evaluate neoantigen-specific CD8+ T cell immune responses following administration of the RNA vaccine as a monotherapy (Phase Ia) or in combination with atezolizumab (Phase Ib).

FIGS. 9A-9G show the results of EliSpot assays and MHC multimer staining assays that evaluated neoantigen-specific immune responses in a CIT-naïve, triple-negative breast cancer patient administered the RNA vaccine at a dose of 25 μg in combination with atezolizumab (Phase Ib; patient 22). FIG. 9A shows the results of bulk PBMC EliSpot assays that evaluated neoantigen-specific immune responses in patient 22 at baseline and at Cycle 4, Day 1. The tested neoantigens and controls are on the x-axis; the y-axis shows the number of IFNg forming spots per 300,000 PBMCs. Neoantigens R3, and R8 are indicated in boxes. The horizontal dashed line indicates the threshold for determining a positive hit in the EliSpot assays. A positive hit was defined as >15 spots per 300,000 cells and statistically different from background wells (which are generally <10 spots). Neoantigens were tested in duplicate; CEFT=epitopes from Cytomegalovirus, Epstein-Barr virus, Influenza virus, and Tetanus toxin; CEF=epitopes from Cytomegalovirus, Epstein-Barr virus, and Influenza virus. FIG. 9B shows the R8 neoantigen-specific CD8+ T cell immune response in patient 22 at the indicated times assessed by MHC multimer staining assays. The scatter plots show CD8+ T cells stained with MHC multimer in two different configurations on the x- and y-axes. Double positive cells were labeled as neoantigen specific. The percent of neoantigen-specific CD8+ T cells is shown in the top right quadrant of the scatter plots. FIG. 9C shows an analysis of CD45RO and CCR7 expression in the neoantigen-specific CD8+ T cell population shown in FIG. 9B at Cycle 3, Day 1. As indicated in the legend on the right, CD8+ naïve cells are in the top left quadrant of the scatter plot; central memory T cells (Tcm) are in the top right quadrant of the scatter plot; CD45RA+ effector memory T cells (TEMRA) are in the bottom left quadrant of the scatter plot; and effector memory T cells (Tem) are in the bottom right quadrant of the scatter plot. FIG. 9D shows an analysis of PD-1 expression in the neoantigen-specific CD8+ T cell population shown in FIG. 9B at Cycle 3, Day 1.

FIG. 9E shows the R3 neoantigen-specific CD8+ T cell immune response in patient 22 at the indicated times assessed by MHC multimer staining assays. The scatter plots show CD8+ T cells stained with MHC multimer in two different configurations on the x- and y-axes. The percent of neoantigen-specific CD8+ T cells is shown in the top right quadrant of the scatter plots. FIG. 9F shows an analysis of CD45RO and CCR7 expression in the neoantigen-specific CD8+ T cell population shown in FIG. 9E at Cycle 3, Day 1. As indicated in the legend on the right, CD8+ naïve cells are in the top left quadrant of the scatter plot; central memory T cells (Tcm) are in the top right quadrant of the scatter plot; CD45RA+ effector memory T cells (TEMRA) are in the bottom left quadrant of the scatter plot; and effector memory T cells (Tem) are in the bottom right quadrant of the scatter plot. FIG. 9G shows an analysis of PD-1 expression in the neoantigen-specific CD8+ T cell population shown in FIG. 9E at Cycle 3, Day 1.

FIGS. 10A-10B provide an overview of the manufacturing work flow and proposed mechanisms of action for the RNA vaccine. FIG. 10A depicts the manufacturing process of the RNA vaccine. During manufacture, blood samples and tumor samples (e.g., tumor biopsies) are collected from the patient, and tumor DNA and non-tumor DNA (e.g., peripheral blood mononuclear cell DNA) is subjected to sequencing (e.g., next-generation sequencing and/or whole exome sequencing) to identify non-synonymous somatic mutations specifically present in the patient's tumor. RNA from the tumor sample is also subjected to sequencing to assess expression of proteins with identified non-synonymous somatic mutations. Neoantigens are predicted using a bioinformatics workflow that ranks their likely immunogenicity. A database that provides comprehensive information about expression levels of respective wild-type genes in healthy tissues is used for development of a personalized risk mitigation strategy by removing target candidates with an unfavorable risk profile. For example, mutations occurring in proteins with a possible higher auto-immunity risk in critical organs are filtered out and not considered for vaccine production. Up to 20 neoantigens that are predicted to elicit CD8⁺ T-cell and/or CD4⁺ T-cell responses for an individual patient are selected for inclusion into the vaccine. The RNA vaccine includes a 5′ cap, a 5′ untranslated region (UTR), an N-terminal fusion tag (e.g., SEC), up to 20 neoantigens (e.g., 2 decatopes) with linker sequences between each neoantigen, a C-terminal fusion tag (e.g., MITD), a 3′ UTR, and a poly(A) tail. The RNA vaccine is formulated, e.g., in a lipoplex. The RNA vaccine may be stored prior to intravenous administration to the patient. As depicted in FIG. 10A, the RNA vaccine is believed to function by stimulating an innate immune response (e.g., by acting as an intrinsic TLR7/8 agonist), and by stimulating expression of neoantigens and subsequent neoantigen presentation by antigen presenting cells. FIG. 10B depicts details of the proposed mechanism of action of the RNA vaccine. See also Kranz et al (2016) Nature, 16; 534(7607):396-401).

FIG. 11 provides a summary of adverse events that occurred in greater than 10% of patients in the Phase Ia study of the RNA vaccine monotherapy. The relative frequencies of all reported AEs and AEs related to study treatment are provided. The severity of the reported AEs is indicated in the legend on the right (Grades 1-5). ^{a}Serious adverse events (SAE) of malignant neoplasm progression were reported in 16% of patients (data not shown). Systemic reactions of infusion related reaction and cytokine release syndrome are indicated. ^{b}According to the National Cancer Institute (NCI) Common Terminology Criteria for Adverse Events (CTCAE) Version 5.0.

FIGS. 12A-12B show the levels of IFNγ in the plasma of patients administered the RNA vaccine as a monotherapy (Phase Ia) at a dose of 25 μg. FIG. 12A shows the levels of IFNγ (pg/ml) in the plasma of patients administered the RNA vaccine as a monotherapy at a dose of 25 μg at the indicated times. Each line represents a single patient. FIG. 12B provides a representative pattern of the levels of IFNγ (pg/ml) in the plasma of nine patients administered the RNA vaccine as a monotherapy at a dose of 25 μg at the indicated times. The RNA vaccine dosing regimen is shown below the plot in FIG. 12B. Each arrow represents administration of an RNA vaccine dose. “C”=Cycle (i.e., C1=Cycle 1; C2=Cycle 2, etc.); “D”=Day (i.e., D1=Day 1, D8=Day 8, etc.); “HR”=hours after administration of a dose of RNA vaccine.

FIG. 13 shows the levels of IL-6 and IFNα (pg/ml) in the plasma of patients administered the RNA vaccine as a monotherapy at a dose of 25 μg at the indicated times. Each line represents a single patient. “C”=Cycle (i.e., C1=Cycle 1; C2=Cycle 2, etc.); “D”=Day (i.e., D1=Day 1, D8=Day 8, etc.); “HR”=hours after administration of a dose of RNA vaccine.

FIGS. 14A-14B provide an overview of neoantigen-specific immune responses induced by administration of the RNA vaccine as a monotherapy (Phase Ia) in fourteen patients. FIG. 14A shows the number of patients in the Phase Ia study that had at least one neoantigen specific immune response determined by EliSpot and/or MHC multimer staining assays. FIG. 14B shows the numbers of neoantigens that exhibited a neoantigen immune response by ex vivo EliSpot assay in the indicated patients.

FIG. 15 shows the results of T cell receptor (TCR) sequencing experiments in a tumor of a prostate cancer patient treated with the RNA vaccine as a monotherapy at the dose of 75 μg. The y-axis shows the frequency of TCRs (Log₁₀) in the tumor prior to administration of the RNA vaccine (baseline). The x-axis shows the frequency of TCRs (Log₁₀) in the tumor after treatment with the RNA vaccine. RNA vaccine-specific TCRs are indicated with shaded circles and other TCRs are indicated by empty circles.

FIGS. 16A-16C show the results of MHC multimer staining assays that evaluated neoantigen-specific CD8+ T cell immune responses in a prostate cancer patient treated with the RNA vaccine as a monotherapy at a dose of 38 μg. FIG. 16A shows neoantigen-specific CD8+ T cell immune response at the indicated times. The scatter plots show CD8+ T cells stained with MHC multimer in two different configurations on the x- and y-axes. The percent of neoantigen-specific CD8+ T cells is shown in the top right quadrant of the scatter plots. “C”=Cycle (i.e., C1=Cycle 1; C2=Cycle 2, etc.); “D”=Day (i.e., D1=Day 1, D8=Day 8, etc.). FIG. 16B shows an analysis of CD45RO and CCR7 expression in the neoantigen-specific CD8+ T cell population shown in FIG. 16A at Cycle 4, Day 1. CD8+ naïve cells are in the top left quadrant of the scatter plot (Tn); central memory T cells (Tcm) are in the top right quadrant of the scatter plot; and effector memory T cells (Tem) are in the bottom right quadrant of the scatter plot. The percentage of Tem cells is indicated. FIG. 16C shows an analysis of PD-1 expression in the neoantigen-specific CD8+ T cell population shown in FIG. 16A at Cycle 4, Day 1. The percentage of PD-1+CD8+ T cells is indicated.

FIG. 17 provides a summary of clinical responses observed in patients treated with the RNA vaccine as a monotherapy. Each bar represents an individual patient, with the tumor type for each patient provided on the x-axis. The y-axis indicates the best change in the sum of longest diameter of target lesions (SLD) observed for each patient. The dose of RNA vaccine administered to each patient is indicated in the legend on the right and over each bar. Baseline PD-L1 expression on tumor infiltrating immune cells (IC) or tumor cells (TC) analyzed by SP142 Ventana assay is indicated below the graph for each patient (N=no; Y=yes). The best overall response (BOR) for each patient during study is indicated below the graph (PD=disease progression; SD=stable disease; CR=complete response). In addition, whether each patient received a prior treatment with a checkpoint inhibitor (“CPI Experienced”) is indicated below the graph (N=no; Y=yes). HNC=head and neck cancer; STS=soft tissue sarcoma; EGJ=esophagogastric junction. The horizontal dashed lines indicate the thresholds for disease progression and partial response according to the Response Evaluation Criteria in Solid Tumours (RECIST) criteria (i.e., ≥20% increase in SLD from baseline=disease progression (PD); and >30% decrease in SLD from baseline=partial response (PR)).

FIG. 18 shows neoantigen-specific immune responses measured by EliSpot assay at baseline and at Cycle 4, Day 1 in one gastric cancer patient that exhibited a complete response (CR) after treatment with the RNA vaccine as a monotherapy at a dose of 50 μg. Individual neoantigens and controls are indicated on the x-axis. The y-axis shows the IFNγ forming spots per 300,000 peripheral blood mononuclear cells (PBMCs). The horizontal dashed line indicates the threshold for determining a positive hit in the EliSpot assays. An EliSpot positive hit was defined as >15 spots per 300,000 cells and statistically different from background wells (which are generally <10 spots); all neoantigens were tested in duplicate. ^{a}Serious AEs (SAEs) of malignant neoplasm progression were reported in 14% of patients (data not shown).

FIG. 19 provides a summary of adverse events that occurred in greater than 10% of patients in the Phase Ib study of the RNA vaccine administered in combination with atezolizumab. The relative frequencies of all reported AEs and AEs related to study treatment are provided. The severity of the reported AEs is indicated in the legend on the right (Grades 1-5). Systemic reactions of infusion related reaction, cytokine release syndrome, and influenza-like illness are indicated.

FIG. 20 shows the number of patients in the Phase Ib study that had at least one neoantigen-specific immune response determined by EliSpot and/or MHC multimer staining assays.

FIG. 21 shows the results of T cell receptor (TCR) sequencing experiments in a tumor of a rectal cancer patient treated with atezolizumab and the RNA vaccine at a dose of 38 μg. The y-axis shows the frequency of TCRs (Log₁₀) in the tumor prior to administration of atezolizumab and the RNA vaccine (baseline). The x-axis shows the frequency of TCRs (Log₁₀) in the tumor after treatment with atezolizumab and the RNA vaccine. RNA vaccine-specific TCRs are indicated with shaded circles and other TCRs are indicated by empty circles.

FIG. 22 provides a summary of clinical responses observed in patients treated with the RNA vaccine in combination with atezolizumab. Each bar represents an individual patient, with the tumor type for each patient provided on the x-axis. The y-axis indicates the best change in the sum of longest diameters (SLD) observed for each patient. The dose of RNA vaccine administered to each patient is indicated in the legend on the right and above each bar. ^{a}Baseline PD-L1 expression on tumor infiltrating immune cells (IC) or tumor cells (TC) analyzed by SP142 Ventana assay for each patient is indicated below the graph (N=no; Y=yes). The best overall response (BOR) for each patient during study is indicated below the graph (PD=disease progression; SD=stable disease; PR=partial response; CR=complete response). In addition, whether each patient received a prior treatment with a checkpoint inhibitor (“CPI Experienced”) is indicated below the graph (N=no; Y=yes). HNC=head and neck cancer; STS=soft tissue sarcoma; NSCLC=non-small cell lung cancer; MCC=Merkel cell carcinoma. The box indicates a CPI-experienced patient with triple negative breast cancer (TNBC) that was administered the RNA vaccine at a dose of 38 μg in combination with atezolizumab. The horizontal dashed lines indicate the thresholds for disease progression and partial response according to the Response Evaluation Criteria in Solid Tumours (RECIST) criteria (i.e., ≥20% increase in SLD from baseline=disease progression (PD); and ≥30% decrease in SLD from baseline=partial response (PR)).

FIGS. 23A-23B show tumor and neoantigen-specific immune responses observed in a triple negative breast cancer (TNBC) patient that was administered the RNA vaccine at a dose of 38 μg in combination with atezolizumab (indicated by the box in FIG. 22). As shown in FIG. 22, this TNBC patient exhibited a partial response to treatment, had baseline PD-L1 expression on ≥5% of tumor infiltrating immune cells or tumor cells (assessed by SP142 Ventana assay), and had been previously treated with a checkpoint inhibitor (CPI experienced). The computerized tomography (CT) scan images provided in FIG. 23A show that the patient had several tumor masses associated with metastatic disease at screening, and that tumors were reduced at Cycle 4 of treatment (tumors are indicated by the arrows). FIG. 23B shows that the patient was negative for neoantigen-specific CD8+ T cells at screening (0.01%; background levels), and that the levels of neoantigen-specific CD8+ T cells increased to 2.2% at Cycle 4 of treatment (as assessed by MHC multimer staining) The scatter plots show CD8+ T cells stained with MHC multimer in two different configurations on the x- and y-axes.

FIGS. 24A-24E provide the change overtime in the sums of longest diameters (SLDs) and objective response rates (ORRs) for checkpoint inhibitor naïve patients in the indication-specific expansion phase of the Phase Ib study described herein. FIG. 24A shows the change overtime in SLD and the ORR for checkpoint inhibitor naïve urothelial carcinoma (UC) patients. FIG. 24B shows the change overtime in SLD and the ORR for checkpoint inhibitor naïve renal cell carcinoma (RCC) patients. FIG. 24C shows the change overtime in SLD and the ORR for checkpoint inhibitor naïve melanoma patients. FIG. 24D shows the change overtime in SLD and the ORR for checkpoint inhibitor naïve triple negative breast cancer (TNBC) patients. FIG. 24E shows the change overtime in SLD and the ORR for checkpoint inhibitor naïve non-small cell lung cancer (NSCLC) patients. The arrows indicate patients that continue on active treatment. In FIGS. 24A-24E, the horizontal dashed lines indicate the thresholds for disease progression and partial response according to the Response Evaluation Criteria in Solid Tumours (RECIST) criteria (i.e., ≥20% increase in SLD from baseline=disease progression (PD); and ≥30% decrease in SLD from baseline=partial response (PR)).

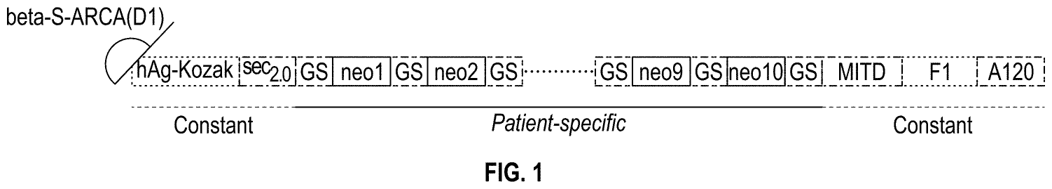

### DETAILED DESCRIPTION

### I. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The term “PD-1 axis binding antagonist” refers to a molecule that inhibits the interaction of a PD-1 axis binding partner with either one or more of its binding partner, so as to remove T-cell dysfunction resulting from signaling on the PD-1 signaling axis—with a result being to restore or enhance T-cell function (e.g., proliferation, cytokine production, target cell killing) As used herein, a PD-1 axis binding antagonist includes a PD-1 binding antagonist, a PD-L1 binding antagonist and a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. Specific examples of PD-1 binding antagonists are provided infra.

The term “PD-L1 binding antagonist” refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. Specific examples of PD-L1 binding antagonists are provided infra.

The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “effective amount” is at least the minimum amount required to effect a measurable improvement or prevention of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “in conjunction with” or “in combination with” refers to administration of one treatment modality in addition to another treatment modality. As such, “in conjunction with” or “in combination with” refers to administration of one treatment modality before, during, or after administration of the other treatment modality to the individual.

A “disorder” is any condition that would benefit from treatment including, but not limited to, chronic and acute disorders or diseases including those pathological conditions which predispose the mammal to the disorder in question.

The terms “cell proliferative disorder” and “proliferative disorder” refer to disorders that are associated with some degree of abnormal cell proliferation. In one embodiment, the cell proliferative disorder is cancer. In one embodiment, the cell proliferative disorder is a tumor.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

A “subject” or an “individual” for purposes of treatment refers to any animal classified as a mammal, including humans, domestic and farm animals, and zoo, sports, or pet animals, such as dogs, horses, cats, cows, etc. Preferably, the mammal is human.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any mammalian species can be assigned to one of two clearly distinct types, called kappa (“κ”) and lambda (“λ”), based on the amino acid sequences of their constant domains.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen binding region thereof. In some embodiments, the antibody fragment described herein is an antigen-binding fragment. Examples of antibody fragments include Fab, Fab′, F(ab′)2, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthün, in The Pharmacology of Monoclonal Antibodies, vol. 113, Rosenburg and Moore eds., (Springer-Verlag, New York, 1994), pp. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “species-dependent antibody” is one which has a stronger binding affinity for an antigen from a first mammalian species than it has for a homologue of that antigen from a second mammalian species. Normally, the species-dependent antibody “binds specifically” to a human antigen (e.g., has a binding affinity (Kd) value of no more than about 1×10-7 M, preferably no more than about 1×10-8 M and preferably no more than about 1×10-9 M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above, but preferably is a humanized or human antibody.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat Numbering) |
| H1 | H31-H35 | H26-H35 | H26-H32 | H30-H35 |
| | | | | (Chothia Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

The expression “linear antibodies” refers to the antibodies described in Zapata et al. (1995 Protein Eng, 8(10):1057-1062). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some embodiments, the sample is a sample obtained from the cancer of an individual (e.g., a tumor sample) that comprises tumor cells and, optionally, tumor-infiltrating immune cells. For example, the sample can be a tumor specimen that is embedded in a paraffin block, or that includes freshly cut, serial unstained sections. In some embodiments, the sample is from a biopsy and includes 50 or more viable tumor cells (e.g., from a core-needle biopsy and optionally embedded in a paraffin block; excisional, incisional, punch, or forceps biopsy; or a tumor tissue resection).

By “tissue sample”, “tissue specimen” or “cell sample” is meant a collection of similar cells obtained from a tissue, for example a tumor, of a subject or individual. The source of the tissue or cell sample may be solid tissue (e.g., a tumor) as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A patient who “does not have an effective response” to treatment refers to a patient who does not have any one of extending survival (including overall survival and progression free survival); resulting in an objective response (including a complete response or a partial response); or improving signs or symptoms of cancer.

A “functional Fc region” possesses an “effector function” of a native sequence Fc region. Exemplary “effector functions” include C1q binding; CDC; Fc receptor binding; ADCC; phagocytosis; down regulation of cell surface receptors (e.g. B cell receptor; BCR), etc. Such effector functions generally require the Fc region to be combined with a binding domain (e.g., an antibody variable domain) and can be assessed using various assays as disclosed, for example, in definitions herein.

A cancer or biological sample which “has human effector cells” is one which, in a diagnostic test, has human effector cells present in the sample (e.g., infiltrating human effector cells).

A cancer or biological sample which “has FcR-expressing cells” is one which, in a diagnostic test, has FcR-expressing present in the sample (e.g., infiltrating FcR-expressing cells). In some embodiments, FcR is FcγR. In some embodiments, FcR is an activating FcγR.

### II. Methods of Inducing Neoepitope-Specific Immune Responses

Provided herein is a method for inducing neoepitope-specific CD8+ T cells in an individual with a tumor. In certain embodiments, the method includes the step of administering to the individual an effective amount of an RNA vaccine, wherein the vaccine includes one or more polynucleotides encoding one or more neoepitopes resulting from cancer-specific somatic mutations present in a tumor specimen obtained from the individual. In certain embodiments, at least about 1% (e.g., any of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, or more) of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine. In certain embodiments, about 1% to about 6% (e.g., any of about 1%, about 2%, about 3%, about 4%, about 5%, or about 6%) of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine. In some embodiments, the peripheral blood sample obtained from the individual after administration of the RNA vaccine contains about 5% or about 6% CD8+ T cells that are specific for at least one of the neoepitopes encoded by the one or more polynucleotides of the RNA vaccine.

In certain embodiments, at least about 0.1% (e.g., any of at least about 0.1%, at least about 0.18%, at least about 0.2%, at least about 0.27%, at least about 0.29%, at least about 0.3%, at least about 0.4%, at least about 0.5%, at least about 0.6%, at least about 0.7%, at least about 0.8%, at least about 0.87%, at least about 0.9%, at least about 1%, at least about 1.25%, at least about 1.5%, at least about 1.75%, at least about 2%, at least about 2.25%, at least about 2.5%, at least about 2.5%, at least about 2.75%, at least about 3%, at least about 3.25%, at least about 3.5%, at least about 3.75%, at least about 4%, at least about 4.25%, at least about 4.5%, at least about 4.75%, at least about 5%, at least about 5.25%, at least about 5.5%, at least about 5.67%, or more) of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine.

In certain embodiments, at least about 0.27% (e.g., any of at least about 0.27%, at least about 0.29%, at least about 0.3%, at least about 0.4%, at least about 0.5%, at least about 0.6%, at least about 0.7%, at least about 0.8%, at least about 0.87%, at least about 0.9%, at least about 1%, at least about 1.25%, at least about 1.5%, at least about 1.75%, at least about 2%, at least about 2.25%, at least about 2.5%, at least about 2.5%, at least about 2.75%, at least about 3%, at least about 3.25%, at least about 3.5%, at least about 3.75%, at least about 4%, at least about 4.25%, at least about 4.5%, at least about 4.75%, at least about 5%, at least about 5.25%, at least about 5.5%, at least about 5.67%, or more) of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine.

In certain embodiments, between about 0.1% to about 5.67% (e.g., any of about 0.1%, about 0.18%, about 0.2%, about 0.27%, about 0.29%, about 0.3%, about 0.4%, about 0.5%, about 0.6%, about 0.7%, about 0.8%, about 0.87%, about 0.9%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, or about 5.67%) of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine.

In certain embodiments, between about 0.27% to about 5.67% (e.g., any of about 0.27%, about 0.29%, about 0.3%, about 0.4%, about 0.5%, about 0.6%, about 0.7%, about 0.8%, about 0.87%, about 0.9%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, or about 5.67%) of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine.

In certain embodiments, about 0.18% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine. In certain embodiments, about 0.27% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine. In certain embodiments, about 0.29% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine. In certain embodiments, about 0.87% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine. In certain embodiments, about 1.89% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine. In certain embodiments, about 3.1% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine. In certain embodiments, about 5.67% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine. In certain embodiments, about 1.95% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine. In certain embodiments, about 2.49% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine. In certain embodiments, about 4.7% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine. In certain embodiments, about 2.2% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine.

The neoepitope-specific CD8+ T cells may be detected in the peripheral blood sample obtained from the individual after administration of the RNA vaccine by any method known in the art, such as ex vivo ELISPOT or MHC multimer analysis. In some embodiments, the neoepitope-specific CD8+ T cells in the peripheral blood sample obtained from the individual after administration of the RNA vaccine are specific for any of 1, 2, 3, 4, 5, 6, 7, 8, 9, 10, 11, 12, 13, 14, 15, 16, 17, 18, 19, or 20 of the neoepitopes encoded by the one or more polynucleotides of the RNA vaccine. In some embodiments, the neoepitope-specific CD8+ T cells in the peripheral blood sample obtained from the individual after administration of the RNA vaccine are specific for any of between 1 to 9, 1 to 8, 1 to 7, 1 to 6, 1 to 5, 1 to 4, 1 to 3, or 1 to 2 of the neoepitopes encoded by the one or more polynucleotides of the RNA vaccine. In some embodiments, the neoepitope-specific CD8+ T cells in the peripheral blood sample obtained from the individual after administration of the RNA vaccine are specific for about 2.6 or about 3 of the neoepitopes encoded by the one or more polynucleotides of the RNA vaccine.

In some embodiments, the neoepitope-specific CD8+ T cells in the peripheral blood sample obtained from the individual after administration of the RNA vaccine are specific for any of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or more of the neoepitopes encoded by the one or more polynucleotides of the RNA vaccine. In some embodiments, the neoepitope-specific CD8+ T cells in the peripheral blood sample obtained from the individual after administration of the RNA vaccine are specific for any of between about 5% to about 70% (e.g., any of about 5%, about 10%, about 15%, about 20%, about 25%, about 30%, about 35%, about 40%, about 45%, about 50%, about 55%, about 60%, about 65%, or about 70%) of the neoepitopes encoded by the one or more polynucleotides of the RNA vaccine. In some embodiments, the neoepitope-specific CD8+ T cells in the peripheral blood sample obtained from the individual after administration of the RNA vaccine are specific for any of between about 5% to about 35% (e.g., any of about 5%, about 10%, about 15%, about 20%, about 25%, about 30%, or about 35%) of the neoepitopes encoded by the one or more polynucleotides of the RNA vaccine.

In some embodiments, administration of the RNA vaccine to an individual according to the methods provided herein results in an induction (e.g., an increase) of neoepitope-specific CD4+ T cells that are specific for at least one of the neoepitopes encoded by the one or more polynucleotides of the RNA vaccine compared to prior to administration of the RNA vaccine. In some embodiments, the neoepitope-specific CD4+ T cells are detected in the peripheral blood of the individual. In some embodiments, the neoepitope-specific CD4+ T cells are detected in a peripheral blood sample obtained from the individual. In some embodiments, the neoepitope-specific CD4+ T cells in the peripheral blood sample obtained from the individual after administration of the RNA vaccine are specific for any of 1, 2, 3, 4, 5, 6, 7, 8, 9, 10, 11, 12, 13, 14, 15, 16, 17, 18, 19, or 20 of the neoepitopes encoded by the one or more polynucleotides of the RNA vaccine. In some embodiments, the neoepitope-specific CD4+ T cells in the peripheral blood sample obtained from the individual are detected by ex vivo ELISPOT analysis. In some embodiments, administration of the RNA vaccine to an individual according to the methods provided herein results in an induction (e.g., an increase) of neoepitope-specific CD4+ T cells that are specific for at least one of the neoepitopes encoded by the one or more polynucleotides of the RNA vaccine of any of at least about 1.1-fold, at least about 1.2-fold, at least about 1.3-fold, at least about 1.4-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, or more, compared to prior to administration of the RNA vaccine. In some embodiments, administration of the RNA vaccine to an individual according to the methods provided herein results in an induction (e.g., an increase) of neoepitope-specific CD4+ T cells that are specific for at least one of the neoepitopes encoded by the one or more polynucleotides of the RNA vaccine of any of at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 110-fold, at least about 120-fold, at least about 130-fold, at least about 140-fold, at least about 150-fold, at least about 160-fold, at least about 170-fold, at least about 180-fold, at least about 190-fold, at least about 200-fold, at least about 210-fold, at least about 220-fold, at least about 230-fold, at least about 240-fold, at least about 250-fold, at least about 260-fold, at least about 270-fold, at least about 280-fold, at least about 290-fold, at least about 300-fold, or more, compared to prior to administration of the RNA vaccine. In some embodiments, administration of the RNA vaccine to an individual according to the methods provided herein results in an induction (e.g., an increase) of neoepitope-specific CD4+ T cells that are specific for at least one of the neoepitopes encoded by the one or more polynucleotides of the RNA vaccine of any of at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, or more, compared to prior to administration of the RNA vaccine.

In some embodiments, administration of the RNA vaccine to a plurality of individuals according to the methods provided herein results in induction (e.g., an increase) of neoepitope-specific CD4+ and/or CD8+ T cells in the peripheral blood of at least about 70% of the individuals in the plurality, e.g., any of at least about 70%, at least about 75%, at least about 80%, at least about 85%, at least about 90%, at least about 95%, at least about 99%, or 100% of the individuals in the plurality. In some embodiments, administration of the RNA vaccine to a plurality of individuals according to the methods provided herein results in induction (e.g., an increase) of neoepitope-specific CD4+ and/or CD8+ T cells in the peripheral blood of at least about 73% of the individuals in the plurality. In some embodiments, administration of the RNA vaccine to a plurality of individuals according to the methods provided herein results in induction (e.g., an increase) of neoepitope-specific CD4+ and/or CD8+ T cells in the peripheral blood of at least about 86% of the individuals in the plurality. In some embodiments, the induction of neoepitope-specific CD4+ and/or CD8+ T cells in peripheral blood is assessed by ex vivo ELISPOT or MHC multimer analysis. In some embodiments, the induction (e.g., increase) of neoepitope-specific CD4+ and/or CD8+ T cells in peripheral blood comprises an increase of neoepitope-specific CD4+ and/or CD8+ T cells in the peripheral blood of an individual after administration of the RNA vaccine of any of at least about 1.1-fold, at least about 1.2-fold, at least about 1.3-fold, at least about 1.4-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 110-fold, at least about 120-fold, at least about 130-fold, at least about 140-fold, at least about 150-fold, at least about 160-fold, at least about 170-fold, at least about 180-fold, at least about 190-fold, at least about 200-fold, at least about 210-fold, at least about 220-fold, at least about 230-fold, at least about 240-fold, at least about 250-fold, at least about 260-fold, at least about 270-fold, at least about 280-fold, at least about 290-fold, at least about 300-fold, or more, compared to prior to administration of the RNA vaccine. In some embodiments, the induction (e.g., increase) of neoepitope-specific CD4+ and/or CD8+ T cells in peripheral blood comprises an increase of neoepitope-specific CD4+ and/or CD8+ T cells in the peripheral blood of an individual after administration of the RNA vaccine of any of at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, or more, compared to prior to administration of the RNA vaccine.

In some embodiments, administration of the RNA vaccine according to the methods provided herein results in an increase in the levels of one or more inflammatory cytokines. Examples of inflammatory cytokines include, without limitation, IFNγ (i.e., IFNg), IFNα (i.e., IFNα), IL-12, or IL-6. In some embodiments, administration of the RNA vaccine to an individual according to the methods provided herein results in an increase in the level of one or more inflammatory cytokines (e.g., IFNγ, IFNα, IL-12, and/or IL-6) in the peripheral blood (e.g., in plasma) of the individual compared to the level of the one or more inflammatory cytokines prior to administration of the RNA vaccine. In some embodiments, the increase in the level of the one or more inflammatory cytokines (e.g., IFNγ, IFNα, IL-12, and/or IL-6) after administration of a dose of the RNA vaccine is an increase of any of at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, or more, compared to the level of the one or more inflammatory cytokines (e.g., IFNγ, IFNα, IL-12, and/or IL-6) before administration of a dose of the RNA vaccine. In some embodiments, the increase in the level of the one or more inflammatory cytokines (e.g., IFNγ, IFNα, IL-12, and/or IL-6) after administration of a dose of the RNA vaccine is an increase of any of at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 110-fold, at least about 120-fold, at least about 130-fold, at least about 140-fold, at least about 150-fold, at least about 160-fold, at least about 170-fold, at least about 180-fold, at least about 190-fold, at least about 200-fold, at least about 210-fold, at least about 220-fold, at least about 230-fold, at least about 240-fold, at least about 250-fold, at least about 260-fold, at least about 270-fold, at least about 280-fold, at least about 290-fold, at least about 300-fold, or more, compared to the level of the one or more inflammatory cytokines (e.g., IFNγ, IFNα, IL-12, and/or IL-6) before administration of a dose of the RNA vaccine. In some embodiments, the increase in the level of the one or more inflammatory cytokines (e.g., IFNγ, IFNα, IL-12, and/or IL-6) is present in the peripheral blood (e.g., in plasma) of the individual at any of about 4 hours, about 5 hours, about 6 hours, or more after administration of a dose of the RNA vaccine. The levels of inflammatory cytokines (e.g., IFNγ, IFNα, IL-12, and/or IL-6) in peripheral blood (e.g., in plasma) may be quantified using any suitable method known in the art, including immunoassays such as ELISA, aptamer-based assays, Western blotting, and mass spectrometry. In some embodiments, the levels of inflammatory cytokines (e.g., IFNγ, IFNα, IL-12, and/or IL-6) in peripheral blood (e.g., in plasma) are quantified using ELISA assays.

Also provided herein is a method for inducing trafficking of neoepitope-specific CD8+ T cells to a tumor in an individual. In certain embodiments, the method includes the step of administering to the individual an effective amount of an RNA vaccine, wherein the RNA vaccine includes one or more polynucleotides encoding one or more neoepitopes resulting from cancer-specific somatic mutations present in a tumor specimen obtained from the individual. In certain embodiments, the neoepitope-specific CD8+ T cells trafficked to the tumor after administration of the RNA vaccine are specific for at least one of the neoepitopes encoded by the one or more polynucleotides of the RNA vaccine. In certain embodiments, the neoepitope-specific CD8+ T cells trafficked to the tumor after administration of the RNA vaccine are specific for any of 1, 2, 3, 4, 5, 6, 7, 8, 9, 10, 11, 12, 13, 14, 15, 16, 17, 18, 19, or 20 of the neoepitopes encoded by the one or more polynucleotides of the RNA vaccine. Trafficking of neoepitope-specific CD8+ T cells to a tumor in an individual may be measured by any method known in the art, e.g., as described in Cowell L G (2019) Cancer Res, 1457.2019. For example, T-cell receptors in a sample taken from the tumor can be sequenced to identify and measure the frequency of T-cell receptors that are specific for at least one of the neoepitopes encoded by the one or more polynucleotides of the RNA vaccine.

In some embodiments of the methods provided herein, the neoepitope-specific CD8+ T cells have a memory phenotype (e.g., the neoepitope-specific T cells are CD8+ memory T cells). In certain embodiments, the neoepitope-specific CD8+ T cells having a memory phenotype are CD45RO positive and CCR7 negative. In certain embodiments, the neoepitope-specific CD8+ T cells having a memory phenotype are effector memory T cells (i.e., Tₑₘ). In certain embodiments, the memory phenotype of neoepitope-specific CD8+ T cells may be determined using any markers known in the art. The memory phenotype (e.g., CD45RO positive and CCR7 negative) may be determined using any method known in the art, such as immunohistochemistry, fluorescence-activated cell sorting, and flow cytometry.

In some embodiments of the methods provided herein, the individual has a tumor with a low to intermediate mutational burden. In certain embodiments, the mutational burden of a tumor is determined by quantifying somatic mutations in the tumor. In certain embodiments, the individual has a tumor with 300 somatic mutations or less (e.g., any of 300 or less, 250 or less, 200 or less, 150 or less, 100 or less, 50 or less, 25 or less, 10 or less, 5 or less, or 1 somatic mutation). In certain embodiments, the individual has a tumor with at least about 100 (e.g., any of at least about 100, at least about 200, at least about 300, at least about 400, at least about 500, at least about 600, at least about 700, at least about 800, at least about 900, at least about 1000, or more) somatic mutations. In certain embodiments, the individual has a tumor with up to 1000 somatic mutations (e.g., any of 1 or more, 10 or more, 20 or more, 40 or more, 50 or more, 100 or more, 150 or more, 200 or more, 300 or more, 400 or more, 500 or more, 600 or more, 700 or more, 800 or more, 900 or more, or 1000 somatic mutations). In certain embodiments, the individual has a tumor with between about 100 and about 2000 (e.g., any of about 100, about 200, about 300, about 400, about 500, about 600, about 700, about 800, about 900, about 1000, about 1100, about 1200, about 1300, about 1400, about 1500, about 1600, about 1700, about 1800, about 1900, or about 2000) somatic mutations. In certain embodiments, the individual has a tumor with between about 300 and about 1000 somatic mutations. The mutational burden of a tumor may be determined using any method known in the art, such as whole exome sequencing (WES).

In some embodiments of the methods provided herein, the individual has a low tumor burden. In certain embodiments, the individual has a tumor burden that is 25% or less (e.g., any of 25% or less, 20% or less, 15% or less, 10% or less, 5% or less, 2.5% or less, or 1% or less) of the median tumor burden for individuals having the same type of tumor or cancer as the tumor in the individual. In certain embodiments, the individual has a tumor burden that is 50% or less (e.g., any of 50% or less, 45% or less, 40% or less, 35% or less, 30% or less, 25% or less, 20% or less, 15% or less, 10% or less, 5% or less, 2.5% or less, or 1% or less) than the median tumor burden for individuals having the same type of tumor or cancer as the tumor in the individual. Tumor burden in the individual may be measured using any method known in the art, e.g., as described in Cai et al., (2018) Chronic Diseases and Translational Medicine, 4(1):18-28; Nishino M (2018) ASCO Educational Book, 28:1019-29; and Akbar et al., (2019) Scientific Reports, 9:14099. For example, tumor burden may be measured by quantifying the tumor diameter (e.g., the largest tumor diameter, and/or the combined tumor diameter), quantifying the tumor volume, and quantifying the number of metastases. In certain embodiments, tumor burden in the individual is measured manually (e.g., by a clinician and/or a radiologist) or automatically (e.g., using a computational approach). As used herein, tumor burden in the individual also refers to tumor load in the individual.

In some embodiments of the methods provided herein, the tumor has low or negative PD-L1 expression. In certain embodiments, less than about 5% (e.g., any of less than about 5%, less than about 4.5%, less than about 4%, less than about 3.5%, less than about 3%, less than about 2.5%, less than about 2%, less than about 1.5%, less than about 1%, less than about 0.5%, or less than about 0.25%) of tumor cells in a sample obtained from the tumor express PD-L1. In certain embodiments, less than about 5% (e.g., any of less than about 5%, less than about 4.5%, less than about 4%, less than about 3.5%, less than about 3%, less than about 2.5%, less than about 2%, less than about 1.5%, less than about 1%, less than about 0.5%, or less than about 0.25%) of immune cells in a sample obtained from the tumor express PD-L1. The percentage of tumor cells and/or immune cells in a sample obtained from the tumor that express PD-L1 may be determined according to any method known in the art, such as immunohistochemistry, fluorescence-activated cell sorting, or flow cytometry. In certain embodiments, the percentage of tumor cells or immune cells in a sample obtained from the tumor that express PD-L1 is determined using immunohistochemistry. In some embodiments, the percentage of tumor cells and/or immune cells in a sample obtained from the tumor that express PD-L1 may be determined by quantifying the level of membrane staining of PD-L1 by immunohistochemistry or any method known in the art. In some embodiments, the percentage of tumor cells and/or immune cells in a sample obtained from the tumor that express PD-L1 is determined using the Ventana SP142 assay.

Administration of RNA Vaccine & PD-1 Axis Antagonist

In some embodiments of the methods provided herein, the RNA vaccine is administered to the individual at a dose of between about 15 μg to about 100 μg (e.g., any of about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, or about 100 μg). In some embodiments, the RNA vaccine is administered to the individual at a dose of about 15 μg, about 25 μg, about 38 μg, about 50 μg, about 75 μg, or about 100 μg. In certain embodiments, the RNA vaccine is administered intravenously to the individual.

In some embodiments of the methods provided herein, the RNA vaccine is administered to the individual at an interval of 7 days or 1 week. In certain embodiments, the RNA vaccine is administered to the individual at an interval of 14 days or 2 weeks. In certain embodiments, the RNA vaccine is administered to the individual for 12 weeks or 84 days.

In some embodiments of the methods provided herein, the RNA vaccine is administered to the individual in four 21-day Cycles, wherein the RNA vaccine is administered to the individual on Days 1, 8, and 15 of Cycle 1; Days 1, 8, and 15 of Cycle 2; Days 1 and 15 of Cycle 3; and Day 1 of Cycle 4.

In some embodiments of the methods provided herein, the RNA vaccine is administered to the individual in 21-day Cycles, wherein the RNA vaccine is administered to the individual on Days 1, 8, and 15 of Cycle 1; Days 1, 8, and 15 of Cycle 2; Days 1 and 15 of Cycle 3; and Day 1 of Cycle 7. In some embodiments, the methods provided herein further include administering the RNA vaccine on Day 1 of Cycle 13, and every 24 weeks or 168 days thereafter. In some embodiments, administration of the RNA vaccine continues until an occurrence of disease progression in the individual.

In some embodiments of the methods provided herein, the RNA vaccine is administered to the individual in 21-day Cycles, wherein the RNA vaccine is administered to the individual on Days 1, 8, and 15 of Cycle 2; Days 1 and 15 of Cycle 3; and Day 1 of Cycle 7. In some embodiments, the methods provided herein further include administering the RNA vaccine on Day 1 of Cycle 13, and every 24 weeks or 168 days thereafter. In some embodiments, administration of the RNA vaccine continues until an occurrence of disease progression in the individual.

In some embodiments of the methods provided herein, the RNA vaccine is administered to the individual in an induction stage and a maintenance stage after the induction stage, wherein the RNA vaccine is administered to the individual during the induction stage at an interval of 1 or 2 weeks, and wherein the RNA vaccine is administered to the individual during the maintenance stage at an interval of 24 weeks. In certain embodiments, the RNA vaccine is administered to the individual in an induction stage and a maintenance stage after the induction stage, wherein the RNA vaccine is administered to the individual during the induction stage at an interval of 7 days or 14 days, and wherein the RNA vaccine is administered to the individual during the maintenance stage at an interval of 168 days.

In some embodiments of the methods provided herein, the RNA vaccine is administered to the individual in an induction stage and a maintenance stage after the induction stage, wherein the RNA vaccine is administered to the individual during the induction stage in four 21-day Cycles, wherein the RNA vaccine is administered to the individual during the induction stage on Days 1, 8, and 15 of Cycle 1; Days 1, 8, and 15 of Cycle 2; Days 1 and 15 of Cycle 3; and Day 1 of Cycle 4; and wherein the RNA vaccine is administered to the individual during the maintenance stage on Day 1 of Cycle 5 and once every 24 weeks or 168 days thereafter.

In some embodiments of the methods provided herein, the RNA vaccine is administered to the individual in an induction stage and a maintenance stage after the induction stage, wherein the RNA vaccine is administered to the individual in 21-day Cycles; wherein, during the induction stage, the RNA vaccine is administered to the individual on Days 1, 8, and 15 of Cycle 1; Days 1, 8, and 15 of Cycle 2; Days 1 and 15 of Cycle 3; and Day 1 of Cycle 7; and wherein, during the maintenance stage, the RNA vaccine is administered to the individual on Day 1 of Cycle 13 and once every 24 weeks or 168 days thereafter. In some embodiments, the induction stage includes up to 9 doses of the RNA vaccine. In some embodiments, the maintenance stage continues until an occurrence of disease progression in the individual.

In some embodiments of the methods provided herein, the RNA vaccine is administered to the individual in an induction stage and a maintenance stage after the induction stage, wherein the RNA vaccine is administered to the individual in 21-day Cycles; wherein, during the induction stage, the RNA vaccine is administered to the individual on Days 1, 8, and 15 of Cycle 2; Days 1 and 15 of Cycle 3; and Day 1 of Cycle 7; and wherein, during the maintenance stage, the RNA vaccine is administered to the individual on Day 1 of Cycle 13 and once every 24 weeks or 168 days thereafter. In some embodiments, the induction stage includes up to 9 doses of the RNA vaccine. In some embodiments, the maintenance stage continues until an occurrence of disease progression in the individual.

In certain embodiments, the maintenance stage is continued until disease progression or withdrawal from treatment by the individual.

In certain embodiments, the individual is administered at least 3 doses of the RNA vaccine. In certain embodiments, the individual is administered at least 6 doses of the RNA vaccine. In certain embodiments, the individual is administered at least 9 doses of the RNA vaccine. In certain embodiments, the individual is administered about 3 doses of the RNA vaccine. In certain embodiments, the individual is administered about 6 doses of the RNA vaccine. In certain embodiments, the individual is administered about 9 doses of the RNA vaccine. In certain embodiments, the induction stage includes up to 9 doses of the RNA vaccine. In certain embodiments, the individual is administered less than 9 doses of the RNA vaccine.

In some embodiments of the methods provided herein, the methods further comprise a step of administering a PD-1 axis binding antagonist to the individual. In certain embodiments, the PD-1 axis binding antagonist is administered intravenously to the individual.

In certain embodiments, the PD-1 axis binding antagonist is a PD-1 binding antagonist. In certain embodiments, the PD-1 binding antagonist is an anti-PD-1 antibody. In certain embodiments, the anti-PD-1 antibody is nivolumab or pembrolizumab.

In certain embodiments of the methods provided herein, the PD-1 axis binding antagonist is a PD-L1 binding antagonist. In certain embodiments, the PD-L1 binding antagonist is an anti-PD-L1 antibody. In certain embodiments, the anti-PD-L1 antibody is avelumab or durvalumab. In certain embodiments, the anti-PD-L1 antibody includes: (a) a heavy chain variable region (VH) that includes an HVR-H1 including an amino acid sequence of GFTFSDSWIH (SEQ ID NO:1), an HVR-2 including an amino acid sequence of AWISPYGGSTYYADSVKG (SEQ ID NO:2), and HVR-3 including an amino acid RHWPGGFDY (SEQ ID NO:3), and (b) a light chain variable region (VL) that includes an HVR-L1 including an amino acid sequence of RASQDVSTAVA (SEQ ID NO:4), an HVR-L2 including an amino acid sequence of SASFLYS (SEQ ID NO:5), and an HVR-L3 including an amino acid sequence of QQYLYHPAT (SEQ ID NO:6). In certain embodiments, the anti-PD-L1 antibody includes a heavy chain variable region (V_{H}) including an amino acid sequence of SEQ ID NO:7 and a light chain variable region (VL) including an amino acid sequence of SEQ ID NO:8. In certain embodiments, the anti-PD-L1 antibody is atezolizumab. In certain embodiments, the anti-PD-L1 antibody is administered to the individual at a dose of about 1200 mg.

In certain embodiments, the PD-1 axis binding antagonist is administered to the individual at an interval of 21 days or 3 weeks (e.g., on Day 1 of each 21-day cycle).

In some embodiments of the methods provided herein, the PD-1 axis binding antagonist is atezolizumab, and the atezolizumab is administered to the individual in 21-day cycles, wherein atezolizumab is administered on Day 1 of each of Cycles 1, 2, 3, 4, 5, 6, 7, 8, 9, 10, 11, and 12. In some embodiments, the atezolizumab is further administered on Day 1 of Cycle 13, and every 3 weeks or 21 days thereafter. In some embodiments, administration of atezolizumab continues until an occurrence of disease progression in the individual.

In some embodiments of the methods provided herein, the PD-1 axis binding antagonist is atezolizumab, and the atezolizumab is administered to the individual in 21-day cycles during an induction stage and during a maintenance stage after the induction stage; wherein, during the induction stage, atezolizumab is administered on Day 1 of each of Cycles 1, 2, 3, 4, 5, 6, 7, 8, 9, 10, 11, and 12; and wherein, during the maintenance stage after the induction stage, atezolizumab is administered on Day 1 of Cycle 13, and every 3 weeks or 21 days thereafter. In some embodiments, the maintenance stage continues until an occurrence of disease progression in the individual.

In some embodiments, disease progression is assessed according to the Response Evaluation Criteria for Solid Tumors Version 1.1 (RECIST v1.1).

Response to Administration

In some embodiments of the methods for inducing neoepitope-specific CD8+ T cells in an individual with a tumor, methods for inducing trafficking of neoepitope-specific CD8+ T cells to a tumor in an individual, and/or methods of treatment (see, e.g., Section VII, below) provided herein, administration of the RNA vaccine results in a complete response (CR) or partial response (PR) in the individual. In certain embodiments, administration of the RNA vaccine results in a complete response (CR) in the individual. In some embodiments, administration of the RNA vaccine results in a partial response (PR) in the individual. In certain embodiments, complete or partial responses are assessed according to the Response Evaluation Criteria for Solid Tumors Version 1.1 (RECIST v1.1) or the immune-modified RECIST. In certain embodiments, complete or partial responses are assessed from baseline until the last dose of RNA vaccine, initiation of another systemic anti-cancer therapy, disease progression, or death.

In some embodiments of the methods provided herein, administration of the RNA vaccine to a plurality of individuals with a tumor results in at least about 4% (e.g., any of at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or more) of individuals within the plurality having a complete response or a partial response.

In certain embodiments, a complete response or a partial response persists for about 6 months or more (e.g., any of about 6 months or more, about 7 months or more, about 8 months or more, about 9 months or more, about 10 months or more, about 11 months or more, about 12 months or more, about 14 months or more, about 15 months or more, about 20 months or more, about 24 months or more, about 30 months or more, about 36 months or more, about 42 months or more, about 48 months or more, about 54 months or more, or about 60 months or more). In certain embodiments, a complete response persists for about 10 months or more.

In some embodiments, administration of the RNA vaccine to a plurality of individuals with a tumor results in at least about 20% (e.g., any of at least about 20%, at least about 30%, at least about 40%, at least about 50%, at least about 60%, at least about 70%, at least about 80%, at least about 90%, at least about 95%, at least about 99%, or 100%) of individuals within the plurality having stable disease. In certain embodiments, administration of the RNA vaccine to a plurality of individuals with a tumor results in at least about 42% of individuals within the plurality having stable disease. In certain embodiments, administration of the RNA vaccine to a plurality of individuals with a tumor results in at least about 49% of individuals within the plurality having stable disease.

In some embodiments, administration of the RNA vaccine to a plurality of individuals with a tumor results in at least 60% (e.g., any of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) of individuals having an RNA-vaccine-induced neoantigen-specific CD8+ T cell response (e.g., wherein a peripheral blood sample obtained from the individual after administration of the RNA vaccine contains at least about 1% (e.g., any of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, or more) CD8+ T cells that are specific for at least one of the neoepitopes encoded by the one or more polynucleotides of the RNA vaccine; or wherein neoepitope-specific CD8+ T cells trafficked to the tumor after administration of the RNA vaccine are specific for at least one of the neoepitopes encoded by the one or more polynucleotides of the RNA vaccine). In some embodiments, administration of the RNA vaccine to a plurality of individuals with a tumor results in at least 60% (e.g., any of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) of individuals having an RNA-vaccine-induced neoantigen-specific CD8+ T cell response (e.g., wherein a peripheral blood sample obtained from the individual after administration of the RNA vaccine contains about 1% to about 6% CD8+ T cells that are specific for at least one of the neoepitopes encoded by the one or more polynucleotides of the RNA vaccine; or wherein neoepitope-specific CD8+ T cells trafficked to the tumor after administration of the RNA vaccine are specific for at least one of the neoepitopes encoded by the one or more polynucleotides of the RNA vaccine). In some embodiments, administration of the RNA vaccine to a plurality of individuals with a tumor results in about 77% of individuals having an RNA-vaccine-induced neoantigen-specific CD8+ T cell response. In some embodiments, administration of the RNA vaccine to a plurality of individuals with a tumor results in about 87% of individuals having an RNA-vaccine-induced neoantigen-specific CD8+ T cell response. The RNA-vaccine-induced neoantigen-specific CD8+ T cell response may be assayed using any method known in the art, for example using an ELISPOT assay, T cell receptor sequencing, or MHC multimer analysis.

In some embodiments, administration of the RNA vaccine to a plurality of individuals according to the methods provided herein results in induction of neoepitope-specific CD4+ and/or CD8+ T cells in the peripheral blood of at least about 70% of the individuals in the plurality, e.g., any of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the individuals in the plurality. In some embodiments, administration of the RNA vaccine to a plurality of individuals according to the methods provided herein results in induction of neoepitope-specific CD4+ and/or CD8+ T cells in the peripheral blood of at least about 73% of the individuals in the plurality. In some embodiments, administration of the RNA vaccine to a plurality of individuals according to the methods provided herein results in induction of neoepitope-specific CD4+ and/or CD8+ T cells in the peripheral blood of at least about 86% of the individuals in the plurality. The RNA-vaccine-induced neoantigen-specific CD8+ and/or CD4+ T cell response may be assayed using any method known in the art, for example using an ELISPOT assay, T cell receptor sequencing, or MHC multimer analysis. In some embodiments, the induction of neoepitope-specific CD4+ and/or CD8+ T cells in peripheral blood is assessed by ex vivo ELISPOT or MHC multimer analysis.

In certain embodiments, administration of the RNA vaccine results in release of pro-inflammatory cytokines with each dose of RNA vaccine administered.

In some embodiments, administration of the RNA vaccine to a plurality of individuals with the tumor results in an increase in progression-free survival (PFS) (e.g., an increase in the mean or median PFS), compared to a plurality of individuals with the tumor not administered the RNA vaccine. In certain embodiments, PFS is measured in days, weeks, months, or years. In certain embodiments, PFS is determined according to RECIST v1.1. In certain embodiments, administration of the RNA vaccine to a plurality of individuals with the tumor results in an increase in the overall survival (e.g., an increase in the mean or median OS), compared to a plurality of individuals with the tumor not administered the RNA vaccine. In certain embodiments, overall survival is measured in days, weeks, months, or years. In certain embodiments, overall survival refers to the percentage of individuals that are alive at a specified time, e.g., days, weeks, months, or years after administration of the RNA vaccine.

In some embodiments, the treatment extends the progression free survival (PFS) and/or the overall survival (OS) of the individual, as compared to a treatment comprising administration of a PD-1 axis binding antagonist in the absence of an RNA vaccine. In some embodiments, the treatment improves overall response rate (ORR), as compared to a treatment comprising administration of a PD-1 axis binding antagonist in the absence of an RNA vaccine. In some embodiments, ORR refers to the proportion of patients with a complete response (CR) or partial response (PR). In some embodiments, the treatment extends the duration of response (DOR) in the individual, as compared to a treatment comprising administration of a PD-1 axis binding antagonist in the absence of an RNA vaccine. In some embodiments, the treatment improves health-related quality of life (HRQoL) score in the individual, as compared to a treatment comprising administration of a PD-1 axis binding antagonist in the absence of an RNA vaccine.

In some embodiments, administration of the RNA vaccine to a plurality of individuals according to the methods provided herein results in an objective response in at least about 2% of the individuals in the plurality (e.g., in any of at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the individuals in the plurality). In some embodiments, the tumor is a urothelial tumor (e.g., not previously treated with a checkpoint inhibitor), and administration of the RNA vaccine to a plurality of individuals results in an objective response in at least about 10% of the individuals in the plurality (e.g., in any of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the individuals in the plurality). In some embodiments, the tumor is a renal tumor (e.g., not previously treated with a checkpoint inhibitor), and administration of the RNA vaccine to a plurality of individuals results in an objective response in at least about 22% of the individuals in the plurality (e.g., in any of at least about 22%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the individuals in the plurality). In some embodiments, the tumor is a melanoma tumor (e.g., not previously treated with a checkpoint inhibitor), and administration of the RNA vaccine to a plurality of individuals results in an objective response in at least about 30% of the individuals in the plurality (e.g., in any of at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the individuals in the plurality). In some embodiments, the tumor is a TNBC tumor (e.g., not previously treated with a checkpoint inhibitor), and administration of the RNA vaccine to a plurality of individuals results in an objective response in at least about 4% of the individuals in the plurality (e.g., in any of at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the individuals in the plurality). In some embodiments, the tumor is an NSCLC tumor (e.g., not previously treated with a checkpoint inhibitor), and administration of the RNA vaccine to a plurality of individuals results in an objective response in at least about 10% of the individuals in the plurality (e.g., in any of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the individuals in the plurality). An objective response refers to an occurrence of a complete response or a partial response in the individual, according to the Response Evaluation Criteria in Solid Tumors (RECIST) v1.1 assessment criteria, see, e.g., Eisenhauer et al (2009) Eur J Cancer, 45:228-47.

Individuals with a Tumor

In certain embodiments of the methods provided herein, the individual is a human.

In some embodiments of the methods provided herein, the individual has a locally advanced, recurrent, or metastatic incurable malignancy. In some embodiments, the individual has a locally advanced or metastatic solid tumor or has one or more metastatic relapses. In certain embodiments, the tumor or the malignancy has progressed after at least one standard therapy prior to administration of the RNA vaccine. In certain embodiments standard therapy has proven ineffective, intolerable, or inappropriate for the individual prior to administration of the RNA vaccine. In certain embodiments, the individual has an Eastern Cooperative Oncology Group (ECOG) performance status of 0 or 1 prior to administration of the RNA vaccine. In certain embodiments, the individual has measurable disease according to RECIST v1.1 prior to administration of the RNA vaccine.

In some embodiments of the methods provided herein, the tumor is a non-small cell lung (NSCLC), bladder, renal, head and neck, sarcoma, breast, melanoma, prostate, ovarian, gastric, liver, or colorectal tumor. In some embodiments, the tumor is a breast tumor, and the breast tumor is a triple-negative breast (TNBC) tumor. In some embodiments of the methods provided herein, the tumor is a non-small cell lung (NSCLC), bladder, renal, head and neck, sarcoma, breast, melanoma, prostate, ovarian, gastric, liver, urothelial, colon, kidney, cervix, Merkel cell (MCC), endometrial, soft tissue sarcoma, esophageal, esophagogastric junction, bone sarcoma, thyroid, or colorectal tumor.

In some embodiments of the methods provided herein, prior to administration of the RNA vaccine, the individual has been treated with one or more cancer therapies. In some embodiments, prior to administration of the RNA vaccine, the individual has been treated with one or more cancer therapies or between 3 and 5 cancer therapies. In certain embodiments, prior to administration of the RNA vaccine, the individual has been treated with between about 1 to about 20 (e.g., about 1, about 2, about 3, about 4, about 5, about 6, about 7, about 8, about 9, about 10, about 11, about 12, about 13, about 14, about 15, about 16, about 17, about 18, about 19, about 20, or more) cancer therapies. In certain embodiments, prior to administration of the RNA vaccine, the individual has been treated with at least 1 cancer therapy. In certain embodiments, prior to administration of the RNA vaccine, the individual has been treated with about 3 cancer therapies. In certain embodiments, prior to administration of the RNA vaccine, the individual has been treated with about 5 cancer therapies. In some embodiments, prior to administration of the RNA vaccine, the individual has been treated with between 3 and 5 cancer therapies. In some embodiments, prior to administration of the RNA vaccine, the individual has been treated with between about 1 to about 17 (e.g., any of about 1, about 2, about 3, about 4, about 5, about 6, about 7, about 8, about 9, about 10, about 11, about 12, about 13, about 14, about 15, about 16, or about 17), or between about 1 to about 9 (e.g., any of about 1, about 2, about 3, about 4, about 5, about 6, about 7, about 8, or about 9) prior systemic cancer therapies. Examples of systemic cancer therapies include, without limitation, chemotherapy, hormone therapy, radiation therapy, targeted therapies, immunotherapies, or other treatments, e.g., as described in Palumbo et al (2013) Front Pharmacol, 4:57.

In some embodiments of the methods provided herein, prior to administration of the RNA vaccine, the individual has been treated with an immunotherapy. In some embodiments of the methods provided herein, prior to administration of the RNA vaccine, the individual has been treated with a checkpoint inhibitor therapy (e.g., an anti-PD-L1 therapy, an anti-PD-1 therapy, an anti-CTLA4 therapy, or any combination thereof). In certain embodiments, prior to administration of the RNA vaccine, the individual has not been treated with a checkpoint inhibitor therapy (e.g., an anti-PD-L1 therapy, an anti-PD-1 therapy, an anti-CTLA4 therapy, or any combination thereof).

In some embodiments of the methods provided herein, the tumor is a NSCLC tumor, and prior to administration of the RNA vaccine, the individual has not been treated with anti-PD-L1/PD-1 and/or anti-CTLA-4 therapies. In certain embodiments, the tumor is a NSCLC tumor, and prior to administration of the RNA vaccine, the individual has been treated with an anti-PD-L1/PD-1 therapy with or without an anti-CTLA-4 therapy.

In certain embodiments, the tumor is a TNBC tumor, and prior to administration of the RNA vaccine, the individual has not been previously treated with anti-PD-L1/PD-1 and/or anti-CTLA-4 therapies. In certain embodiments, the tumor is a TNBC tumor, and prior to administration of the RNA vaccine, the individual has been previously treated with an anti-PD-L1/PD-1 therapy with or without an anti-CTLA-4 therapy. As used herein, a TNBC tumor refers to an estrogen receptor (ER)-negative, progesterone receptor-negative, and human epidermal growth factor receptor 2 (HER2)-negative adenocarcinoma of the breast.

In certain embodiments, the tumor is a colorectal cancer tumor, and prior to administration of the RNA vaccine, the individual has not been previously treated with anti-PD-L1/PD-1 and/or anti-CTLA-4 therapies. In certain embodiments, the tumor is a colorectal cancer tumor, and prior to administration of the RNA vaccine, the individual has been previously treated with an anti-PD-L1/PD-1 therapy with or without an anti-CTLA-4 therapy.

In certain embodiments, the tumor is a head and neck squamous cell carcinoma, and prior to administration of the RNA vaccine, the individual has not been previously treated with anti-PDL1/PD-1 and/or anti-CTLA-4 therapies. In certain embodiments, the tumor is a head and neck squamous cell carcinoma and prior to administration of the RNA vaccine, the individual has been previously treated with an anti-PD-L1/PD-1 therapy with or without an anti-CTLA-4 therapy.

In certain embodiments, the tumor is an urothelial carcinoma tumor, and prior to administration of the RNA vaccine, the individual has not been previously treated with an anti-PD-L1/PD-1 therapy with or without an anti-CTLA-4 therapy. In certain embodiments, the tumor is an urothelial carcinoma tumor, and prior to administration of the RNA vaccine, the individual has been previously treated with an anti-PD-L1/PD-1 therapy with or without an anti-CTLA-4 therapy.

In certain embodiments, the tumor is a renal cell carcinoma, and prior to administration of the RNA vaccine, the individual has not been previously treated with anti-PD-L1/PD-1 and/or anti-CTLA-4 therapies. In certain embodiments, the tumor is a renal cell carcinoma, and prior to administration of the RNA vaccine, the individual has been previously treated with an anti-PD-L1/PD-1 therapy with or without an anti-CTLA-4 therapy.

In certain embodiments, the tumor is a melanoma tumor, and prior to administration of the RNA vaccine, the individual has not been previously treated with anti-PD-L1/PD-1 and/or anti-CTLA-4 therapies. In certain embodiments, the tumor is a melanoma tumor, and prior to administration of the RNA vaccine, the individual has been previously treated with anti-PD-L1/PD-1 and/or an anti-CTLA-4 therapies.

In certain embodiments, prior to administration of the RNA vaccine, the individual has been administered immunomodulators, such as toll-like receptor (TLR) agonists, inhibitors of indoleamine 2,3-dioxygenase (IDO)/tryptophan-2,3-dioxygenase (TDO), or agonists of OX40.

In some embodiments of the methods provided herein, the individual does not have clinically significant liver disease. In certain embodiments, the individual has not had a splenectomy prior to administration of the RNA vaccine. In certain embodiments, the individual does not have a primary immunodeficiency, either cellular (e.g., DiGeorge syndrome, T-negative severe combined immunodeficiency [SCID]) or a combined T- and B-cell immunodeficiency (e.g., T- and B-negative SCID, Wiskott Aldrich syndrome, ataxia telangiectasia, common variable immunodeficiency). In certain embodiments, the individual does not have a primary central nervous system (CNS) malignancy, untreated CNS metastases, or active CNS metastases. In certain embodiments, the individual does not have leptomeningeal disease. In certain embodiments, the individual does not have an autoimmune disease. In certain embodiments, the individual does not have idiopathic pulmonary fibrosis, pneumonitis, organizing pneumonia, or evidence of active pneumonitis on screening chest computed tomography (CT) scan; human immunodeficiency virus infection; active hepatitis B or C; active or latent tuberculosis infection; or a severe infection. In certain embodiments, the individual has not had an allogeneic bone marrow transplantation or a solid organ transplantation.

### III. RNA Vaccines

Certain aspects of the present disclosure relate to personalized cancer vaccines (PCVs). In some embodiments, the PCV is an RNA vaccine. Features of exemplary RNA vaccines are described infra. In some embodiments, the present disclosure provides an RNA polynucleotide comprising one or more of the features/sequences of the RNA vaccines described infra. In some embodiments, the RNA polynucleotide is a single-stranded mRNA polynucleotide. In other embodiments, the present disclosure provides a DNA polynucleotide encoding an RNA comprising one or more of the features/sequences of the RNA vaccines described infra.

Personalized cancer vaccines comprise individualized neoantigens (i.e., tumor-associated antigens (TAAs) that are specifically expressed in the patient's cancer) identified as having potential immunostimulatory activities. In the embodiments described herein, the PCV is a nucleic acid, e.g., messenger RNA. Accordingly, without wishing to be bound by theory, it is believed that upon administration, the personalized cancer vaccine (e.g., an RNA vaccine of the disclosure) is taken up and translated by antigen presenting cells (APCs) and the expressed protein is presented via major histocompatibility complex (MHC) molecules on the surface of the APCs. This leads to an induction of both cytotoxic T-lymphocyte (CTL)-and memory T-cell-dependent immune responses against cancer cells expressing the TAA(s).

PCVs (e.g., an RNA vaccine) typically include multiple neoantigen epitopes (“neoepitopes”), e.g., 2, 3, 4, 5, 6, 7, 8, 9, 10, 11, 12, 13, 14, 15, 16, 17, 18, 19, 20, 21, 22, 23, 24, 25, 26, 27, 28, 28, 29, or 30 neoepitopes or at least 2, 3, 4, 5, 6, 7, 8, 9, 10, 11, 12, 13, 14, 15, 16, 17, 18, 19, 20, 21, 22, 23, 24, 25, 26, 27, 28, 28, 29, or 30 neoepitopes, optionally with linker sequences between the individual neoepitopes. In some embodiments, a neoepitope as used herein refers to a novel epitope that is specific for a patient's cancer but not found in normal cells of the patient. In some embodiments, the neoepitope is presented to T cells when bound to MHC. In some embodiments, the PCV also includes a 5′ mRNA cap analogue, a 5′ UTR, a signal sequence, a domain to facilitate antigen expression, a 3′ UTR, and/or a polyA tail. In some embodiments, the RNA vaccine comprises one or more polynucleotides encoding 10-20 neoepitopes resulting from cancer-specific somatic mutations present in the tumor specimen. In some embodiments, the RNA vaccine comprises one or more polynucleotides encoding at least 5 neoepitopes resulting from cancer-specific somatic mutations present in the tumor specimen. In some embodiments, the RNA vaccine comprises one or more polynucleotides encoding 5-20 neoepitopes resulting from cancer-specific somatic mutations present in the tumor specimen. In some embodiments, the RNA vaccine comprises one or more polynucleotides encoding 5-10 neoepitopes resulting from cancer-specific somatic mutations present in the tumor specimen.

In some embodiments, the manufacture of an RNA vaccine of the present disclosure is a multi-step process, whereby somatic mutations in the patient's tumor are identified by next-generation sequencing (NGS) and immunogenic neoantigen epitopes (or “neoepitopes”) are predicted. The RNA cancer vaccine targeting the selected neoepitopes is manufactured on a per-patient basis. In some embodiments, the vaccine is an RNA-based cancer vaccine consisting of up to two messenger RNA molecules, each encoding up to 10 neoepitopes (for a total of up to 20 neoepitopes), which are specific to the patient's tumor.

In some embodiments, expressed non-synonymous mutations are identified by whole exome sequencing (WES) of tumor DNA and peripheral blood mononuclear cell (PBMC) DNA (as a source of healthy tissue from the patient) as well as tumor RNA sequencing (to assess expression). From the resulting list of mutant proteins, potential neoantigens are predicted using a bioinformatics workflow that ranks their likely immunogenicity on the basis of multiple factors, including the binding affinity of the predicted epitope to individual major histocompatibility complex (MHC) molecules, and the level of expression of the associated RNA. The mutation discovery, prioritization, and confirmation processes are complemented by a database that provides comprehensive information about expression levels of respective wild-type genes in healthy tissues. This information enables the development of a personalized risk mitigation strategy by removing target candidates with an unfavorable risk profile. Mutations occurring in proteins with a possible higher auto-immunity risk in critical organs are filtered out and not considered for vaccine production. In some embodiments, up to 20 MHCI and MHCII neoepitopes that are predicted to elicit CD8⁺ T-cell and/or CD4⁺ T-cell responses, respectively, for an individual patient are selected for inclusion into the vaccine. Vaccinating against multiple neoepitopes is expected to increase the breadth and magnitude of the overall immune response to PCV and may help to mitigate the risk of immune escape, which can occur when tumors are exposed to the selective pressure of an effective immune response (Tran E, Robbins P F, Lu Y C, et al. N Engl J Med 2016; 375:2255-62; Verdegaal E M, de Miranda N F, Visser M, et al. Nature 2016; 536:91-5).

In some embodiments, the RNA vaccine comprises one or more polynucleotide sequences encoding an amino acid linker. For example, amino acid linkers can be used between 2 tumor-specific neoepitope sequences, between a tumor-specific neoepitope sequence and a fusion protein tag (e.g., comprising sequence derived from an MHC complex polypeptide), or between a secretory signal peptide and a tumor-specific neoepitope sequence. In some embodiments, the RNA vaccine encodes multiple linkers. In some embodiments, the RNA vaccine comprises one or more polynucleotides encoding 5-20 neoepitopes resulting from cancer-specific somatic mutations present in the tumor specimen, and the polynucleotides encoding each epitope are separated by a polynucleotide encoding a linker sequence. In some embodiments, the RNA vaccine comprises one or more polynucleotides encoding 5-10 neoepitopes resulting from cancer-specific somatic mutations present in the tumor specimen, and the polynucleotides encoding each epitope are separated by a polynucleotide encoding a linker sequence. In some embodiments, polynucleotides encoding linker sequences are also present between the polynucleotides encoding an N-terminal fusion tag (e.g., a secretory signal peptide) and a polynucleotide encoding one of the neoepitopes and/or between a polynucleotide encoding one of the neoepitopes and the polynucleotides encoding a C-terminal fusion tag (e.g., comprising a portion of an MHC polypeptide). In some embodiments, two or more linkers encoded by the RNA vaccine comprise different sequences. In some embodiments, the RNA vaccine encodes multiple linkers, all of which share the same amino acid sequence.

A variety of linker sequences are known in the art. In some embodiments, the linker is a flexible linker. In some embodiments, the linker comprises G, S, A, and/or T residues. In some embodiments, the linker consists of glycine and serine residues. In some embodiments, the linker is between about 5 and about 20 amino acids or between about 5 and about 12 amino acids in length, e.g., about 5, about 6, about 7, about 8, about 9, about 10, about 11, about 12, about 13, about 14, about 15, about 16, about 17, about 18, about 19, or about 20 amino acids in length. In some embodiments, the linker comprises the sequence GGSGGGGSGG (SEQ ID NO:39). In some embodiments, the linker of the RNA vaccine comprises the sequence GGCGGCUCUGGAGGAGGCGGCUCCGGAGGC (SEQ ID NO:37). In some embodiments, the linker of the RNA vaccine is encoded by DNA comprising the sequence GGCGGCTCTGGAGGAGGCGGCTCCGGAGGC (SEQ ID NO:38).

In some embodiments, the RNA vaccine comprises a 5′ cap. The basic mRNA cap structure is known to contain a 5′-5′ triphosphate linkage between 2 nucleosides (e.g., two guanines) and a 7-methyl group on the distal guanine, i.e., m⁷GpppG. Exemplary cap structures can be found, e.g., in U.S. Pat. Nos. 8,153,773 and 9,295,717 and Kuhn, A. N. et al. (2010) Gene Ther. 17:961-971. In some embodiments, the 5′ cap has the structure m₂^{7,2′-O} GppₛpG. In some embodiments, the 5′ cap is a beta-S-ARCA cap. The S-ARCA cap structure includes a 2′-O methyl substitution (e.g., at the C2′ position of the m⁷G) and an S-substitution at one or more of the phosphate groups. In some embodiments, the 5′ cap comprises the structure:

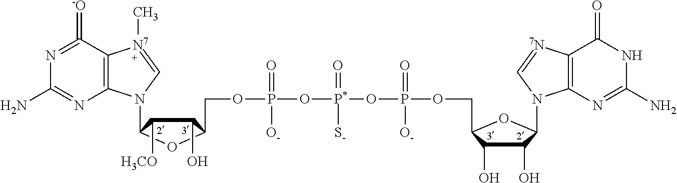

In some embodiments, the 5′ cap is the D1 diastereoisomer of beta-S-ARCA (see, e.g., U.S. Pat. No. 9,295,717). The * in the above structure indicates a stereogenic P center, which can exist in two diastereoisomers (designated D1 and D2). The D1 diastereomer of beta-S-ARCA or beta-S-ARCA(D1) is the diastereomer of beta-S-ARCA which elutes first on an HPLC column compared to the D2 diastereomer of beta-S-ARCA (beta-S-ARCA(D2)) and thus exhibits a shorter retention time. The HPLC preferably is an analytical HPLC. In one embodiment, a Supelcosil LC-18-T RP column, preferably of the format: 5 μm, 4.6×250 mm is used for separation, whereby a flow rate of 1.3 ml/min can be applied. In one embodiment, a gradient of methanol in ammonium acetate, for example, a 0-25% linear gradient of methanol in 0.05 M ammonium acetate, pH=5.9, within 15 min is used. UV-detection (VWD) can be performed at 260 nm and fluorescence detection (FLD) can be performed with excitation at 280 nm and detection at 337 nm.

In some embodiments, the RNA vaccine comprises a 5′ UTR. Certain untranslated sequences found 5′ to protein-coding sequences in mRNAs have been shown to increase translational efficiency. See, e.g., Kozak, M. (1987) J. Mol. Biol. 196:947-950. In some embodiments, the 5′ UTR comprises sequence from the human alpha globin mRNA. In some embodiments, the RNA vaccine comprises a 5′ UTR sequence of UUCUUCUGGUCCCCACAGACUCAGAGAGAACCCGCCACC (SEQ ID NO:23). In some embodiments, the 5′ UTR sequence of the RNA vaccine is encoded by DNA comprising the sequence TTCTTCTGGTCCCCACAGACTCAGAGAGAACCCGCCACC (SEQ ID NO:24). In some embodiments, the 5′ UTR sequence of RNA vaccine comprises the sequence GGCGAACUAGUAUUCUUCUGGUCCCCACAGACUCAGAGAGAACCCGCCACC (SEQ ID NO:21). In some embodiments, the 5′ UTR sequence of RNA vaccine is encoded by DNA comprising the sequence

(SEQ ID NO: 22)
GGCGAACTAGTATTCTTCTGGTCCCCACAGACTCAGAGAGAACCCGCCA
CC.

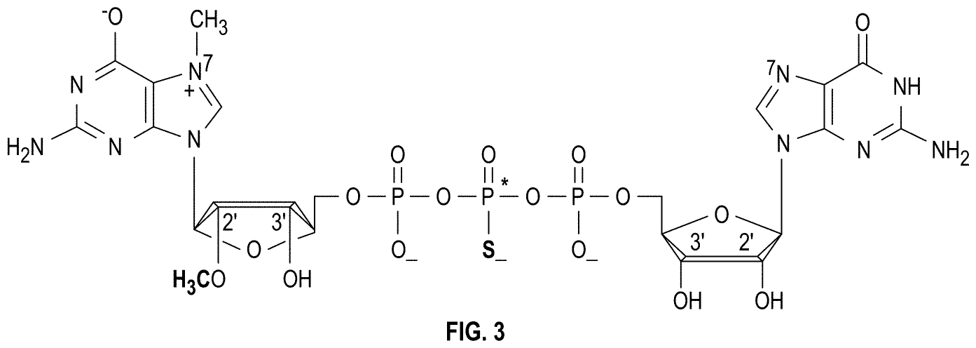

In some embodiments of the methods provided herein, the constant region of an exemplary RNA vaccine comprises the ribonucleotide sequence (5′->3′) of SEQ ID NO: 42. The linkage between the first two G residues is the unusual bond (5′ [symbol] 5′)-ppₛp-, e.g., as shown in Table 1 and in FIG. 3 for the 5′ capping structure. “N” refers to the position of polynucleotide sequence(s) encoding one or more (e.g., 1-20) neoepitopes (separated by optional linkers). The insertion site for tumor-specific sequences (C131-A132; marked in bold text) is depicted in bold text. See Table 1 for the modified bases and uncommon links in the exemplary RNA sequence.

TABLE 1

| Type | Location | Description |
|---|---|---|
| Modified Base | G1 | m₂^{7·2′·O} G |
| Uncommon Link | G1-G2 | (5′ [symbol] 5′)-ppₛp- |
| Uncommon Link | C131-A132 | Insertion site for |
| | | tumor-specific sequences |

In some embodiments, the RNA vaccine comprises a polynucleotide sequence encoding a secretory signal peptide. As is known in the art, a secretory signal peptide is an amino acid sequence that directs a polypeptide to be trafficked from the endoplasmic reticulum and into the secretory pathway upon translation. In some embodiments, the signal peptide is derived from a human polypeptide, such as an MHC polypeptide. See, e.g., Kreiter, S. et al. (2008) J. Immunol. 180:309-318, which describes an exemplary secretory signal peptide that improves processing and presentation of MHC Class I and II epitopes in human dendritic cells. In some embodiments, upon translation, the signal peptide is N-terminal to one or more neoepitope sequence(s) encoded by the RNA vaccine. In some embodiments, the secretory signal peptide comprises the sequence MRVMAPRTLILLLSGALALTETWAGS (SEQ ID NO:27). In some embodiments, the secretory signal peptide of the RNA vaccine comprises the sequence AUGAGAGUGAUGGCCCCCAGAACCCUGAUCCUGCUGCUGUCUGGCGCCCUGGCCCUGA CAGAGACAUGGGCCGGAAGC (SEQ ID NO:25). In some embodiments, the secretory signal peptide of the RNA vaccine is encoded by DNA comprising the sequence

(SEQ ID NO: 26)
ATGAGAGTGATGGCCCCCAGAACCCTGATCCTGCTGCTGTCTGGCGCCC
TGGCCCTGACAGAGACATGGGCCGGAAGC.

In some embodiments, the RNA vaccine comprises a polynucleotide sequence encoding at least a portion of a transmembrane and/or cytoplasmic domain. In some embodiments, the transmembrane and/or cytoplasmic domains are from the transmembrane/cytoplasmic domains of an MHC molecule. The term “major histocompatibility complex” and the abbreviation “MHC” relate to a complex of genes which occurs in all vertebrates. The function of MHC proteins or molecules in signaling between lymphocytes and antigen-presenting cells in normal immune responses involves them binding peptides and presenting them for possible recognition by T-cell receptors (TCR). MHC molecules bind peptides in an intracellular processing compartment and present these peptides on the surface of antigen-presenting cells to T cells. The human MHC region, also referred to as HLA, is located on chromosome 6 and comprises the class I region and the class II region. The class I alpha chains are glycoproteins having a molecular weight of about 44 kDa. The polypeptide chain has a length of somewhat more than 350 amino acid residues. It can be divided into three functional regions: an external, a transmembrane and a cytoplasmic region. The external region has a length of 283 amino acid residues and is divided into three domains, alpha1, alpha2 and alpha3. The domains and regions are usually encoded by separate exons of the class I gene. The transmembrane region spans the lipid bilayer of the plasma membrane. It consists of 23 usually hydrophobic amino acid residues which are arranged in an alpha helix. The cytoplasmic region, i.e. the part which faces the cytoplasm and which is connected to the transmembrane region, typically has a length of 32 amino acid residues and is able to interact with the elements of the cytoskeleton. The alpha chain interacts with beta2-microglobulin and thus forms alpha-beta2 dimers on the cell surface. The term “MHC class II” or “class II” relates to the major histocompatibility complex class II proteins or genes. Within the human MHC class II region there are the DP, DQ and DR subregions for class II alpha chain genes and beta chain genes (i.e. DPalpha, DPbeta, DQalpha, DQbeta, DRalpha and DRbeta). Class II molecules are heterodimers each consisting of an alpha chain and a beta chain. Both chains are glycoproteins having a molecular weight of 31-34 kDa (a) or 26-29 kDA (beta). The total length of the alpha chains varies from 229 to 233 amino acid residues, and that of the beta chains from 225 to 238 residues. Both alpha and beta chains consist of an external region, a connecting peptide, a transmembrane region and a cytoplasmic tail. The external region consists of two domains, alpha1 and alpha2 or beta1 and beta2. The connecting peptide is respectively beta and 9 residues long in alpha and beta chains. It connects the two domains to the transmembrane region which consists of 23 amino acid residues both in alpha chains and in beta chains. The length of the cytoplasmic region, i.e. the part which faces the cytoplasm and which is connected to the transmembrane region, varies from 3 to 16 residues in alpha chains and from 8 to 20 residues in beta chains. Exemplary transmembrane/cytoplasmic domain sequences are described in U.S. Pat. Nos. 8,178,653 and 8,637,006. In some embodiments, upon translation, the transmembrane and/or cytoplasmic domain is C-terminal to one or more neoepitope sequence(s) encoded by the RNA vaccine. In some embodiments, the transmembrane and/or cytoplasmic domain of the MHC molecule encoded by the RNA vaccine comprises the sequence IVGIVAGLAVLAVVVIGAVVATVMCRRKSSGGKGGSYSQAASSDSAQGSDVSLTA (SEQ ID NO:30). In some embodiments, the transmembrane and/or cytoplasmic domain of the MHC molecule comprises the sequence AUCGUGGGAAUUGUGGCAGGACUGGCAGUGCUGGCCGUGGUGGUGAUCGGAGCCGUG GUGGCUACCGUGAUGUGCAGACGGAAGUCCAGCGGAGGCAAGGGCGGCAGCUACAGC CAGGCCGCCAGCUCUGAUAGCGCCCAGGGCAGCGACGUGUCACUGACAGCC (SEQ ID NO:28). In some embodiments, the transmembrane and/or cytoplasmic domain of the MHC molecule is encoded by DNA comprising the sequence

(SEQ ID NO: 29)
ATCGTGGGAATTGTGGCAGGACTGGCAGTGCTGGCCGTGGTGGTGATCG
GAGCCGTGGTGGCTACCGTGATGTGCAGACGGAAGTCCAGCGGAGGCAA
GGGCGGCAGCTACAGCCAGGCCGCCAGCTCTGATAGCGCCCAGGGCAGC
GACGTGTCACTGACAGCC.

In some embodiments, the RNA vaccine comprises both a polynucleotide sequence encoding a secretory signal peptide that is N-terminal to the one or more neoepitope sequence(s) and a polynucleotide sequence encoding a transmembrane and/or cytoplasmic domain that is C-terminal to the one or more neoepitope sequence(s). Combining such sequences has been shown to improve processing and presentation of MHC Class I and II epitopes in human dendritic cells. See, e.g., Kreiter, S. et al. (2008) J. Immunol. 180:309-318.

In myeloid DCs, the RNA is released into the cytosol and translated into a poly-neoepitopic peptide. The polypeptide contains additional sequences to enhance antigen presentation. In some embodiments, a signal sequence (sec) from the MHCI heavy chain at the N-terminal of the polypeptide is used to target the nascent molecule to the endoplasmic reticulum, which has been shown to enhance MHCI presentation efficiency. Without wishing to be bound by theory, it is believed that the transmembrane and cytoplasmic domains of MHCI heavy chain guide the polypeptide to the endosomal/lysosomal compartments that were shown to improve MHCII presentation.

In some embodiments, the RNA vaccine comprises a 3′ UTR. Certain untranslated sequences found 3′ to protein-coding sequences in mRNAs have been shown to improve RNA stability, translation, and protein expression. Polynucleotide sequences suitable for use as 3′ UTRs are described, for example, in PG Pub. No. US20190071682. In some embodiments, the 3′ UTR comprises the 3′ untranslated region of AES or a fragment thereof and/or the non-coding RNA of the mitochondrially encoded 12S RNA. The term “AES” relates to Amino-Terminal Enhancer Of Split and includes the AES gene (see, e.g., NCBI Gene ID:166). The protein encoded by this gene belongs to the groucho/TLE family of proteins, can function as a homooligomer or as a heteroologimer with other family members to dominantly repress the expression of other family member genes. An exemplary AES mRNA sequence is provided in NCBI Ref. Seq. Accession NO. NM_198969. The term “MT_RNR1” relates to Mitochondrially Encoded 12S RNA and includes the MT_RNR1 gene (see, e.g., NCBI Gene ID:4549). This RNA gene belongs to the Mt_rRNA class. Diseases associated with MT-RNR1 include restrictive cardiomyopathy and auditory neuropathy. Among its related pathways are Ribosome biogenesis in eukaryotes and CFTR translational fidelity (class I mutations). An exemplary MT_RNR1 RNA sequence is present within the sequence of NCBI Ref. Seq. Accession NO. NC_012920. In some embodiments, the 3′ UTR of the RNA vaccine comprises the sequence CUGGUACUGCAUGCACGCAAUGCUAGCUGCCCCUUUCCCGUCCUGGGUACCCCGAGUC UCCCCCGACCUCGGGUCCCAGGUAUGCUCCCACCUCCACCUGCCCCACUCACCACCUCU GCUAGUUCCAGACACCUCC (SEQ ID NO:33). In some embodiments, the 3′ UTR of the RNA vaccine comprises the sequence CAAGCACGCAGCAAUGCAGCUCAAAACGCUUAGCCUAGCCACACCCCCACGGGAAACA GCAGUGAUUAACCUUUAGCAAUAAACGAAAGUUUAACUAAGCUAUACUAACCCCAGG GUUGGUCAAUUUCGUGCCAGCCACACCG (SEQ ID NO:35). In some embodiments, the 3′ UTR of the RNA vaccine comprises the sequence CUGGUACUGCAUGCACGCAAUGCUAGCUGCCCCUUUCCCGUCCUGGGUACCCCGAGUC UCCCCCGACCUCGGGUCCCAGGUAUGCUCCCACCUCCACCUGCCCCACUCACCACCUCU GCUAGUUCCAGACACCUCC (SEQ ID NO:33) and the sequence CAAGCACGCAGCAAUGCAGCUCAAAACGCUUAGCCUAGCCACACCCCCACGGGAAACA GCAGUGAUUAACCUUUAGCAAUAAACGAAAGUUUAACUAAGCUAUACUAACCCCAGG GUUGGUCAAUUUCGUGCCAGCCACACCG (SEQ ID NO:35). In some embodiments, the 3′ UTR of the RNA vaccine comprises the sequence CUCGAGCUGGUACUGCAUGCACGCAAUGCUAGCUGCCCCUUUCCCGUCCUGGGUACCC CGAGUCUCCCCCGACCUCGGGUCCCAGGUAUGCUCCCACCUCCACCUGCCCCACUCACC ACCUCUGCUAGUUCCAGACACCUCCCAAGCACGCAGCAAUGCAGCUCAAAACGCUUAG CCUAGCCACACCCCCACGGGAAACAGCAGUGAUUAACCUUUAGCAAUAAACGAAAGUU UAACUAAGCUAUACUAACCCCAGGGUUGGUCAAUUUCGUGCCAGCCACACCGAGACCU GGUCCAGAGUCGCUAGCCGCGUCGCU (SEQ ID NO:31). In some embodiments, the 3′ UTR of the RNA vaccine is encoded by DNA comprising the sequence CTGGTACTGCATGCACGCAATGCTAGCTGCCCCTTTCCCGTCCTGGGTACCCCGAGTCTC CCCCGACCTCGGGTCCCAGGTATGCTCCCACCTCCACCTGCCCCACTCACCACCTCTGCT AGTTCCAGACACCTCC (SEQ ID NO:34). In some embodiments, the 3′ UTR of the RNA vaccine is encoded by DNA comprising the sequence CAAGCACGCAGCAATGCAGCTCAAAACGCTTAGCCTAGCCACACCCCCACGGGAAACAG CAGTGATTAACCTTTAGCAATAAACGAAAGTTTAACTAAGCTATACTAACCCCAGGGTTG GTCAATTTCGTGCCAGCCACACCG (SEQ ID NO:36). In some embodiments, the 3′ UTR of the RNA vaccine is encoded by DNA comprising the sequence CTGGTACTGCATGCACGCAATGCTAGCTGCCCCTTTCCCGTCCTGGGTACCCCGAGTCTC CCCCGACCTCGGGTCCCAGGTATGCTCCCACCTCCACCTGCCCCACTCACCACCTCTGCT AGTTCCAGACACCTCC (SEQ ID NO:34) and the sequence CAAGCACGCAGCAATGCAGCTCAAAACGCTTAGCCTAGCCACACCCCCACGGGAAACAG CAGTGATTAACCTTTAGCAATAAACGAAAGTTTAACTAAGCTATACTAACCCCAGGGTTG GTCAATTTCGTGCCAGCCACACCG (SEQ ID NO:36). In some embodiments, the 3′ UTR of the RNA vaccine is encoded by DNA comprising the sequence

(SEQ ID NO: 32)
CTGGTACTGCATGCACGCAATGCTAGCTGCCCCTTTCCCGTCCTGGGTA
CCCCGAGTCTCCCCCGACCTCGGGTCCCAGGTATGCTCCCACCTCCACC
TGCCCCACTCACCACCTCTGCTAGTTCCAGACACCTCCCAAGCACGCAG
CAATGCAGCTCAAAACGCTTAGCCTAGCCACACCCCCACGGGAAACAGC
AGTGATTAACCTTTAGCAATAAACGAAAGTTTAACTAAGCTATACTAAC
CCCAGGGTTGGTCAATTTCGTGCCAGCCACACCGAGACCTGGTCCAGAG
TCGCTAGCCGCGTCGCT.

In some embodiments, the RNA vaccine comprises a poly(A) tail at its 3′-end. In some embodiments, the poly(A) tail comprises more than 50 or more than 100 adenine nucleotides. For example, in some embodiments, the poly(A) tail comprises 120 adenine nucleotides. This poly(A) tail has been demonstrated to enhance RNA stability and translation efficiency (Holtkamp, S. et al. (2006) Blood 108:4009-4017). In some embodiments, the RNA comprising a poly(A) tail is generated by transcribing a DNA molecule comprising in the 5′→3′ direction of transcription, a polynucleotide sequence that encodes at least 50, 100, or 120 adenine consecutive nucleotides and a recognition sequence for a type IIS restriction endonuclease. Exemplary poly(A) tail and 3′ UTR sequences that improve translation are found, e.g., in U.S. Pat. No. 9,476,055.

In some embodiments, an RNA vaccine or molecule of the present disclosure comprises the general structure (in the 5′→3′ direction): (1) a 5′ cap; (2) a 5′ untranslated region (UTR); (3) a polynucleotide sequence encoding a secretory signal peptide; (4) a polynucleotide sequence encoding at least a portion of a transmembrane and cytoplasmic domain of a major histocompatibility complex (MHC) molecule; (5) a 3′ UTR comprising: (a) a 3′ untranslated region of an Amino-Terminal Enhancer of Split (AES) mRNA or a fragment thereof; and (b) non-coding RNA of a mitochondrially encoded 12S RNA or a fragment thereof; and (6) a poly(A) sequence. In some embodiments, an RNA vaccine or molecule of the present disclosure comprises, in the 5′→3′ direction: the polynucleotide sequence GGCGAACUAGUAUUCUUCUGGUCCCCACAGACUCAGAGAGAACCCGCCACCAUGAGAG UGAUGGCCCCCAGAACCCUGAUCCUGCUGCUGUCUGGCGCCCUGGCCCUGACAGAGAC AUGGGCCGGAAGC (SEQ ID NO:19); and the polynucleotide sequence AUCGUGGGAAUUGUGGCAGGACUGGCAGUGCUGGCCGUGGUGGUGAUCGGAGCCGUG GUGGCUACCGUGAUGUGCAGACGGAAGUCCAGCGGAGGCAAGGGCGGCAGCUACAGC CAGGCCGCCAGCUCUGAUAGCGCCCAGGGCAGCGACGUGUCACUGACAGCCUAGUAAC UCGAGCUGGUACUGCAUGCACGCAAUGCUAGCUGCCCCUUUCCCGUCCUGGGUACCCC GAGUCUCCCCCGACCUCGGGUCCCAGGUAUGCUCCCACCUCCACCUGCCCCACUCACCA CCUCUGCUAGUUCCAGACACCUCCCAAGCACGCAGCAAUGCAGCUCAAAACGCUUAGC CUAGCCACACCCCCACGGGAAACAGCAGUGAUUAACCUUUAGCAAUAAACGAAAGUUU AACUAAGCUAUACUAACCCCAGGGUUGGUCAAUUUCGUGCCAGCCACACCGAGACCUG GUCCAGAGUCGCUAGCCGCGUCGCU (SEQ ID NO:20). Advantageously, RNA vaccines comprising this combination and orientation of structures or sequences are characterized by one or more of: improved RNA stability, enhanced translational efficiency, improved antigen presentation and/or processing (e.g., by DCs), and increased protein expression.

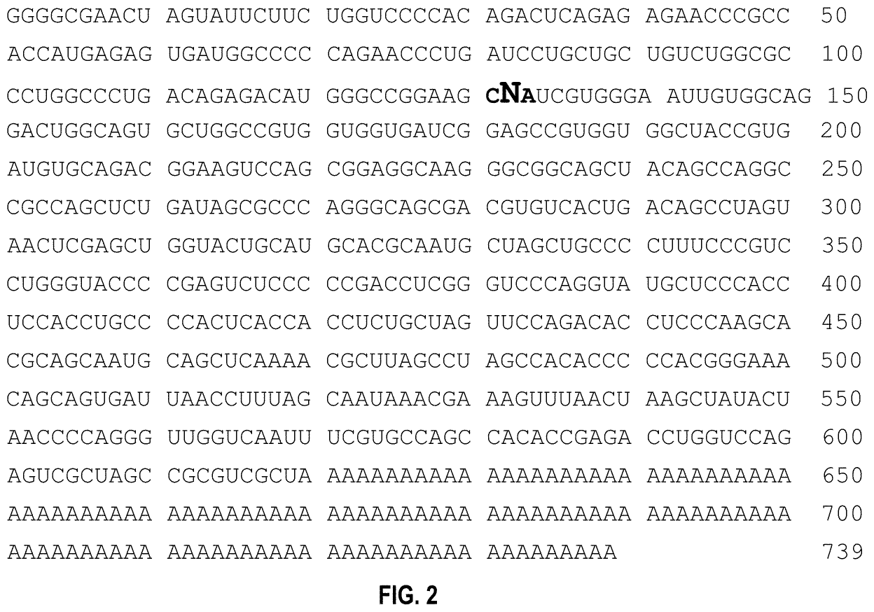

In some embodiments, an RNA vaccine or molecule of the present disclosure comprises the sequence (in the 5′→3′ direction) of SEQ ID NO:42. See, e.g., FIG. 2. In some embodiments, N refers to a polynucleotide sequence encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 different neoepitopes. In some embodiments, N refers to a polynucleotide sequence encoding one or more linker-epitope modules (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 different linker-epitope modules). In some embodiments, N refers to a polynucleotide sequence encoding one or more linker-epitope modules (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 different linker-epitope modules) and an additional amino acid linker at the 3′ end.

In some embodiments, the RNA vaccine or molecule further comprises a polynucleotide sequence encoding at least one neoepitopes; wherein the polynucleotide sequence encoding the at least one neoepitope is between the polynucleotide sequence encoding the secretory signal peptide and the polynucleotide sequence encoding the at least portion of the transmembrane and cytoplasmic domain of the MHC molecule in the 5′→3′ direction. In some embodiments, the RNA molecule comprises a polynucleotide sequence encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes.

In some embodiments, the RNA vaccine or molecule further comprises, in the 5′→3′ direction: a polynucleotide sequence encoding an amino acid linker; and a polynucleotide sequence encoding a neoepitope. In some embodiments, the polynucleotide sequences encoding the amino acid linker and the neoepitope form a linker-neoepitope module (e.g., a continuous sequence in the 5′→3′ direction in the same open-reading frame). In some embodiments, the polynucleotide sequences forming the linker-neoepitope module are between the polynucleotide sequence encoding the secretory signal peptide and the polynucleotide sequence encoding the at least portion of the transmembrane and cytoplasmic domain of the MHC molecule, or between the sequences of SEQ ID NO:19 and SEQ ID NO:20, in the 5′→3′ direction. In some embodiments, the RNA vaccine or molecule comprises 2, 3, 4, 5, 6, 7, 8, 9, 10, 11, 12, 13, 14, 15, 16, 17, 18, 19, 20, 21, 22, 23, 24, 25, 26, 27, 28, 28, 29, or 30 linker-epitope modules. In some embodiments, each of the linker-epitope modules encodes a different neoepitope. In some embodiments, the RNA vaccine or molecule comprises 2, 3, 4, 5, 6, 7, 8, 9, 10, 11, 12, 13, 14, 15, 16, 17, 18, 19, or 20 linker-epitope modules, and the RNA vaccine or molecule comprises polynucleotides encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes. In some embodiments, the RNA vaccine or molecule comprises 5, 10, or 20 linker-epitope modules. In some embodiments, each of the linker-epitope modules encodes a different neoepitope. In some embodiments, the linker-epitope modules form a continuous sequence in the 5′→3′ direction in the same open-reading frame. In some embodiments, the polynucleotide sequence encoding the linker of the first linker-epitope module is 3′ of the polynucleotide sequence encoding the secretory signal peptide. In some embodiments, the polynucleotide sequence encoding the neoepitope of the last linker-epitope module is 5′ of the polynucleotide sequence encoding the at least portion of the transmembrane and cytoplasmic domain of the MHC molecule.

In some embodiments, the RNA vaccine is at least 800 nucleotides, at least 1000 nucleotides, or at least 1200 nucleotides in length. In some embodiments, the RNA vaccine is less than 2000 nucleotides in length. In some embodiments, the RNA vaccine is at least 800 nucleotides but less than 2000 nucleotides in length, at least 1000 nucleotides but less than 2000 nucleotides in length, at least 1200 nucleotides but less than 2000 nucleotides in length, at least 1400 nucleotides but less than 2000 nucleotides in length, at least 800 nucleotides but less than 1400 nucleotides in length, or at least 800 nucleotides but less than 2000 nucleotides in length. For example, the constant regions of an RNA vaccine comprising the elements described above are approximately 800 nucleotides in length. In some embodiments, an RNA vaccine comprising 5 tumor-specific neoepitopes (e.g., each encoding 27 amino acids) is greater than 1300 nucleotides in length. In some embodiments, an RNA vaccine comprising 10 tumor-specific neoepitopes (e.g., each encoding 27 amino acids) is greater than 1800 nucleotides in length.

In some embodiments, the RNA vaccine is formulated in a lipoplex nanoparticle or liposome. In some embodiments, a lipoplex nanoparticle formulation for the RNA (RNA-Lipoplex) is used to enable IV delivery of an RNA vaccine of the present disclosure. In some embodiments, a lipoplex nanoparticle formulation for the RNA cancer vaccine comprising the synthetic cationic lipid (R)-N,N,N-trimethyl-2,3-dioleyloxy-1-propanaminium chloride (DOTMA) and the phospholipid 1,2-dioleoyl-sn-glycero-3-phosphoethanolamine (DOPE) is used, e.g., to enable IV delivery. The DOTMA/DOPE liposomal component has been optimized for IV delivery and targeting of antigen-presenting cells in the spleen and other lymphoid organs.

In one embodiment, the nanoparticles comprise at least one lipid. In one embodiment, the nanoparticles comprise at least one cationic lipid. The cationic lipid can be monocationic or polycationic. Any cationic amphiphilic molecule, e.g., a molecule which comprises at least one hydrophilic and lipophilic moiety is a cationic lipid within the meaning of the present invention. In one embodiment, the positive charges are contributed by the at least one cationic lipid and the negative charges are contributed by the RNA. In one embodiment, the nanoparticles comprises at least one helper lipid. The helper lipid may be a neutral or an anionic lipid. The helper lipid may be a natural lipid, such as a phospholipid or an analogue of a natural lipid, or a fully synthetic lipid, or lipid-like molecule, with no similarities with natural lipids. In one embodiment, the cationic lipid and/or the helper lipid is a bilayer forming lipid.

In one embodiment, the at least one cationic lipid comprises 1,2-di-O-octadecenyl-3-trimethylammonium propane (DOTMA) or analogs or derivatives thereof and/or 1,2-dioleoyl-3-trimethylammonium-propane (DOTAP) or analogs or derivatives thereof.

In one embodiment, the at least one helper lipid comprises 1,2-di-(9Z-octadecenoyl)-sn-glycero-3-phosphoethanolamine (DOPE) or analogs or derivatives thereof, cholesterol (Chol) or analogs or derivatives thereof and/or 1,2-dioleoyl-sn-glycero-3-phosphocholine (DOPC) or analogs or derivatives thereof.

In one embodiment, the molar ratio of the at least one cationic lipid to the at least one helper lipid is from 10:0 to 3:7, preferably 9:1 to 3:7, 4:1 to 1:2, 4:1 to 2:3, 7:3 to 1:1, or 2:1 to 1:1, preferably about 1:1. In one embodiment, in this ratio, the molar amount of the cationic lipid results from the molar amount of the cationic lipid multiplied by the number of positive charges in the cationic lipid.

In one embodiment, the lipid is comprised in a vesicle encapsulating said RNA. The vesicle may be a multilamellar vesicle, an unilamellar vesicle, or a mixture thereof. The vesicle may be a liposome.

Nanoparticles or liposomes described herein can be formed by adjusting a positive to negative charge, depending on the (+/−) charge ratio of a cationic lipid to RNA and mixing the RNA and the cationic lipid. The +/− charge ratio of the cationic lipid to the RNA in the nanoparticles described herein can be calculated by the following equation. (+/− charge ratio)=[(cationic lipid amount (mol))*(the total number of positive charges in the cationic lipid)]:[(RNA amount (mol))*(the total number of negative charges in RNA)]. The RNA amount and the cationic lipid amount can be easily determined by one skilled in the art in view of a loading amount upon preparation of the nanoparticles. For further descriptions of exemplary nanoparticles, see, e.g., PG Pub. No. US20150086612.

In one embodiment, the overall charge ratio of positive charges to negative charges in the nanoparticles or liposomes (e.g., at physiological pH) is between 1.4:1 and 1:8, preferably between 1.2:1 and 1:4, e.g. between 1:1 and 1:3 such as between 1:1.2 and 1:2, 1:1.2 and 1:1.8, 1:1.3 and 1:1.7, in particular between 1:1.4 and 1:1.6, such as about 1:1.5. In some embodiments, at physiological pH the overall charge ratio of positive charges to negative charges of the nanoparticles is between 1:1.2 (0.83) and 1:2 (0.5). In some embodiments, at physiological pH the overall charge ratio of positive charges to negative charges of the nanoparticles or liposomes is between 1.6:2 (0.8) and 1:2 (0.5) or between 1.6:2 (0.8) and 1.1:2 (0.55). In some embodiments, at physiological pH the overall charge ratio of positive charges to negative charges of the nanoparticles or liposomes is 1.3:2 (0.65). In some embodiments, at physiological pH the overall charge ratio of positive charges to negative charges of the liposome is not lower than 1.0:2.0. In some embodiments, at physiological pH the overall charge ratio of positive charges to negative charges of the liposome is not higher than 1.9:2.0. In some embodiments, at physiological pH the overall charge ratio of positive charges to negative charges of the liposome is not lower than 1.0:2.0 and not higher than 1.9:2.0.

In one embodiment, the nanoparticles are lipoplexes comprising DOTMA and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In one embodiment, the nanoparticles are lipoplexes comprising DOTMA and Cholesterol in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In one embodiment, the nanoparticles are lipoplexes comprising DOTAP and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In one embodiment, the nanoparticles are lipoplexes comprising DOTMA and DOPE in a molar ratio of 2:1 to 1:2, preferably 2:1 to 1:1, and wherein the charge ratio of positive charges in DOTMA to negative charges in the RNA is 1.4:1 or less. In one embodiment, the nanoparticles are lipoplexes comprising DOTMA and cholesterol in a molar ratio of 2:1 to 1:2, preferably 2:1 to 1:1, and wherein the charge ratio of positive charges in DOTMA to negative charges in the RNA is 1.4:1 or less. In one embodiment, the nanoparticles are lipoplexes comprising DOTAP and DOPE in a molar ratio of 2:1 to 1:2, preferably 2:1 to 1:1, and wherein the charge ratio of positive charges in DOTAP to negative charges in the RNA is 1.4:1 or less.

In one embodiment, the zeta potential of the nanoparticles or liposomes is −5 or less, −10 or less, −15 or less, −20 or less or −25 or less. In various embodiments, the zeta potential of the nanoparticles or liposomes is −35 or higher, −30 or higher or −25 or higher. In one embodiment, the nanoparticles or liposomes have a zeta potential from 0 mV to −50 mV, preferably 0 mV to −40 mV or −10 mV to −30 mV.

In some embodiments, the polydispersity index of the nanoparticles or liposomes is 0.5 or less, 0.4 or less, or 0.3 or less, as measured by dynamic light scattering.

In some embodiments, the nanoparticles or liposomes have an average diameter in the range of about 50 nm to about 1000 nm, from about 100 nm to about 800 nm, from about 200 nm to about 600 nm, from about 250 nm to about 700 nm, or from about 250 nm to about 550 nm, as measured by dynamic light scattering.

In some embodiments, the PCV is administered intravenously, for example, in a liposomal formulation, at doses of 15 μg, 25 μg, 38 μg, 50 μg, or 100 μg. In some embodiments, 15 μg, 25 μg, 38 μg, 50 μg, or 100 μg of RNA is delivered per dose (i.e., dose weight reflects the weight of RNA administered, not the total weight of the formulation or lipoplex administered). More than one PCV may be administered to a subject, e.g., subject is administered one PCV with a combination of neoepitopes and also administered a separate PCV with a different combination of neoepitopes. In some embodiments, a first PCV with ten neoepitopes is administered in combination with a second PCV with ten alternative epitopes.

In some embodiments, the PCV is administered such that it is delivered to the spleen. For example, the PCV can be administered such that one or more antigen(s) (e.g., tumor-specific neoantigens) are delivered to antigen presenting cells (e.g., in the spleen).

Any of the PCVs or RNA vaccines of the present disclosure may find use in the methods described herein. For example, in some embodiments, a PD-1 axis binding antagonist of the present disclosure is administered in combination with a personalized cancer vaccine (PCV), e.g., an RNA vaccine described herein.

Further provided herein are DNA molecules encoding any of the RNA vaccines of the present disclosure. For example, in some embodiments, a DNA molecule of the present disclosure comprises the general structure (in the 5′→3′ direction): (1) a polynucleotide sequence encoding a 5′ untranslated region (UTR); (2) a polynucleotide sequence encoding a secretory signal peptide; (3) a polynucleotide sequence encoding at least a portion of a transmembrane and cytoplasmic domain of a major histocompatibility complex (MHC) molecule; (4) a polynucleotide sequence encoding a 3′ UTR comprising: (a) a 3′ untranslated region of an Amino-Terminal Enhancer of Split (AES) mRNA or a fragment thereof; and (b) non-coding RNA of a mitochondrially encoded 12S RNA or a fragment thereof; and (5) a polynucleotide sequence encoding a poly(A) sequence. In some embodiments, a DNA molecule of the present disclosure comprises, in the 5′→3′ direction: the polynucleotide sequence

(SEQ ID NO: 40)
GGCGAACTAGTATTCTTCTGGTCCCCACAGACTCAGAGAGAACCCGCCA
CCATGAGAGTGATGGCCCCCAGAACCCTGATCCTGCTGCTGTCTGGCGC
CCTGGCCCTGACAGAGACATGGGCCGGAAGC;
and the polynucleotide sequence
(SEQ ID NO: 41)
ATCGTGGGAATTGTGGCAGGACTGGCAGTGCTGGCCGTGGTGGTGATCG
GAGCCGTGGTGGCTACCGTGATGTGCAGACGGAAGTCCAGCGGAGGCAA
GGGCGGCAGCTACAGCCAGGCCGCCAGCTCTGATAGCGCCCAGGGCAGC
GACGTGTCACTGACAGCCTAGTAACTCGAGCTGGTACTGCATGCACGCA
ATGCTAGCTGCCCCTTTCCCGTCCTGGGTACCCCGAGTCTCCCCCGACC
TCGGGTCCCAGGTATGCTCCCACCTCCACCTGCCCCACTCACCACCTCT
GCTAGTTCCAGACACCTCCCAAGCACGCAGCAATGCAGCTCAAAACGCT
TAGCCTAGCCACACCCCCACGGGAAACAGCAGTGATTAACCTTTAGCAA
TAAACGAAAGTTTAACTAAGCTATACTAACCCCAGGGTTGGTCAATTTC
GTGCCAGCCACACCGAGACCTGGTCCAGAGTCGCTAGCCGCGTCGCT.

In some embodiments, the DNA molecule further comprises, in the 5′→3′ direction: a polynucleotide sequence encoding an amino acid linker; and a polynucleotide sequence encoding a neoepitope. In some embodiments, the polynucleotide sequences encoding the amino acid linker and the neoepitope form a linker-neoepitope module (e.g., a continuous sequence in the 5′→3′ direction in the same open-reading frame). In some embodiments, the polynucleotide sequences forming the linker-neoepitope module are between the polynucleotide sequence encoding the secretory signal peptide and the polynucleotide sequence encoding the at least portion of the transmembrane and cytoplasmic domain of the MHC molecule, or between the sequences of SEQ ID NO:40 and SEQ ID NO:41, in the 5′→3′ direction. In some embodiments, the DNA molecule comprises 2, 3, 4, 5, 6, 7, 8, 9, 10, 11, 12, 13, 14, 15, 16, 17, 18, 19, 20, 21, 22, 23, 24, 25, 26, 27, 28, 28, 29, or 30 linker-epitope modules, and each of the linker-epitope modules encodes a different neoepitope. In some embodiments, the DNA molecule comprises 2, 3, 4, 5, 6, 7, 8, 9, 10, 11, 12, 13, 14, 15, 16, 17, 18, 19, or 20 linker-epitope modules, and the DNA molecule comprises polynucleotides encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes. In some embodiments, the DNA molecule comprises 5, 10, or 20 linker-epitope modules. In some embodiments, each of the linker-epitope modules encodes a different neoepitope. In some embodiments, the linker-epitope modules form a continuous sequence in the 5′→3′ direction in the same open-reading frame. In some embodiments, the polynucleotide sequence encoding the linker of the first linker-epitope module is 3′ of the polynucleotide sequence encoding the secretory signal peptide. In some embodiments, the polynucleotide sequence encoding the neoepitope of the last linker-epitope module is 5′ of the polynucleotide sequence encoding the at least portion of the transmembrane and cytoplasmic domain of the MHC molecule.

Also provided herein are methods of producing any of the RNA vaccine of the present disclosure, comprising transcribing (e.g., by transcription of linear, double-stranded DNA or plasmid DNA, such as by in vitro transcription) a DNA molecule of the present disclosure. In some embodiments, the methods further comprise isolating and/or purifying the transcribed RNA molecule from the DNA molecule.

In some embodiments, an RNA or DNA molecule of the present disclosure comprises a type HS restriction cleavage site, which allows RNA to be transcribed under the control of a 5′ RNA polymerase promoter and which contains a polyadenyl cassette (poly(A) sequence), wherein the recognition sequence is located 3′ of the poly(A) sequence, while the cleavage site is located upstream and thus within the poly(A) sequence. Restriction cleavage at the type HS restriction cleavage site enables a plasmid to be linearized within the poly(A) sequence, as described in U.S. Pat. Nos. 9,476,055 and 10,106,800. The linearized plasmid can then be used as template for in vitro transcription, the resulting transcript ending in an unmasked poly(A) sequence. Any of the type HS restriction cleavage sites described in U.S. Pat. Nos. 9,476,055 and 10,106,800 may be used.

In some embodiments of the methods provided herein, the RNA vaccine includes one or more polynucleotides encoding 10-20 (e.g., any of 10, 11, 12, 13, 14, 15, 16, 17, 18, 19, or 20) neoepitopes resulting from cancer-specific somatic mutations present in the tumor specimen. In certain embodiments, the RNA vaccine is formulated in a lipoplex nanoparticle or liposome. In certain embodiments, the lipoplex nanoparticle or liposome includes one or more lipids that form a multilamellar structure that encapsulates the RNA of the RNA vaccine. In certain embodiments, the one or more lipids include at least one cationic lipid and at least one helper lipid. In certain embodiments, the one or more lipids include (R)-N,N,N-trimethyl-2,3-dioleyloxy-1-propanaminium chloride (DOTMA) and 1,2-dioleoyl-sn-glycero-3-phosphoethanolamine (DOPE). In certain embodiments, at physiological pH the overall charge ratio of positive charges to negative charges of the liposome is 1.3:2 (0.65).

In certain embodiments, the RNA vaccine includes an RNA molecule including, in the 5′→3′ direction: (1) a 5′ cap; (2) a 5′ untranslated region (UTR); (3) a polynucleotide sequence encoding a secretory signal peptide; (4) a polynucleotide sequence encoding the one or more neoepitopes resulting from cancer-specific somatic mutations present in the tumor specimen; (5) a polynucleotide sequence encoding at least a portion of a transmembrane and cytoplasmic domain of a major histocompatibility complex (MHC) molecule; (6) a 3′ UTR including: (a) a 3′ untranslated region of an Amino-Terminal Enhancer of Split (AES) mRNA or a fragment thereof; and (b) non-coding RNA of a mitochondrially encoded 12S RNA or a fragment thereof; and (7) a poly(A) sequence.

In certain embodiments, the RNA molecule further includes a polynucleotide sequence encoding an amino acid linker; wherein the polynucleotide sequences encoding the amino acid linker and a first of the one or more neoepitopes form a first linker-neoepitope module; and wherein the polynucleotide sequences forming the first linker-neoepitope module are between the polynucleotide sequence encoding the secretory signal peptide and the polynucleotide sequence encoding the at least portion of the transmembrane and cytoplasmic domain of the MHC molecule in the 5′→3′ direction. In certain embodiments, the amino acid linker includes the sequence GGSGGGGSGG (SEQ ID NO: 39). In certain embodiments, the polynucleotide sequence encoding the amino acid linker includes the sequence GGCGGCUCUGGAGGAGGCGGCUCCGGAGGC (SEQ ID NO: 37).

In certain embodiments, the RNA molecule further includes, in the 5′→3′ direction: at least a second linker-epitope module, wherein the at least second linker-epitope module includes a polynucleotide sequence encoding an amino acid linker and a polynucleotide sequence encoding a neoepitope; wherein the polynucleotide sequences forming the second linker-neoepitope module are between the polynucleotide sequence encoding the neoepitope of the first linker-neoepitope module and the polynucleotide sequence encoding the at least portion of the transmembrane and cytoplasmic domain of the MHC molecule in the 5′→3′ direction; and wherein the neoepitope of the first linker-epitope module is different from the neoepitope of the second linker-epitope module. In certain embodiments, the RNA molecule includes 5 linker-epitope modules, wherein the 5 linker-epitope modules each encode a different neoepitope. In certain embodiments, the RNA molecule includes 10 linker-epitope modules, wherein the 10 linker-epitope modules each encode a different neoepitope. In certain embodiments, the RNA molecule includes 20 linker-epitope modules, wherein the 20 linker-epitope modules each encode a different neoepitope.

In certain embodiments, the RNA molecule further includes a second polynucleotide sequence encoding an amino acid linker, wherein the second polynucleotide sequence encoding the amino acid linker is between the polynucleotide sequence encoding the neoepitope that is most distal in the 3′ direction and the polynucleotide sequence encoding the at least portion of the transmembrane and cytoplasmic domain of the MHC molecule.

In certain embodiments, the 5′ cap includes a D1 diastereoisomer of the structure:

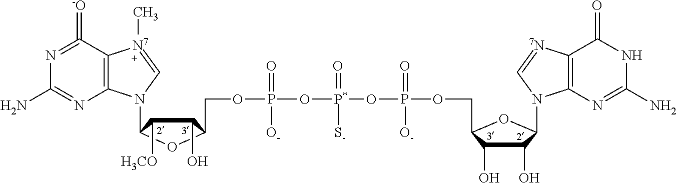

In certain embodiments, the 5′ UTR includes the sequence UUCUUCUGGUCCCCACAGACUCAGAGAGAACCCGCCACC (SEQ ID NO:23). In certain embodiments, the 5′ UTR includes the sequence

(SEQ ID NO: 21)
GGCGAACUAGUAUUCUUCUGGUCCCCACAGACUCAGAGAGAACCCGCCA
CC.

In certain embodiments, the secretory signal peptide includes the amino acid sequence MRVMAPRTLILLLSGALALTETWAGS (SEQ ID NO:27). In certain embodiments, the polynucleotide sequence encoding the secretory signal peptide includes the sequence

(SEO ID NO: 25)
AUGAGAGUGAUGGCCCCCAGAACCCUGAUCCUGCUGCUGUCUGGCGCCC
UGGCCCUGACAGAGACAUGGGCCGGAAGC

In certain embodiments, the at least portion of the transmembrane and cytoplasmic domain of the MHC molecule includes the amino acid sequence IVGIVAGLAVLAVVVIGAVVATVMCRRKSSGGKGGSYSQAASSDSAQGSDVSLTA (SEQ ID NO:30). In certain embodiments, the polynucleotide sequence encoding the at least portion of the transmembrane and cytoplasmic domain of the MHC molecule includes the sequence

(SEQ ID NO: 28)
AUCGUGGGAAUUGUGGCAGGACUGGCAGUGCUGGCCGUGGUGGUGAUCG
GAGCCGUGGUGGCUACCGUGAUGUGCAGACGGAAGUCCAGCGGAGGCAA
GGGCGGCAGCUACAGCCAGGCCGCCAGCUCUGAUAGCGCCCAGGGCAGC
GACGUGUCACUGACAGCC.

In certain embodiments, the 3′ untranslated region of the AES mRNA includes the sequence CUGGUACUGCAUGCACGCAAUGCUAGCUGCCCCUUUCCCGUCCUGGGUACCCCGAGUC UCCCCCGACCUCGGGUCCCAGGUAUGCUCCCACCUCCACCUGCCCCACUCACCACCUCU GCUAGUUCCAGACACCUCC (SEQ ID NO:33). In certain embodiments, the non-coding RNA of the mitochondrially encoded 12S RNA includes the sequence CAAGCACGCAGCAAUGCAGCUCAAAACGCUUAGCCUAGCCACACCCCCACGGGAAACA GCAGUGAUUAACCUUUAGCAAUAAACGAAAGUUUAACUAAGCUAUACUAACCCCAGG GUUGGUCAAUUUCGUGCCAGCCACACCG (SEQ ID NO:35). In certain embodiments, the 3′ UTR includes the sequence

(SEQ ID NO: 31)
CUCGAGCUGGUACUGCAUGCACGCAAUGCUAGCUGCCCCUUUCCCGUCC
UGGGUACCCCGAGUCUCCCCCGACCUCGGGUCCCAGGUAUGCUCCCACC
UCCACCUGCCCCACUCACCACCUCUGCUAGUUCCAGACACCUCCCAAGC
ACGCAGCAAUGCAGCUCAAAACGCUUAGCCUAGCCACACCCCCACGGGA
AACAGCAGUGAUUAACCUUUAGCAAUAAACGAAAGUUUAACUAAGCUAU
ACUAACCCCAGGGUUGGUCAAUUUCGUGCCAGCCACACCGAGACCUGGU
CCAGAGUCGCUAGCCGCGUCGCU.

In certain embodiments, the poly(A) sequence includes 120 adenine nucleotides.

In certain embodiments, the RNA vaccine includes an RNA molecule including, in the 5′→3′ direction: the polynucleotide sequence GGCGAACUAGUAUUCUUCUGGUCCCCACAGACUCAGAGAGAACCCGCCACCAUGAGAG UGAUGGCCCCCAGAACCCUGAUCCUGCUGCUGUCUGGCGCCCUGGCCCUGACAGAGAC AUGGGCCGGAAGC (SEQ ID NO:19); a polynucleotide sequence encoding the one or more neoepitopes resulting from cancer-specific somatic mutations present in the tumor specimen; and the polynucleotide sequence

(SEQ ID NO: 20)
AUCGUGGGAAUUGUGGCAGGACUGGCAGUGCUGGCCGUGGUGGUGAUCG
GAGCCGUGGUGGCUACCGUGAUGUGCAGACGGAAGUCCAGCGGAGGCAA
GGGCGGCAGCUACAGCCAGGCCGCCAGCUCUGAUAGCGCCCAGGGCAGC
GACGUGUCACUGACAGCCUAGUAACUCGAGCUGGUACUGCAUGCACGCA
AUGCUAGCUGCCCCUUUCCCGUCCUGGGUACCCCGAGUCUCCCCCGACC
UCGGGUCCCAGGUAUGCUCCCACCUCCACCUGCCCCACUCACCACCUCU
GCUAGUUCCAGACACCUCCCAAGCACGCAGCAAUGCAGCUCAAAACGCU
UAGCCUAGCCACACCCCCACGGGAAACAGCAGUGAUUAACCUUUAGCAA
UAAACGAAAGUUUAACUAAGCUAUACUAACCCCAGGGUUGGUCAAUUUC
GUGCCAGCCACACCGAGACCUGGUCCAGAGUCGCUAGCCGCGUCGCU.

### IV. PD-1 Axis Binding Antagonists

In some embodiments, a PCV (e.g., an RNA vaccine) of the present disclosure is administered in combination with a PD-1 axis binding antagonist.

For example, a PD-1 axis binding antagonist includes a PD-1 binding antagonist, a PDL1 binding antagonist and a PDL2 binding antagonist. Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 binding antagonist is a molecule that inhibits the binding of PD-1 to its ligand binding partner(s). In a specific aspect the PD-1 ligand binding partners are PDL1 and/or PDL2. In another embodiment, a PDL1 binding antagonist is a molecule that inhibits the binding of PDL1 to its binding partner(s). In a specific aspect, PDL1 binding partner(s) are PD-1 and/or B7-1. In another embodiment, the PDL2 binding antagonist is a molecule that inhibits the binding of PDL2 to its binding partner(s). In a specific aspect, a PDL2 binding partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody).

In some embodiments, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO2006/121168. In some embodiments, the anti-PD-1 antibody comprises a heavy chain and a light chain sequence, wherein:

(a) the heavy chain comprises the amino acid
sequence:
(SEQ ID NO: 11)
QVQLVESGGGVVQPGRSLRLDCKASGITFSNSGMHWVRQAPGKGLEWVA
VIWYDGSKRYYADSVKGRFTISRDNSKNTLFLQMNSLRAEDTAVYYCAT
NDDYWGQGTLVTVSSASTKGPSVFPLAPCSRSTSESTAALGCLVKDYFP
EPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLG,
and
(b) the light chain comprises the amino acid
sequence:
(SEQ ID NO: 12)
EIVLTQSPATLSLSPGERATLSCRASQSVSSYLAWYQQKPGQAPRLLIY
DASNRATGIPARFSGSGSGTDFTLTISSLEPEDFAVYYCQQSSNWPRTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In some embodiments, the anti-PD-1 antibody comprises the six HVR sequences from SEQ ID NO:11 and SEQ ID NO:12 (e.g., the three heavy chain HVRs from SEQ ID NO:11 and the three light chain HVRs from SEQ ID NO:12). In some embodiments, the anti-PD-1 antibody comprises the heavy chain variable domain from SEQ ID NO:11 and the light chain variable domain from SEQ ID NO:12.

In some embodiments, the anti-PD-1 antibody is pembrolizumab (CAS Registry Number: 1374853-91-4). Pembrolizumab (Merck), also known as MK-3475, Merck 3475, lambrolizumab, KEYTRUDA®, and SCH-900475, is an anti-PD-1 antibody described in WO2009/114335. In some embodiments, the anti-PD-1 antibody comprises a heavy chain and a light chain sequence, wherein:

(a) the heavy chain comprises the amino acid
sequence:
(SEQ ID NO: 13)
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSSASTKGPSVFPLAPCSRSTSESTAALGC
LVKDYFPEPVTVSWNSGALTSGVHTFPAVLQSSGLYSLSSVVTVPSSSL
GTKTYTCNVDHKPSNTKVDKRVESKYGPPCPPCPAPEFLGGPSVFLFPP
KPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSL
SLSLG,
and
(b) the light chain comprises the amino acid
sequence:
(SEQ ID NO: 14)
EIVLTQSPATLSLSPGERATLSCRASKGVSTSGYSYLHWYQQKPGQAPR
LLIYLASYLESGVPARFSGSGSGTDFTLTISSLEPEDFAVYYCQHSRDL
PLTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC.

In some embodiments, the anti-PD-1 antibody comprises the six HVR sequences from SEQ ID NO:13 and SEQ ID NO:14 (e.g., the three heavy chain HVRs from SEQ ID NO:13 and the three light chain HVRs from SEQ ID NO:14). In some embodiments, the anti-PD-1 antibody comprises the heavy chain variable domain from SEQ ID NO:13 and the light chain variable domain from SEQ ID NO:14.

In some embodiments, the anti-PD-1 antibody is MEDI-0680 (AMP-514; AstraZeneca). MEDI-0680 is a humanized IgG4 anti-PD-1 antibody.

In some embodiments, the anti-PD-1 antibody is PDR001 (CAS Registry No. 1859072-53-9; Novartis). PDR001 is a humanized IgG4 anti-PD1 antibody that blocks the binding of PDL1 and PDL2 to PD-1.

In some embodiments, the anti-PD-1 antibody is REGN2810 (Regeneron). REGN2810 is a human anti-PD1 antibody also known as LIBTAYO® and cemiplimab-rwlc.

In some embodiments, the anti-PD-1 antibody is BGB-108 (BeiGene). In some embodiments, the anti-PD-1 antibody is BGB-A317 (BeiGene).

In some embodiments, the anti-PD-1 antibody is JS-001 (Shanghai Junshi). JS-001 is a humanized anti-PD1 antibody.

In some embodiments, the anti-PD-1 antibody is STI-A1110 (Sorrento). STI-A1110 is a human anti-PD1 antibody.

In some embodiments, the anti-PD-1 antibody is INCSHR-1210 (Incyte). INCSHR-1210 is a human IgG4 anti-PD1 antibody.

In some embodiments, the anti-PD-1 antibody is PF-06801591 (Pfizer).

In some embodiments, the anti-PD-1 antibody is TSR-042 (also known as ANB011; Tesaro/AnaptysBio).

In some embodiments, the anti-PD-1 antibody is AM0001 (ARMO Biosciences).

In some embodiments, the anti-PD-1 antibody is ENUM 244C8 (Enumeral Biomedical Holdings). ENUM 244C8 is an anti-PD1 antibody that inhibits PD-1 function without blocking binding of PDL1 to PD-1.

In some embodiments, the anti-PD-1 antibody is ENUM 388D4 (Enumeral Biomedical Holdings). ENUM 388D4 is an anti-PD1 antibody that competitively inhibits binding of PDL1 to PD-1.

In some embodiments, the PD-1 antibody comprises the six HVR sequences (e.g., the three heavy chain HVRs and the three light chain HVRs) and/or the heavy chain variable domain and light chain variable domain from a PD-1 antibody described in WO2015/112800 (Applicant: Regeneron), WO2015/112805 (Applicant: Regeneron), WO2015/112900 (Applicant: Novartis), US20150210769 (Assigned to Novartis), WO2016/089873 (Applicant: Celgene), WO2015/035606 (Applicant: Beigene), WO2015/085847 (Applicants: Shanghai Hengrui Pharmaceutical/Jiangsu Hengrui Medicine), WO2014/206107 (Applicants: Shanghai Junshi Biosciences/Junmeng Biosciences), WO2012/145493 (Applicant: Amplimmune), U.S. Pat. No. 9,205,148 (Assigned to MedImmune), WO2015/119930 (Applicants: Pfizer/Merck), WO2015/119923 (Applicants: Pfizer/Merck), WO2016/032927 (Applicants: Pfizer/Merck), WO2014/179664 (Applicant: AnaptysBio), WO2016/106160 (Applicant: Enumeral), and WO2014/194302 (Applicant: Sorrento).

In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. AMP-224 (CAS Registry No. 1422184-00-6; GlaxoSmithKline/MedImmune), also known as B7-DCIg, is a PDL2-Fc fusion soluble receptor described in WO2010/027827 and WO2011/066342.

In some embodiments, the PD-1 binding antagonist is a peptide or small molecule compound. In some embodiments, the PD-1 binding antagonist is AUNP-12 (PierreFabre/Aurigene). See, e.g., WO2012/168944, WO2015/036927, WO2015/044900, WO2015/033303, WO2013/144704, WO2013/132317, and WO2011/161699.

In some embodiments, the PDL1 binding antagonist is a small molecule that inhibits PD-1. In some embodiments, the PDL1 binding antagonist is a small molecule that inhibits PDL1. In some embodiments, the PDL1 binding antagonist is a small molecule that inhibits PDL1 and VISTA. In some embodiments, the PDL1 binding antagonist is CA-170 (also known as AUPM-170). In some embodiments, the PDL1 binding antagonist is a small molecule that inhibits PDL1 and TIM3. In some embodiments, the small molecule is a compound described in WO2015/033301 and WO2015/033299.

In some embodiments, the PD-1 axis binding antagonist is an anti-PDL1 antibody. A variety of anti-PDL1 antibodies are contemplated and described herein. In any of the embodiments herein, the isolated anti-PDL1 antibody can bind to a human PDL1, for example a human PDL1 as shown in UniProtKB/Swiss-Prot Accession No. Q9NZQ7.1, or a variant thereof. In some embodiments, the anti-PDL1 antibody is capable of inhibiting binding between PDL1 and PD-1 and/or between PDL1 and B7-1. In some embodiments, the anti-PDL1 antibody is a monoclonal antibody. In some embodiments, the anti-PDL1 antibody is an antibody fragment selected from the group consisting of Fab, Fab′-SH, Fv, scFv, and (Fab′)₂ fragments. In some embodiments, the anti-PDL1 antibody is a humanized antibody. In some embodiments, the anti-PDL1 antibody is a human antibody. Examples of anti-PDL1 antibodies useful for the methods of this invention, and methods for making thereof are described in PCT patent application WO 2010/077634 A1 and U.S. Pat. No. 8,217,149, which are incorporated herein by reference.

In some embodiments, the anti-PDL1 antibody comprises a heavy chain variable region and a light chain variable region, wherein:
(a) the heavy chain variable region comprises an HVR-H1, HVR-H2, and HVR-H3 sequence of GFTFSDSWIH (SEQ ID NO:1), AWISPYGGSTYYADSVKG (SEQ ID NO:2) and RHWPGGFDY (SEQ ID NO:3), respectively, and
(b) the light chain variable region comprises an HVR-L1, HVR-L2, and HVR-L3 sequence of RASQDVSTAVA (SEQ ID NO:4), SASFLYS (SEQ ID NO:5) and QQYLYHPAT (SEQ ID NO:6), respectively.

In some embodiments, the anti-PDL1 antibody is MPDL3280A, also known as atezolizumab and TECENTRIQ® (CAS Registry Number: 1422185-06-5), with a WHO Drug Information (International Nonproprietary Names for Pharmaceutical Substances), Proposed INN: List 112, Vol. 28, No. 4, published Jan. 16, 2015 (see page 485) described therein. In some embodiments, the anti-PDL1 antibody comprises a heavy chain and a light chain sequence, wherein:

(a) the heavy chain variable region sequence
comprises the amino acid sequence:
(SEQ ID NO: 7)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSS,
and
(b) the light chain variable region sequence
comprises the amino acid sequence:
(SEQ ID NO: 8)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR.

In some embodiments, the anti-PDL1 antibody comprises a heavy chain and a light chain sequence, wherein:

(a) the heavy chain comprises the amino acid
sequence:
(SEQ ID NO: 9)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYASTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPG,
and
(b) the light chain comprises the amino acid
sequence:
(SEQ ID NO: 10)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In some embodiments, the anti-PDL1 antibody is avelumab (CAS Registry Number: 1537032-82-8). Avelumab, also known as MSB0010718C, is a human monoclonal IgG1 anti-PDL1 antibody (Merck KGaA, Pfizer). In some embodiments, the anti-PDL1 antibody comprises a heavy chain and a light chain sequence, wherein:

(a) the heavy chain comprises the amino acid
sequence:
(SEQ ID NO: 15)
EVQLLESGGGLVQPGGSLRLSCAASGFTFSSYIMMWVRQAPGKGLEWVS
SIYPSGGITFYADTVKGRFTISRDNSKNTLYLQMNSLRAEDTAVYYCAR
IKLGTVTTVDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPG,
and
(b) the light chain comprises the amino acid
sequence:
(SEQ ID NO: 16)
QSALTQPASVSGSPGQSITISCTGTSSDVGGYNYVSWYQQHPGKAPKLM
IYDVSNRPSGVSNRFSGSKSGNTASLTISGLQAEDEADYYCSSYTSSST
RVFGTGTKVTVLGQPKANPTVTLFPPSSEELQANKATLVCLISDFYPGA
VTVAWKADGSPVKAGVETTKPSKQSNNKYAASSYLSLTPEQWKSHRSYS
CQVTHEGSTVEKTVAPTECS.

In some embodiments, the anti-PDL1 antibody comprises the six HVR sequences from SEQ ID NO:15 and SEQ ID NO:16 (e.g., the three heavy chain HVRs from SEQ ID NO:15 and the three light chain HVRs from SEQ ID NO:16). In some embodiments, the anti-PDL1 antibody comprises the heavy chain variable domain from SEQ ID NO:15 and the light chain variable domain from SEQ ID NO:16.

In some embodiments, the anti-PDL1 antibody is durvalumab (CAS Registry Number: 1428935-60-7). Durvalumab, also known as MEDI4736, is an Fc optimized human monoclonal IgG1 kappa anti-PDL1 antibody (MedImmune, AstraZeneca) described in WO2011/066389 and US2013/034559. In some embodiments, the anti-PDL1 antibody comprises a heavy chain and a light chain sequence, wherein:

(a) the heavy chain comprises the amino acid
sequence:
(SEQ ID NO: 17)
EVQLVESGGGLVQPGGSLRLSCAASGFTFSRYWMSWVRQAPGKGLEWVA
NIKQDGSEKYYVDSVKGRFTISRDNAKNSLYLQMNSLRAEDTAVYYCAR
EGGWFGELAFDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKRVEPKSCDKTHTCPPCPAPEFEGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPASIEKTISKA
KGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYT
QKSLSLSPG,
and
(b) the light chain comprises the amino acid
sequence:
(SEQ ID NO: 18)
EIVLTQSPGTLSLSPGERATLSCRASQRVSSSYLAWYQQKPGQAPRLLI
YDASSRATGIPDRFSGSGSGTDFTLTISRLEPEDFAVYYCQQYGSLPWT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC.

In some embodiments, the anti-PDL1 antibody comprises the six HVR sequences from SEQ ID NO:17 and SEQ ID NO:18 (e.g., the three heavy chain HVRs from SEQ ID NO:17 and the three light chain HVRs from SEQ ID NO:18). In some embodiments, the anti-PDL1 antibody comprises the heavy chain variable domain from SEQ ID NO:17 and the light chain variable domain from SEQ ID NO:18.

In some embodiments, the anti-PDL1 antibody is MDX-1105 (Bristol Myers Squibb). MDX-1105, also known as BMS-936559, is an anti-PDL1 antibody described in WO2007/005874.

In some embodiments, the anti-PDL1 antibody is LY3300054 (Eli Lilly).

In some embodiments, the anti-PDL1 antibody is STI-A1014 (Sorrento). STI-A1014 is a human anti-PDL1 antibody.

In some embodiments, the anti-PDL1 antibody is KN035 (Suzhou Alphamab). KN035 is single-domain antibody (dAB) generated from a camel phage display library.

In some embodiments, the anti-PDL1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some embodiments, the anti-PDL1 antibody is CX-072 (CytomX Therapeutics).

In some embodiments, the PDL1 antibody comprises the six HVR sequences (e.g., the three heavy chain HVRs and the three light chain HVRs) and/or the heavy chain variable domain and light chain variable domain from a PDL1 antibody described in US20160108123 (Assigned to Novartis), WO2016/000619 (Applicant: Beigene), WO2012/145493 (Applicant: Amplimmune), U.S. Pat. No. 9,205,148 (Assigned to MedImmune), WO2013/181634 (Applicant: Sorrento), and WO2016/061142 (Applicant: Novartis).

In a still further specific aspect, the antibody further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A.

In a still further specific aspect, the antibody has reduced or minimal effector function. In a still further specific aspect the minimal effector function results from an “effector-less Fc mutation” or aglycosylation mutation. In still a further embodiment, the effector-less Fc mutation is an N297A or D265A/N297A substitution in the constant region. In some embodiments, the isolated anti-PDL1 antibody is aglycosylated. Glycosylation of antibodies is typically either N-linked or O-linked N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

In a still further embodiment, the present disclosure provides for compositions comprising any of the above described anti-PDL1 antibodies in combination with at least one pharmaceutically-acceptable carrier.

In a still further embodiment, the present disclosure provides for a composition comprising an anti-PDL1, an anti-PD-1, or an anti-PDL2 antibody or antigen binding fragment thereof as provided herein and at least one pharmaceutically acceptable carrier. In some embodiments, the anti-PDL1, anti-PD-1, or anti-PDL2 antibody or antigen binding fragment thereof administered to the individual is a composition comprising one or more pharmaceutically acceptable carrier. Any of the pharmaceutically acceptable carriers described herein or known in the art may be used.

### V. Antibody Preparation

The antibody described herein is prepared using techniques available in the art for generating antibodies, exemplary methods of which are described in more detail in the following sections.

The antibody is directed against an antigen of interest (e.g., PD-1 or PD-L1, such as a human PD-1 or PD-L1). Preferably, the antigen is a biologically important polypeptide and administration of the antibody to a mammal suffering from a disorder can result in a therapeutic benefit in that mammal.

In certain embodiments, an antibody provided herein has a dissociation constant (Kd) of ≤1 μM, ≤150 nM, ≤100 nM, ≤50 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10 M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [125I]-antigen are mixed with serial dilutions of a Fab of interest. The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CMS chips at ˜10 response units (RU). Briefly, carboxymethylated dextran biosensor chips (CMS, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 106 M-1 s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

Chimeric, Humanized and Human Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB™ technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Antibody Fragments

Antibody fragments may be generated by traditional means, such as enzymatic digestion, or by recombinant techniques. In certain circumstances there are advantages of using antibody fragments, rather than whole antibodies. The smaller size of the fragments allows for rapid clearance, and may lead to improved access to solid tumors. For a review of certain antibody fragments, see Hudson et al. (2003) Nat. Med. 9:129-134.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′) 2 fragments can be isolated directly from recombinant host cell culture. Fab and F(ab′) 2 fragment with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See WO 93/16185; U.S. Pat. Nos. 5,571,894; and 5,587,458. Fv and scFv are the only species with intact combining sites that are devoid of constant regions; thus, they may be suitable for reduced nonspecific binding during in vivo use. scFv fusion proteins may be constructed to yield fusion of an effector protein at either the amino or the carboxy terminus of an scFv. See Antibody Engineering, ed. Borrebaeck, supra. The antibody fragment may also be a “linear antibody”, e.g., as described in U.S. Pat. No. 5,641,870, for example. Such linear antibodies may be monospecific or bispecific.

Single-Domain Antibodies

In some embodiments, an antibody of the present disclosure is a single-domain antibody. A single-domain antibody is a single polypeptide chain comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1). In one embodiment, a single-domain antibody consists of all or a portion of the heavy chain variable domain of an antibody.

Antibody Variants

In some embodiments, amino acid sequence modification(s) of the antibodies described herein are contemplated. For example, it may be desirable to improve the binding affinity and/or other biological properties of the antibody Amino acid sequence variants of the antibody may be prepared by introducing appropriate changes into the nucleotide sequence encoding the antibody, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics. The amino acid alterations may be introduced in the subject antibody amino acid sequence at the time that sequence is made.

Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 2. More substantial changes are described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2
Conservative Substitutions.

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
a. hydrophobic: Norleucine, Met, Ala, Val, Leu, Ile;
b. neutral hydrophilic: Cys, Ser, Thr, Asn, Gln;
c. acidic: Asp, Glu;
d. basic: His, Lys, Arg;
e. residues that influence chain orientation: Gly, Pro;
f. aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the present disclosure may be made in order to create antibody variants with certain improved properties.

In one embodiment, antibody variants are provided comprising an Fc region wherein a carbohydrate structure attached to the Fc region has reduced fucose or lacks fucose, which may improve ADCC function. Specifically, antibodies are contemplated herein that have reduced fucose relative to the amount of fucose on the same antibody produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In certain embodiments, the antibody is one wherein less than about 50%, 40%, 30%, 20%, 10%, or 5% of the N-linked glycans thereon comprise fucose. For example, the amount of fucose in such an antibody may be from 1% to 80%, from 1% to 65%, from 5% to 65% or from 20% to 40%. In certain embodiments, the antibody is one wherein none of the N-linked glycans thereon comprise fucose, i.e., wherein the antibody is completely without fucose, or has no fucose or is afucosylated. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93(5): 851-861 (2006). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In certain embodiments, the antibody variants comprising an Fc region described herein are capable of binding to an FcγRIII. In certain embodiments, the antibody variants comprising an Fc region described herein have ADCC activity in the presence of human effector cells or have increased ADCC activity in the presence of human effector cells compared to the otherwise same antibody comprising a human wild-type IgG1Fc region.

Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the present disclosure contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues). In an exemplary embodiment, the antibody comprising the following amino acid substitutions in its Fc region: S298A, E333A, and K334A.

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.)). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

### VI. Pharmaceutical Compositions and Formulations

Also provided herein are pharmaceutical compositions and formulations, e.g., for the treatment of cancer, or for inducing neoepitope-specific immune responses according to the methods described herein. In some embodiments, the pharmaceutical compositions and formulations further comprise a pharmaceutically acceptable carrier.

After preparation of the antibody of interest (e.g., techniques for producing antibodies which can be formulated as disclosed herein are elaborated herein and are known in the art), the pharmaceutical formulation comprising it is prepared. In certain embodiments, the antibody to be formulated has not been subjected to prior lyophilization and the formulation of interest herein is an aqueous formulation. In certain embodiments, the antibody is a full length antibody. In one embodiment, the antibody in the formulation is an antibody fragment, such as an F(ab′)₂, in which case problems that may not occur for the full length antibody (such as clipping of the antibody to Fab) may need to be addressed. The therapeutically effective amount of antibody present in the formulation is determined by taking into account the desired dose volumes and mode(s) of administration, for example. From about 25 mg/mL to about 150 mg/mL, or from about 30 mg/mL to about 140 mg/mL, or from about 35 mg/mL to about 130 mg/mL, or from about 40 mg/mL to about 120 mg/mL, or from about 50 mg/mL to about 130 mg/mL, or from about 50 mg/mL to about 125 mg/mL, or from about 50 mg/mL to about 120 mg/mL, or from about 50 mg/mL to about 110 mg/mL, or from about 50 mg/mL to about 100 mg/mL, or from about 50 mg/mL to about 90 mg/mL, or from about 50 mg/mL to about 80 mg/mL, or from about 54 mg/mL to about 66 mg/mL is an exemplary antibody concentration in the formulation. In some embodiments, an anti-PDL1 antibody described herein (such as atezolizumab) is administered at a dose of about 1200 mg. In some embodiments, an anti-PD1 antibody described herein (such as pembrolizumab) is administered at a dose of about 200 mg. In some embodiments, an anti-PD1 antibody described herein (such as nivolumab) is administered at a dose of about 240 mg (e.g., every 2 weeks) or 480 mg (e.g., every 4 weeks).

In some embodiments, an RNA vaccine described herein is administered at a dose of about 15 μg, about 25 μg, about 38 μg, about 50 μg, or about 100 μg.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The composition and formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. The formulations to be used for in vivo administration are generally sterile. Sterility may be readily accomplished, e.g., by filtration through sterile filtration membranes.

Pharmaceutical formulations of atezolizumab and pembrolizumab are commercially available. For example, atezolizumab is known under the trade name (as described elsewhere herein) TECENTRIQ®. Pembrolizumab is known under the trade name (as described elsewhere herein) KEYTRUDA®. In some embodiments, atezolizumab and the RNA vaccine, or pembrolizumab and the RNA vaccine, are provided in separate containers. In some embodiments, atezolizumab and pembrolizumab are used and/or prepared for administration to an individual as described in the prescribing information available with the commercially available product.

### VII. Methods of Treatment

Provided herein are methods for treating or delaying progression of cancer in an individual (e.g., by inducing a neoepitope-specific immune response according to the methods provided herein), comprising administering to the individual an effective amount of an RNA vaccine as a single agent or in combination with a PD-1 axis binding antagonist. In some embodiments, the individual is human.

Any of the PD-1 axis binding antagonists and RNA vaccines of the present disclosure may find use in the methods of treatment described herein. In some embodiments, the RNA vaccine comprises one or more polynucleotides encoding 10-20 neoepitopes resulting from cancer-specific somatic mutations present in the tumor specimen. In some embodiments, the RNA vaccine comprises one or more polynucleotides encoding 5-20 neoepitopes resulting from cancer-specific somatic mutations present in the tumor specimen. In some embodiments, the RNA vaccine is formulated in a lipoplex nanoparticle or liposome. In some embodiments, a lipoplex nanoparticle formulation for the RNA (RNA-Lipoplex) is used to enable IV delivery of an RNA vaccine of the present disclosure. In some embodiments, the PCV is administered intravenously, for example, in a liposomal formulation, at doses of 15 μg, 25 μg, 38 μg, 50 μg, or 100 μg. In some embodiments, 15 μg, 25 μg, 38 μg, 50 μg, or 100 μg of RNA is delivered per dose (i.e., dose weight reflects the weight of RNA administered, not the total weight of the formulation or lipoplex administered). More than one PCV may be administered to a subject, e.g., subject is administered one PCV with a combination of neoepitopes and also administered a separate PCV with a different combination of neoepitopes. In some embodiments, a first PCV with ten neoepitopes is administered in combination with a second PCV with ten alternative epitopes. In some embodiments, the PD-1 axis binding antagonist is an anti-PD-1 antibody, including without limitation pembrolizumab. In some embodiments, the PD-1 axis binding antagonist is an anti-PD-L1 antibody, including without limitation atezolizumab.

In some embodiments, the PD-1 axis binding antagonist is administered to the individual at an interval of 21 days or 3 weeks. In some embodiments, the PD-1 axis binding antagonist is an anti-PD-1 antibody (e.g., pembrolizumab) administered to the individual at an interval of 21 days or 3 weeks, e.g., at a dose of about 200 mg. In some embodiments, the PD-1 axis binding antagonist is an anti-PD-1 antibody (e.g., cemiplimab-rwlc) administered to the individual at an interval of 21 days or 3 weeks, e.g., at a dose of about 350 mg. In some embodiments, the PD-1 axis binding antagonist is an anti-PD-L1 antibody (e.g., atezolizumab) administered to the individual at an interval of 21 days or 3 weeks, e.g., at a dose of about 1200 mg.

In some embodiments, the PD-1 axis binding antagonist is administered to the individual at an interval of 14 days or 28 days. In some embodiments, the PD-1 axis binding antagonist is administered to the individual at an interval of 2 weeks or 4 weeks. In some embodiments, the PD-1 axis binding antagonist is an anti-PD-1 antibody (e.g., nivolumab) administered to the individual at an interval of 14 days, 2 weeks, 28 days, or 4 weeks, e.g., at a dose of about 240 mg at an interval of 14 days or 2 weeks, or at a dose of about 480 mg at an interval of 28 days or 4 weeks. In some embodiments, the PD-1 axis binding antagonist is an anti-PD-1 antibody (e.g., nivolumab) administered to the individual at an interval of 21 days or 3 weeks, e.g., at a dose of about lmg/kg for 1, 2, 3, or 4 doses, optionally in combination with an anti-CTLA-4 antibody (e.g., ipilimumab), and optionally followed by administration of the anti-PD-1 antibody (e.g., nivolumab) alone at an interval of 14 days, 2 weeks, 28 days, or 4 weeks, e.g., at a dose of about 240 mg at an interval of 14 days or 2 weeks, or at a dose of about 480 mg at an interval of 28 days or 4 weeks.

In some embodiments, the PD-1 axis binding antagonist is administered to the individual at an interval of 14 days or 2 weeks. In some embodiments, the PD-1 axis binding antagonist is an anti-PD-L1 antibody (e.g., durvalumab) administered to the individual at an interval of 14 days or 2 weeks, e.g., at a dose of about 10 mg/kg (optionally by intravenous infusion over 60 minutes). In some embodiments, the PD-1 axis binding antagonist is an anti-PD-L1 antibody (e.g., avelumab) administered to the individual at an interval of 14 days or 2 weeks, e.g., at a dose of about 10 mg/kg (optionally by intravenous infusion over 60 minutes).

In some embodiments, the RNA vaccine is administered to the individual at an interval of 21 days or 3 weeks.

In some embodiments, the PD-1 axis binding antagonist and the RNA vaccine are administered to the individual in 8 21-day Cycles. In some embodiments, the RNA vaccine is administered to the individual on Days 1, 8, and 15 of Cycle 2 and Day 1 of Cycles 3-7. In some embodiments, the PD-1 axis binding antagonist is administered to the individual on Day 1 of Cycles 1-8. In some embodiments, the RNA vaccine is administered to the individual on Days 1, 8, and 15 of Cycle 2 and Day 1 of Cycles 3-7, and the PD-1 axis binding antagonist is administered to the individual on Day 1 of Cycles 1-8.

In some embodiments, the PD-1 axis binding antagonist and the RNA vaccine are further administered to the individual after Cycle 8. In some embodiments, the PD-1 axis binding antagonist and the RNA vaccine are further administered to the individual in 17 additional 21-day Cycles, wherein the PD-1 axis binding antagonist is administered to the individual on Day 1 of Cycles 13-29, and/or wherein the RNA vaccine is administered to the individual on Day 1 of Cycles 13, 21, and 29.

In certain embodiments, a PD-1 axis binding antagonist and an RNA vaccine are administered to the individual in 8 21-day Cycles, wherein the PD-1 axis binding antagonist is pembrolizumab and is administered to the individual at a dose of about 200 mg on Day 1 of Cycles 1-8, and wherein the RNA vaccine is administered to the individual at a dose of about 25 μg on Days 1, 8, and 15 of Cycle 2 and Day 1 of Cycles 3-7. In certain embodiments, a PD-L1 axis binding antagonist and the RNA vaccine are administered to the individual in 8 21-day Cycles, wherein the PD-L1 axis binding antagonist is atezolizumab and is administered to the individual at a dose of about 1200 mg on Day 1 of Cycles 1-8, and wherein the RNA vaccine is administered to the individual at a dose of about 25 μg on Days 1, 8, and 15 of Cycle 2 and Day 1 of Cycles 3-7. In some embodiments, the RNA vaccine is administered to the individual at doses of about 25 μg on Day 1 of Cycle 2, about 25 μg on Day 8 of Cycle 2, about 25 μg on Day 15 of Cycle 2, and about 25 μg on Day 1 of each of Cycles 3-7 (that is to say, a total of about 75 μg of the vaccine is administered to the individual over 3 doses during Cycle 2). In some embodiments, a total of about 75 μg of the vaccine is administered to the individual over 3 doses during the first Cycle in which the RNA vaccine is administered.

In certain embodiments, a PD-1 axis binding antagonist and an RNA vaccine are administered to the individual in 8 21-day Cycles, wherein the PD-1 axis binding antagonist is pembrolizumab and is administered to the individual at a dose of 200 mg on Day 1 of Cycles 1-8, and wherein the RNA vaccine is administered to the individual at a dose of 25 μg on Days 1, 8, and 15 of Cycle 2 and Day 1 of Cycles 3-7. In certain embodiments, a PD-L1 axis binding antagonist and the RNA vaccine are administered to the individual in 8 21-day Cycles, wherein the PD-L1 axis binding antagonist is atezolizumab and is administered to the individual at a dose of 1200 mg on Day 1 of Cycles 1-8, and wherein the RNA vaccine is administered to the individual at a dose of 25 μg on Days 1, 8, and 15 of Cycle 2 and Day 1 of Cycles 3-7. In some embodiments, the RNA vaccine is administered to the individual at doses of 25 μg on Day 1 of Cycle 2, 25 μg on Day 8 of Cycle 2, 25 ng on Day 15 of Cycle 2, and 25 μg on Day 1 of each of Cycles 3-7 (that is to say, a total of 75 μg of the vaccine is administered to the individual over 3 doses during Cycle 2). In some embodiments, a total of 75 μg of the vaccine is administered to the individual over 3 doses during the first Cycle in which the RNA vaccine is administered.

In some embodiments, the RNA vaccine is administered to the individual at a dose of between about 15 μg to about 100 μg (e.g., any of about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, or about 100 μg). In some embodiments, the RNA vaccine is administered to the individual at a dose of about 15 μg, about 25 μg, about 38 μg, about 50 μg, about 75 μg, or about 100 μg. In certain embodiments, the RNA vaccine is administered intravenously to the individual.

In some embodiments, the RNA vaccine is administered to the individual at an interval of 7 days or 1 week. In certain embodiments, the RNA vaccine is administered to the individual at an interval of 14 days or 2 weeks. In certain embodiments, the RNA vaccine is administered to the individual for 12 weeks.

In some embodiments, the RNA vaccine is administered to the individual in four 21-day Cycles, wherein the RNA vaccine is administered to the individual on Days 1, 8, and 15 of Cycle 1; Days 1, 8, and 15 of Cycle 2; Days 1 and 15 of Cycle 3; and Day 1 of Cycle 4.

In some embodiments, the RNA vaccine is administered to the individual in an induction stage and a maintenance stage after the induction stage, wherein the RNA vaccine is administered to the individual during the induction stage at an interval of 1 or 2 weeks, and wherein the RNA vaccine is administered to the individual during the maintenance stage at an interval of 24 weeks. In certain embodiments, the RNA vaccine is administered to the individual in an induction stage and a maintenance stage after the induction stage, wherein the RNA vaccine is administered to the individual during the induction stage at an interval of 7 days or 14 days, and wherein the RNA vaccine is administered to the individual during the maintenance stage at an interval of 168 days.

In some embodiments, the RNA vaccine is administered to the individual in an induction stage and a maintenance stage after the induction stage, wherein the RNA vaccine is administered to the individual during the induction stage in four 21-day Cycles, wherein the RNA vaccine is administered to the individual during the induction stage on Days 1, 8, and 15 of Cycle 1; Days 1, 8, and 15 of Cycle 2; Days 1 and 15 of Cycle 3; and Day 1 of Cycle 4; and wherein the RNA vaccine is administered to the individual during the maintenance stage on Day 1 of Cycle 5 and once every 24 weeks or 168 days thereafter.

The PD-1 axis binding antagonist and the RNA vaccine may be administered in any order. For example, a PD-1 axis binding antagonist and an RNA vaccine may be administered sequentially (at different times) or concurrently (at the same time). In some embodiments, a PD-1 axis binding antagonist and an RNA vaccine are in separate compositions. In some embodiments, a PD-1 axis binding antagonist and an RNA vaccine are in the same composition.

In some embodiments, the cancer is selected from the group consisting of melanoma, non-small cell lung cancer, bladder cancer, colorectal cancer, triple negative breast cancer, renal cancer, and head and neck cancer. In some embodiments, the cancer is locally advanced or metastatic melanoma, non-small cell lung cancer, bladder cancer, colorectal cancer, triple negative breast cancer, renal cancer, or head and neck cancer. In some embodiments, the cancer is selected from the group consisting of non-small cell lung cancer, bladder cancer, colorectal cancer, triple negative breast cancer, renal cancer, and head and neck cancer. In some embodiments, the cancer is locally advanced or metastatic non-small cell lung cancer, bladder cancer, colorectal cancer, triple negative breast cancer, renal cancer, or head and neck cancer.

In some embodiments, the cancer is melanoma. In some embodiments, the melanoma is cutaneous or mucosal melanoma. In some embodiments, the melanoma is cutaneous, mucosal, or acral melanoma. In some embodiments, the melanoma is not ocular or acral melanoma. In some embodiments, the melanoma is metastatic or unresectable locally advanced melanoma. In some embodiments, the melanoma is stage IV melanoma. In some embodiments, the melanoma is stage IIIC or stage IIID melanoma. In some embodiments, the melanoma is unresectable or metastatic melanoma. In some embodiments, the method provides adjuvant treatment of melanoma.

In some embodiments, the cancer (e.g., melanoma) is previously untreated. In some embodiments, the cancer is previously untreated advanced melanoma.

In some embodiments, the tumor is a non-small cell lung (NSCLC), bladder, renal, head and neck, sarcoma, breast, melanoma, prostate, ovarian, gastric, liver, or colorectal tumor. In some embodiments, wherein the breast tumor is a triple-negative breast (TNBC) tumor. In some embodiments, prior to administration of the RNA vaccine, the individual has been treated with one or more cancer therapies. In some embodiments, prior to administration of the RNA vaccine, the individual has been treated with a checkpoint inhibitor therapy. In some embodiments, prior to administration of the RNA vaccine, the individual has not been treated with a checkpoint inhibitor therapy.

In some embodiments, prior to treatment with a PD-1 axis binding antagonist and an RNA vaccine according to any of the methods described herein, the individual has progressed after treatment with or failed to respond adequately to treatment with a PD-1 axis binding antagonist-based monotherapy, e.g., treatment with pembrolizumab in the absence of an RNA vaccine.

The PD-1 axis binding antagonist and the RNA vaccine may be administered by the same route of administration or by different routes of administration. In some embodiments, the PD-1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some embodiments, the RNA vaccine is administered (e.g., in a lipoplex particle or liposome) intravenously, intramuscularly, subcutaneously, topically, orally, transdermally, intraperitoneally, intraorbitally, by implantation, by inhalation, intrathecally, intraventricularly, or intranasally. In some embodiments, the PD-1 axis binding antagonist and the RNA vaccine are administered via intravenous infusion. An effective amount of the PD-1 axis binding antagonist and the RNA vaccine may be administered for prevention or treatment of disease.

In some embodiments, the methods may further comprise an additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation.

### VIII. Articles of Manufacture or Kits

Further provided herein is an article of manufacture or kit comprising an RNA vaccine of the present disclosure. Further provided herein is an article of manufacture or a kit comprising a PD-1 axis binding antagonist (such as atezolizumab or pembrolizumab). In some embodiments, the article of manufacture or kit further comprises package insert comprising instructions for using the RNA vaccine and/or PD-1 axis binding antagonist (e.g., in conjunction with the RNA vaccine) to treat or delay progression of cancer in an individual, enhance immune function of an individual having cancer, induce neoepitope-specific T cells in an individual with a tumor, and/or induce trafficking of neoepitope-specific T cells in an individual to a tumor. Also provided herein is an article of manufacture or a kit comprising a PD-1 axis binding antagonist (such as atezolizumab or pembrolizumab) and an RNA vaccine.

In some embodiments, the PD-1 axis binding antagonist and the RNA vaccine are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The present disclosure will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: A Study of an RNA Vaccine as a Single Agent and in Combination with Atezolizumab in Patients with Locally Advanced or Metastatic Tumors

This Example describes a Phase 1a/1b, open-label, multicenter, global, dose-escalation study designed to evaluate the safety, tolerability, immune response, and pharmacokinetics of a neoantigen specific RNA vaccine as a single agent and in combination with the anti-PD-L1 antibody atezolizumab.

### Study Objectives

The objectives of this study are to evaluate the safety, tolerability, immune response, and pharmacokinetics of the RNA vaccine as a single agent and in combination with atezolizumab.

### Study Design

Phase 1a

In the Phase 1a dose escalation cohort of this study, patients are administered the RNA vaccine by intravenous (IV) infusion at escalating doses in 21-day cycles.

Phase 1b

The Phase 1b of this study includes a dose escalation cohort, an exploration cohort, an expansion cohort, and an expansion cohort with serial biopsies.

In the Phase 1b dose escalation cohort of this study, patients are administered the RNA vaccine by IV infusion at escalating doses in 21-day cycles. Patients are also administered a fixed dose of 1200 mg atezolizumab on day 1 of every 21-day cycle.

In the Phase 1b exploration cohort of this study, patients with non-small cell lung cancer (NSCLC) or melanoma that have been previously treated with cancer immunotherapy (CIT) are administered the RNA vaccine at a dose lower than the Maximum Tolerated Dose (MTD) by IV infusion in 21-day cycles. Patients are also administered a fixed dose of 1200 mg atezolizumab on day 1 of every 21-day cycle.

In the Phase 1b expansion cohort of this study, patients with the indications described in the study Inclusion Criteria below are administered the RNA vaccine at multiple dose levels lower than the MTD by IV infusion in 21-day cycles. Patients are also administered a fixed dose of 1200 mg atezolizumab on day 1 of every 21-day cycle.

In the Phase 1b expansion cohort with serial biopsies of this study, CIT-naïve patients with the tumor types described in the study Inclusion Criteria below are administered the RNA vaccine at multiple dose levels lower than the MTD by IV infusion in 21-day cycles. Patients are also administered a fixed dose of 1200 mg atezolizumab on day 1 of every 21-day cycle.

### Study Participants

Inclusion Criteria

Patients that meet the following criteria are included in this study:
Eastern Cooperative Oncology Group (ECOG) performance status of 0 or 1.
Histologic documentation of locally advanced, recurrent, or metastatic incurable malignancy that has progressed after at least one available standard therapy; or for whom standard therapy has proven to be ineffective or intolerable, or is considered inappropriate.
Measurable disease according the Response Evaluation Criteria for Solid Tumors Version 1.1 (RECIST v1.1).

In addition, patients that meet the following indication-specific criteria are included in the exploration or expansion cohorts of Phase 1b of this study:
Non-small cell lung cancer (NSCLC) cohort (CIT-naïve): Patients with histologically confirmed incurable, advanced NSCLC not previously treated with anti-PD-L1/PD-1 and/or anti-CTLA-4 therapies.
NSCLC cohort (CIT-treated): Patients with histologically confirmed incurable, advanced NSCLC previously treated with an anti-PD-L1/PD-1 therapy with or without an anti-CTLA-4 therapy.
Triple negative breast cancer (TNBC) cohort (CIT-naïve): Patients with histologically confirmed incurable, advanced estrogen receptor (ER)-negative, progesterone receptor-negative, and human epidermal growth factor receptor 2 (HER2)-negative adenocarcinoma of the breast (triple-negative) not previously treated with anti-PD-L1/PD-1 and/or anti-CTLA-4 therapies.
Colorectal cancer (CRC) cohort (CIT-naïve): Patients with histologically confirmed incurable, advanced adenocarcinoma of the colon or rectum not previously treated with anti-PD-L1/PD-1 and/or anti-CTLA-4 therapies.
Head and neck squamous cell carcinoma (HNSCC) cohort (CIT-naïve): Patients with histologically confirmed inoperable, locally advanced or metastatic, recurrent, or persistent HNSCC (oral cavity, oropharynx, hypopharnyx, or larynx) not amenable to curative therapy and not previously treated with anti-PDL1/PD-1 and/or anti-CTLA-4 therapies.
Urothelial carcinoma (UC) cohort (CIT-naïve): Patients with histologically confirmed incurable, advanced transitional cell carcinoma of the urothelium including renal pelvis, ureters, urinary bladder, and urethra, not previously treated with an anti-PD-L1/PD-1 therapy with or without an anti-CTLA-4 therapy.
UC cohort (CIT-treated): Patients with histologically confirmed incurable advanced transitional cell carcinoma of the urothelium (including renal pelvis, ureters, urinary bladder, and urethra) previously treated with an anti-PD-L1/PD-1 therapy with or without an anti-CTLA-4 therapy.
Renal cell carcinoma (RCC) cohort (CIT-naïve): Patients with histologically confirmed incurable, advanced RCC with component of clear cell histology and/or component of sarcomatoid histology not previously treated with anti-PD-L1/PD-1 and/or anti-CTLA-4 therapies.
Melanoma cohort (CIT-naïve in metastatic setting): Patients with histologically confirmed incurable, advanced melanoma not previously treated with anti-PD-L1/PD-1 and/or anti-CTLA-4 therapies in the metastatic setting.
Melanoma cohort (CIT-treated): Patients with histologically confirmed incurable, advanced melanoma previously treated with anti-PD-L1/PD-1 and/or anti-CTLA-4 therapies.

In addition, patients that meet the following indication-specific criteria are included in the serial-biopsy expansion cohort of Phase 1b of this study:
Patients have one of the locally advanced or metastatic solid tumor types specified in the study Inclusion Criteria above.
Patients have accessible lesions that permit a total of two to three biopsies (pre-treatment and on-treatment) or one biopsy (on-treatment, if archival tissue is available in place of a pre-treatment biopsy) without unacceptable risk of a significant procedural complication. RECIST lesions are not biopsied.

Exclusion Criteria

Patients that meet the following criteria are excluded from this study:
Clinically significant liver disease.
Previous splenectomy.
Primary immunodeficiencies, either cellular (e.g., DiGeorge syndrome, T-negative severe combined immunodeficiency [SCID]) or combined T- and B-cell immunodeficiencies (e.g., T- and B-negative SCID, Wiskott Aldrich syndrome, ataxia telangiectasia, common variable immunodeficiency).
Any anti-cancer therapy, including chemotherapy, hormonal therapy, and/or radiotherapy, within 3 weeks prior to initiation of study treatment, unless otherwise specified.
Prior neoantigen-specific or whole-tumor cancer vaccine, unless otherwise specified.
Prior treatment with cytokines is allowed provided that at least 6 weeks or 5 half-lives of the drug, whichever is shorter, have elapsed between the last dose and day 1 of cycle 1 of this study.
Prior treatment with immune checkpoint inhibitors, immunomodulatory monoclonal antibody (mAb), and/or mAb-derived therapies is allowed provided that at least 6 weeks (Phase 1a) or 3 weeks (Phase 1b) have elapsed between the last dose and day 1 of cycle 1 of this study, unless otherwise specified.
In the CIT-naïve expansion cohort in Phase Ib, prior treatment with an anti-PD-L1/PD-1 therapy and/or an anti-CTLA-4 therapy is not allowed.
In the melanoma CIT-naïve expansion cohort in Phase Ib, prior treatment with an anti-PD-L1/PD-1 therapy and/or an anti-CTLA-4 therapy in the metastatic setting is not allowed.
Prior treatment with immunomodulators, including toll-like receptor (TLR) agonists, inhibitors of indoleamine 2,3-dioxygenase (IDO)/tryptophan-2,3-dioxygenase (TDO), or agonists of OX40 is allowed provided that at least 5 half-lives of the drug or a minimum of 3 weeks have elapsed between the last dose of the prior treatment and day 1 of cycle 1 of this study, unless otherwise specified.
Any history of an immune-related Grade 4 adverse event attributed to prior CIT (other than endocrinopathy managed with replacement therapy or asymptomatic elevation of serum amylase or lipase).
Any history of an immune-related Grade 3 adverse event attributed to prior CIT (other than hypothyroidism managed with replacement therapy) that resulted in permanent discontinuation of the prior immunotherapeutic agent and/or occurred less than or equal to 6 months prior to day 1 of cycle 1 of this study.
Adverse events from prior anti-cancer therapy that have not resolved to less than or equal to Grade 1 except for alopecia, vitiligo, or endocrinopathy managed with replacement therapy.
All immune-related adverse events related to prior CIT (other than endocrinopathy managed with replacement therapy or stable vitiligo) must have resolved completely to baseline.
Primary central nervous system (CNS) malignancy, untreated CNS metastases, or active CNS metastases (progressing or requiring corticosteroids for symptomatic control).
Malignancies other than disease under study within 5 years prior to day 1 of cycle 1 of this study, with the exception of those with a negligible risk of metastasis or death.
Leptomeningeal disease.
Spinal cord compression not definitively treated with surgery and/or radiation, or previously diagnosed and treated spinal cord compression without evidence that the disease has been clinically stable for greater than or equal to 2 weeks prior to screening.
Uncontrolled hypercalcemia, pleural effusion, pericardial effusion, or ascites requiring recurrent drainage procedures, or tumor-related pain.
History of autoimmune disease, unless otherwise specified.
Treatment with monoamine oxidase inhibitors (MAOIs) within 3 weeks prior to day 1 of cycle 1 of this study.
Treatment with systemic immunosuppressive medications within 2 weeks prior to day 1 of cycle 1 of this study.
History of idiopathic pulmonary fibrosis, pneumonitis, organizing pneumonia, or evidence of active pneumonitis on screening chest computed tomography (CT) scan; positive test for human immunodeficiency virus infection; active hepatitis B or C; active or latent tuberculosis infection; or severe infections within 4 weeks prior to day 1 of cycle 1 of this study.
Prior allogeneic bone marrow transplantation or prior solid organ transplantation.

### Study Outcome Measures

The primary outcome measures of this study include the following:
The percentage of patients with dose-limiting toxicities (DLTs), assessed from days 1-14 in Phase 1a of this study and from days 1-21 in Phase 1b of this study.
The maximum tolerated dose (MTD) and the recommended phase 2 dose (RP2D) for the RNA vaccine, assessed from days 1-14 in Phase 1a of this study and from days 1-21 in Phase 1b of this study.
The percentage of patients with adverse events (AEs), assessed from baseline until the end of the study. The severity of AEs is assessed according to the National Cancer Institute (NCI) Common Terminology Criteria for Adverse Events (CTCAE) Version 5.0.
The percentage of patients with immune-mediated adverse events (imAEs) (NCI CTCAE Version 5.0), assessed from baseline until the end of the study.
The number of treatment cycles received by patients, assessed from baseline until the end of the study.
   The dose intensity of RNA vaccine, assessed from baseline until the end of the study.
   Changes from baseline in vital signs, clinical laboratory test results and ECGs, assessed from baseline until the end of the study.

The secondary outcome measures of this study include the following:
Plasma concentration of (R)-N,N,N-Trimethyl-2,3-Dioleyloxy-1-Propanaminium Chloride (DOTMA), assessed from pre-infusion until treatment discontinuation.
Plasma concentration of ribonucleic acid (RNA), assessed from pre-infusion until treatment discontinuation.
Serum concentration of atezolizumab, assessed from pre-infusion until 2 months post treatment discontinuation.
The percentage of patients with induction of antigen-specific T-cell responses in peripheral blood, assessed from pre-infusion until treatment discontinuation.
The levels of immune-related cytokines, assessed from pre-infusion until treatment discontinuation.
The percentage of patients with objective response of complete response (CR) or partial response (PR) according to RECIST v1.1, assessed from baseline until 90 days after the last dose of study treatment or initiation of another systemic anti-cancer therapy, whichever occurs first.
Duration of response (DoR) according to RECIST v1.1, assessed from the first occurrence of a documented CR or PR until disease progression or death due to any cause, whichever occurs first.
The percentage of patients with objective response of CR or PR according to the Immune-Modified RECIST, assessed from baseline until 90 days after the last dose of study treatment or initiation of another systemic anticancer therapy, whichever occurs first.
DoR according to the Immune-Modified RECIST, assessed from the first occurrence of a documented CR or PR until disease progression or death due to any cause, whichever occurs first.
Progression-free survival (PFS) according to RECIST v1.1, assessed from baseline until 90 days after the last dose of study treatment or initiation of another systemic anti-cancer therapy, whichever occurs first.
Overall survival (OS), assessed from baseline until 90 days after the last dose of study treatment or initiation of another systemic anti-cancer therapy.
The percentage of patients with anti-drug antibodies (ADAs) to atezolizumab, assessed from pre-infusion to 2 months post treatment discontinuation.

### Example 2: Phase 1a/Lb Studies of an RNA Vaccine as a Single Agent and in Combination with Atezolizumab in Patients with Locally Advanced or Metastatic Solid Tumors

Neoantigens arising from somatic mutations are attractive targets for cancer immunotherapy as they may be recognized as foreign by the immune system. An RNA lipoplex vaccine was designed to stimulate T cell responses against neoantigens. As described in Example 1, a first-in-human Phase Ia study of the RNA vaccine was conducted in patients with locally advanced or metastatic solid tumors.

The RNA vaccine was manufactured on a per-patient basis and contained up to 20 tumor-specific neoepitopes. Nine doses of the RNA vaccine were systemically administered i.v. at weekly and bi-weekly intervals during the 12-cycle induction stage and every 24 weeks during the maintenance stage. Specifically, the RNA vaccine was administered in four 21-day Cycles during the induction stage: on Days 1, 8, and 15 of Cycle 1; Days 1, 8, and 15 of Cycle 2; Days 1 and 15 of Cycle 3; and Day 1 of Cycle 7. During the maintenance stage after the induction stage, the RNA vaccine was administered on Day 1 of Cycle 13, and once every 24 weeks or 168 days thereafter. See Example 1 for further details.

In the Phase Ia study, 29 patients enrolled in cohorts with doses ranging from 25-100 μg. The most common tumor types were HR+/HER2+ breast, prostate, and ovarian cancer. The median of number of prior therapies was 5 (range 1-17). 34% of patients received prior immunotherapy. Most patients had low PD-L1 expression (97% patients with <5% PD-L1 expression on tumor cells, 93% patients with <5% expression on immune cells). The median number of RNA vaccine doses received was 6; 28% of patients discontinued due to PD prior to completing 6 weeks of therapy. The majority of adverse events (AE) were Grade 1-2. AEs occurring in ≥20% of patients included infusion related reaction (IRR)/cytokine release syndrome (CRS), fatigue, nausea, and diarrhea. IRR/CRS were transient and reversible and presented primarily as Grade 1-2 chills and fever. A single DLT of Grade 3 CRS occurred at the 100 μg dose level. No patients discontinued the RNA vaccine due to AEs. RNA vaccine induced pulsatile release of pro-inflammatory cytokines with each dose, consistent with the innate immune agonist activity of the RNA. RNA vaccine-induced neoantigen specific T cell responses were observed in peripheral blood in 14 out of 16 (87%) patients by ex vivo ELISPOT or MHC multimer analysis. MHC multimer analysis showed the induction of up to 5% neo-epitope specific CD8 T-cells with memory phenotype in the peripheral blood.

RNA vaccine-induced T cells against multiple neoantigens were detected in post-treatment tumor biopsies. Of 26 patients that underwent at least one tumor assessment, 1 patient (4%) with gastric cancer had a response of CR ongoing for ≥10 months, and 11 patients (42%) had SD.

The RNA vaccine can be manufactured for individual patients with clinically relevant turn-around times. In this study, the RNA vaccine had a manageable safety profile consistent with its mechanism of action, and induced strong neoantigen-specific immune responses in patients with low and intermediate mutational load tumors types.

As further described in Example 1, a first-in-human Phase 1b study of the RNA vaccine in combination with the anti-PD-L1 antibody atezolizumab was also conducted in patients with locally advanced or metastatic solid tumors.

The RNA vaccine was administered as described above. Atezolizumab was administered on day 1 of each 21-day cycle. See Example 1 for further details.

132 patients were enrolled in cohorts with doses ranging from 15 μg to 50 μg of the RNA vaccine in combination with 1200 mg atezolizumab. Most common tumor types were NSCLC, TNBC, melanoma and colorectal cancer (CRC). The median number of prior therapies was 3 (range 1-11). 39% of patients received prior immunotherapy. Most patients had low levels of PD-L1 expression (93% patients with <5% PD-L1 expression on tumor cells, 79% patients with <5% PD-L1 expression on immune cells). The median number of RNA vaccine doses received was 8; 16% of patients discontinued due to PD prior to completing 6 weeks of therapy. The majority of adverse events (AE) were Grade 1-2. AEs occurring in ≥15% of patients included infusion related reaction (IRR)/cytokine release syndrome (CRS), fatigue, nausea, and diarrhea. IRR/CRS were transient and reversible and presented primarily as Grade 1-2 chills and fever. There were no DLTs. Seven patients (5%) discontinued treatment due to AEs related to study drugs.

The RNA vaccine induced a pulsatile release of pro-inflammatory cytokines with each dose, consistent with the innate immune agonist activity of the RNA. RNA vaccine-induced neoantigen-specific T cell responses were observed in peripheral blood in 37 out of 49 (77%) patients by ex vivo ELISPOT or MHC multimer analysis. Induction of up to 6% MHC multimer-stained CD8⁺ T-cells with memory phenotype was observed in peripheral blood. RNA vaccine-induced T cells against multiple neoantigens were detected in post-treatment tumor biopsies. Of 108 patients that underwent at least one tumor assessment, 9 responded (ORR 8%, including 1 CR) and 53 patients had SD (49%).

The RNA vaccine in combination with atezolizumab had a manageable safety profile consistent with the mechanisms of action of the study drugs, and induced significant levels of neoantigen-specific immune responses.

In summary, the Phase Ia and Ib trials described herein were non-registrational signal seeking studies that included patients with melanoma, non-small cell lung cancer, bladder cancer, colorectal cancer, TNBC, renal cancer, head and neck cancer, sarcomas. As shown in Example 1, the studies were designed to enroll both patients with and without prior checkpoint inhibitor regimens. The primary objective of the study was to assess safety (including dose-limiting toxicities), and additional objectives included evaluation of immunogenicity and preliminary assessment of anti-tumor activity. The trial included a Phase 1a (monotherapy) dose escalation, a Phase 1b (combination) dose escalation, and multiple Phase 1b expansion cohorts. Patients received nine doses of the RNA vaccine administered i.v. in weekly and bi-weekly intervals during the induction phase and every eight cycles during the maintenance phase. In the Phase 1b portion of the trial, atezolizumab was administered on day one of each 21-day cycle.

RNA vaccine was manufactured on a per-patient basis including in-house determination of cancer mutation profiles, computational prediction of neoantigens, design, and manufacturing of the vaccine based on liposomally formulated RNA (RNA-LPX). Each vaccine contained up to 20 tumor-specific neoepitopes. Importantly, the manufacturing of the vaccine for individual patients within clinical practice compatible turn-around times was shown to be feasible using clinical biopsies or routine clinical specimens across a range of tumor types including those with low or intermediate tumor mutational burden.

Preliminary clinical results were assessed from 29 patients in the Phase 1a trial and 132 patients in the Phase 1b trial. Phase 1a patients had received a median of 5 prior therapies (range 1-17), and Phase 1b patients had received a median of 3 prior therapies (range 1-11). RNA vaccine, both with and without atezolizumab, had a manageable safety profile with predominantly transient and reversible grade 1 and grade 2 adverse events such as infusion related reaction/cytokine release syndrome manifesting as fever and chills. Analyses with complementary quantitative immunoassays showed that RNA vaccine, both with and without atezolizumab, induced strong neoepitope-specific immune responses, including in patients with tumors of low and intermediate mutational burden. Vaccine-induced neo-antigen specific T cells were detected in post-vaccine biopsies. A best response of stable disease was observed in almost half of RNA vaccine-treated patients, including objective responses in a limited number of patients, including both patients with and without prior checkpoint inhibitor regimens. This indicates a level of clinical activity for the RNA vaccine in combination with atezolizumab, however randomized data is needed to assess the individual contribution of RNA vaccine on top of a checkpoint inhibitor.

Moreover, based on previous studies of an RNA vaccine as an adjunct to surgery in patients with metastatic melanoma, and without wishing to be bound to theory, it is thought that the RNA vaccine is potentially well suited to control metastatic relapses in patients with a lower tumor burden.

### Example 3: Immune Responses Induced by an RNA Vaccine as a Single Agent and in Combination with Atezolizumab in Patients with Locally Advanced or Metastatic Solid Tumors

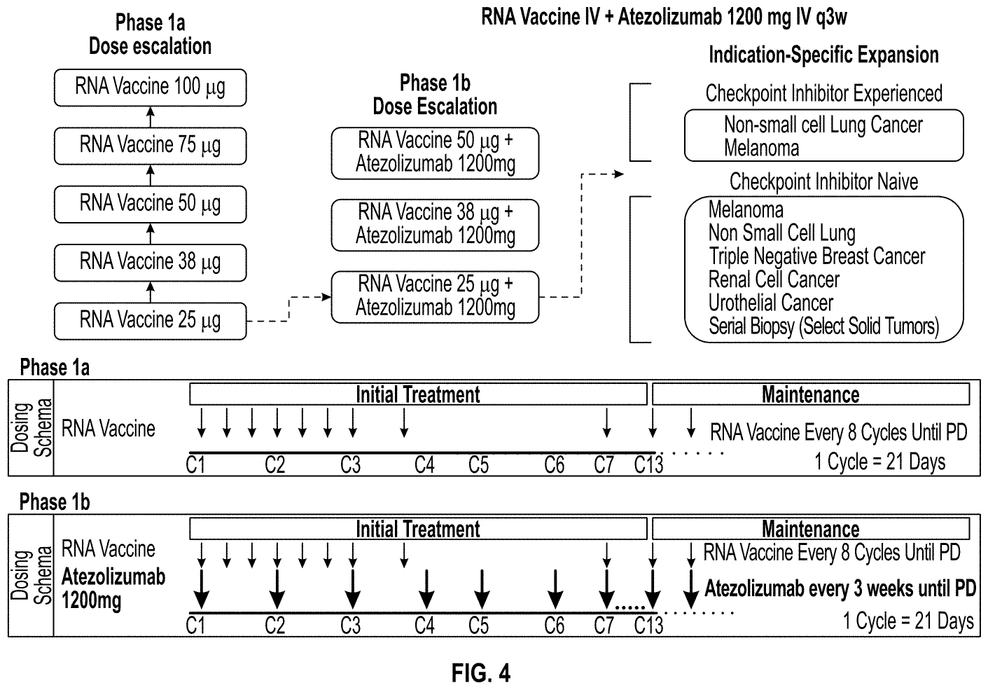

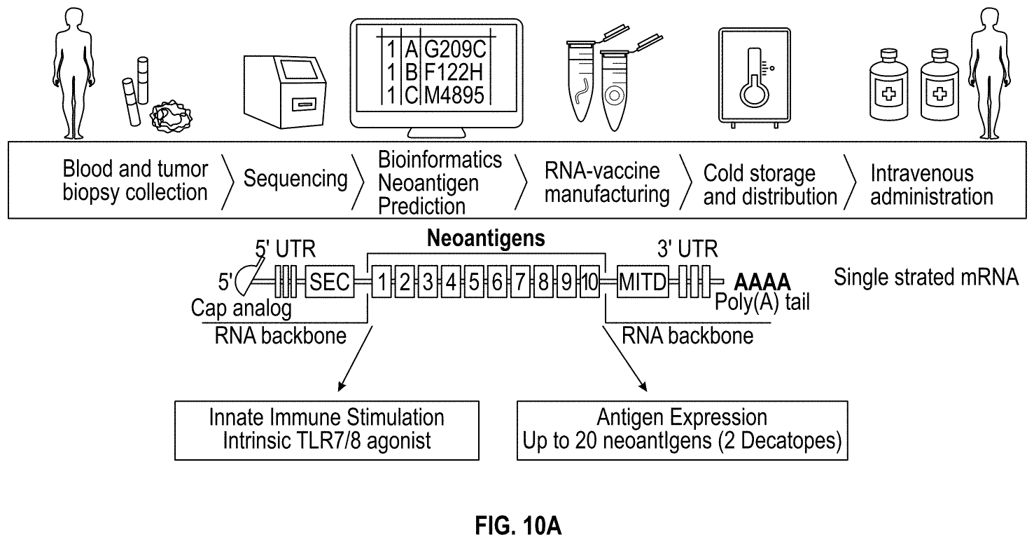

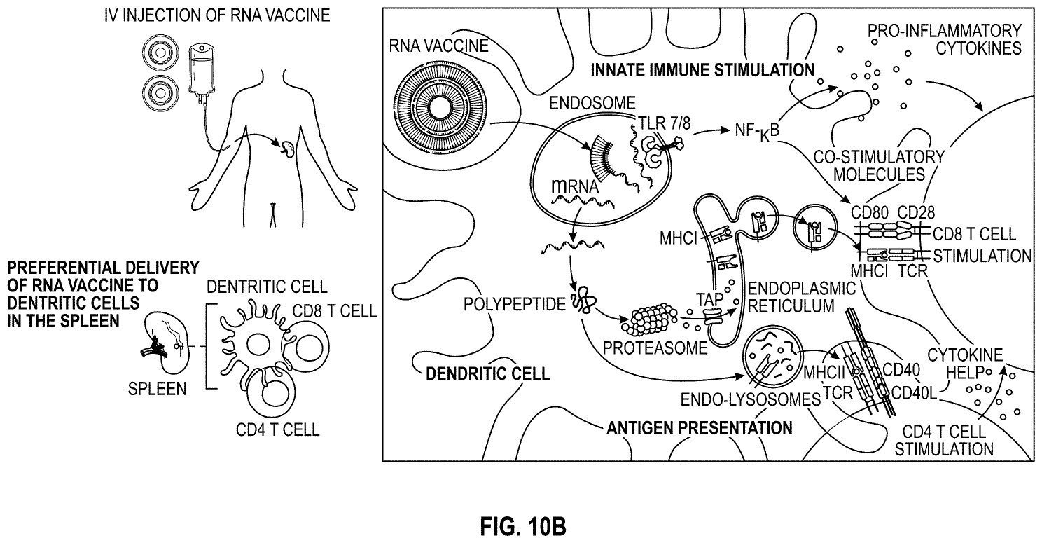

As described in Examples 1 and 2, the first-in-human Phase Ia and Phase Ib studies of an RNA vaccine as a monotherapy (Phase Ia) and in combination with atezolizumab (Phase Ib) were conducted in patients with locally advanced or metastatic solid tumors (FIG. 4). The RNA vaccine was manufactured on a per-patient basis and contained up to 20 tumor-specific neoepitopes (see, e.g., FIG. 10A and Türeci et al (2016) Clin Canc Res, 22(8):1885-96; Vormehr et al (2019) Ann Rev Med, 70:395-407; and Sahin et al (2018) Science, 359(6382):1355-1360). This Example describes the results of experiments evaluating innate and neoantigen-specific immune responses induced by the RNA vaccine alone and in combination with atezolizumab.

### Materials and Methods

### ELISPOT Assay

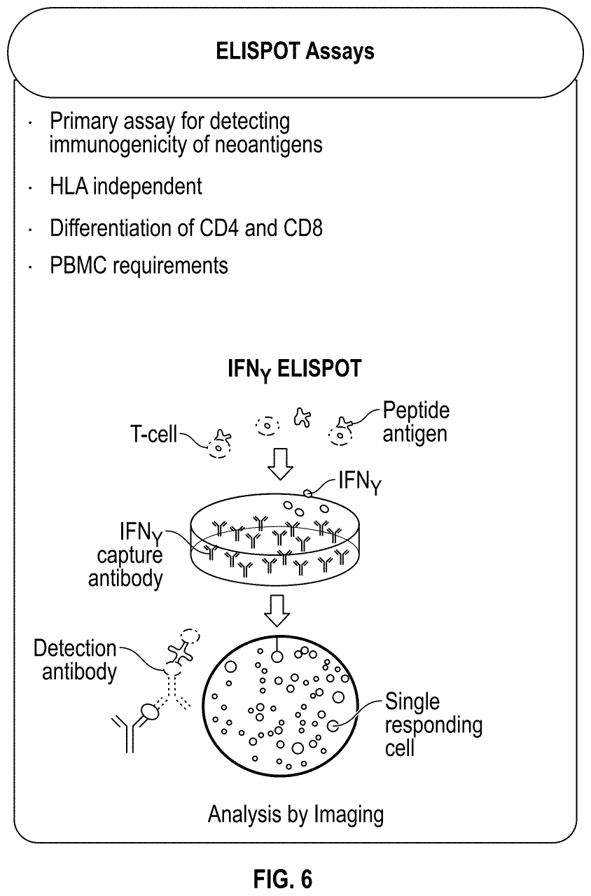

Bulk peripheral blood mononuclear cells (PBMCs) or separated CD8+ T cells and CD4+ T cells were stimulated in vitro with overlapping peptides corresponding to up to 20 individual neoantigen targets in the RNA vaccine. After overnight stimulation, IFNg production was assessed using the ELISPOT method. The number of spots in this assay corresponds to the frequency of neoantigen specific T cells in the PBMCs or in the separated CD8+ T cells and CD4+ T cells. Each neoantigen target was tested in duplicate wells. Internal controls with no neoantigen peptides were used to define positive staining in the assay. Specifically, a positive reaction was designated if the average number of spots in the test wells exceeded 15 and had a statistically significant difference from the control wells. To define RNA vaccine-specific responses, the number of spots from samples obtained after treatment with the RNA vaccine were compared with a baseline sample (before RNA vaccine treatment) for the same neoantigen; a positive hit was defined as positive reaction in the post-treatment sample and negative reaction in the baseline sample, or a two fold increase over the baseline spot count in the post-treatment sample if the baseline sample was also positive. A diagram of the ELISPOT assay methods is provided in FIG. 6.

pMHC Multimer Assay

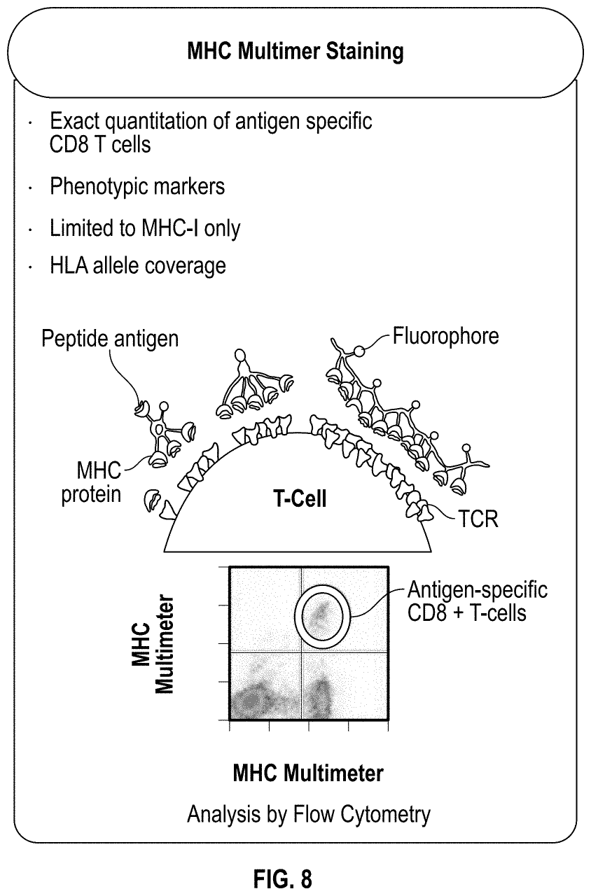

Individual pMHC multimers were designed for each patient based on the patient's HLA class I allele and using peptides derived from predicted epitopes in the neoantigen targets used in the RNA vaccine. Frozen peripheral blood mononuclear cells (PBMCs) were used for fluorescence activated cell sorting (FACS) staining Each sample was stained with multiple pMHC multimers and additional antibodies for defining the phenotypes of neoantigen-specific CD8+ T cells. FACS panels were designed such that each neoantigen had two pMHC mutimers labeled with two different fluorophores (to increase the specificity of the staining). CD8+ T cells were gated among the PBMCs and analyzed for staining with the two pMHC mutimers labeled with two different fluorophores for each neoantigen. In order for any given CD8+ T cell to be called positively stained (i.e., neoantigen-specific), it had to stain positive for both of the pMHC multimers labeled with two different fluorophores and fall in the top right quadrant in the FACS histogram. A diagram of the pMHC multimer staining assay methods is provided in FIG. 8.

### Results

### Innate Immune Responses

The innate immune responses induced by the RNA vaccine as a monotherapy (Phase Ia) or in combination with atezolizumab (Phase Ib) were evaluated by measuring the levels of cytokines (e.g., IFNg or IFNα) in plasma using enzyme-linked immunosorbent assay (ELISA) analyses before the start of treatment and at multiple timepoints after administration of the RNA vaccine and atezolizumab.

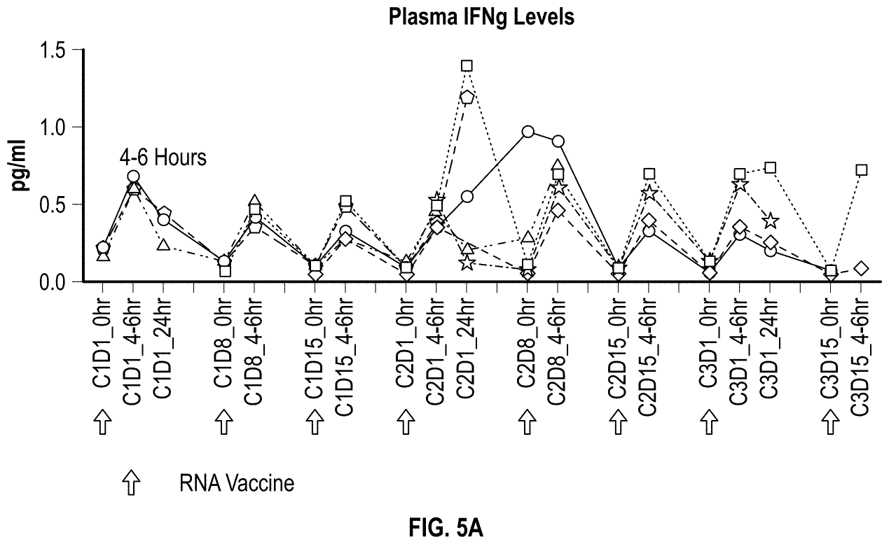

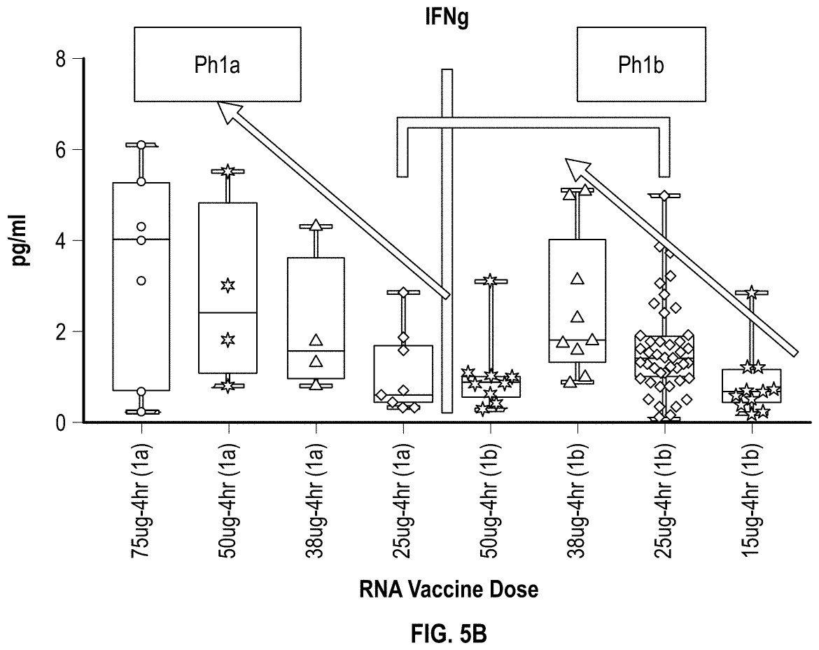

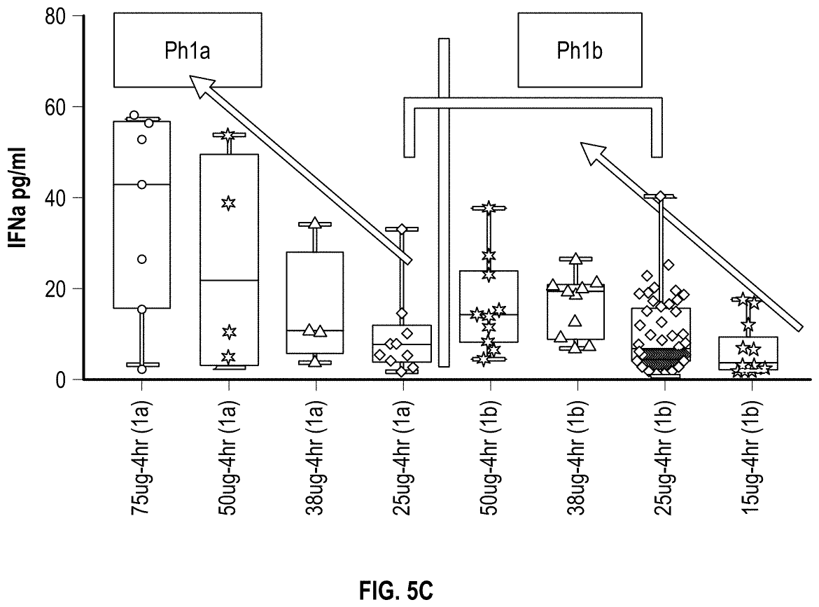

As shown in FIG. 5A for the Phase Ia study, patients administered the RNA vaccine at a dose of 25 μg in the Phase Ia study exhibited a pulsatile rise in plasma IFNg levels (results from five patients are shown). In addition, plasma IFNg levels at 4 hours after each administration of the RNA vaccine increased in a dose dependent manner (FIG. 5B). The levels of IFNa at 4 hours after each administration of the RNA vaccine were also increased in a dose dependent manner (FIG. 5C). Several patients administered the RNA vaccine at a dose of 50 μg received steroids and had a dose reduction to 25 μg.

Cytokine levels were also evaluated at 4 hours after each administration of the RNA vaccine in patients in the Phase Ib study. As shown in FIGS. 5B-5C, plasma IFNg and IFNa levels at 4 hours after each administration of the RNA vaccine were increased in a dose dependent manner.

Overall, these results showed that administration of the RNA vaccine as either a monotherapy or in combination with atezolizumab resulted in a robust and dose-dependent innate immune activation, consistent with the proposed function of the RNA vaccine as an innate immune stimulator through TLR7/8 agonism (see, e.g., FIGS. 10A-10B). In addition, the innate immune response was enhanced by the RNA vaccine and atezolizumab combination compared to the RNA vaccine monotherapy (FIGS. 5B-5C). This effect was most pronounced at the 25 μg RNA vaccine dose. Similar results were observed for other cytokines, including IL-6, and IL-12 (data not shown).

### Neoantigen-Specific Immune Responses

Neoantigen-specific immune responses following administration of the RNA vaccine as a monotherapy (Phase Ia) or in combination with atezolizumab (Phase Ib) were assessed using ex vivo EliSpot assays (FIG. 6) and MHC multimer staining assays (FIG. 8).

EliSpot Assays

Neoantigen-specific immune responses following administration of the RNA vaccine as a monotherapy (Phase Ia) or in combination with atezolizumab (Phase Ib) were first assessed using ex vivo IFNg EliSpot assays at Cycle 4, Day 1 (FIG. 6).

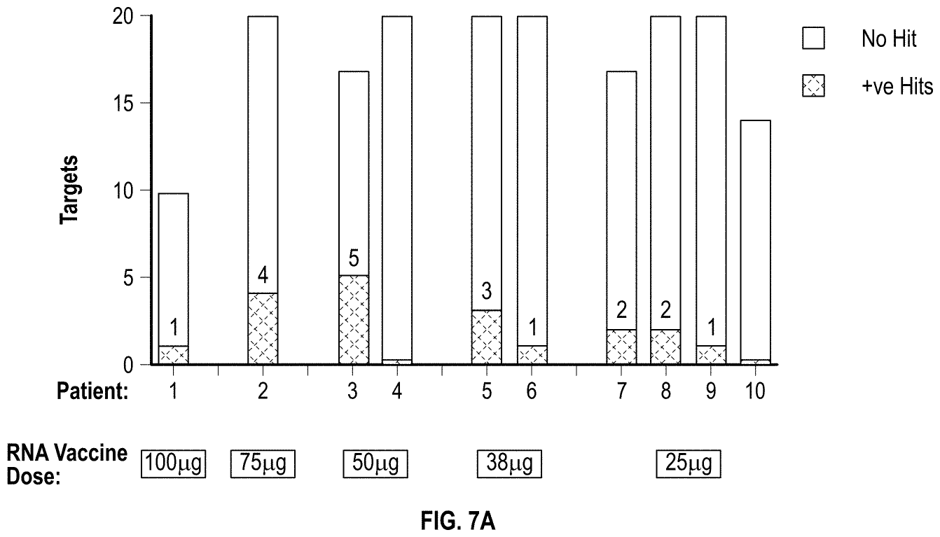

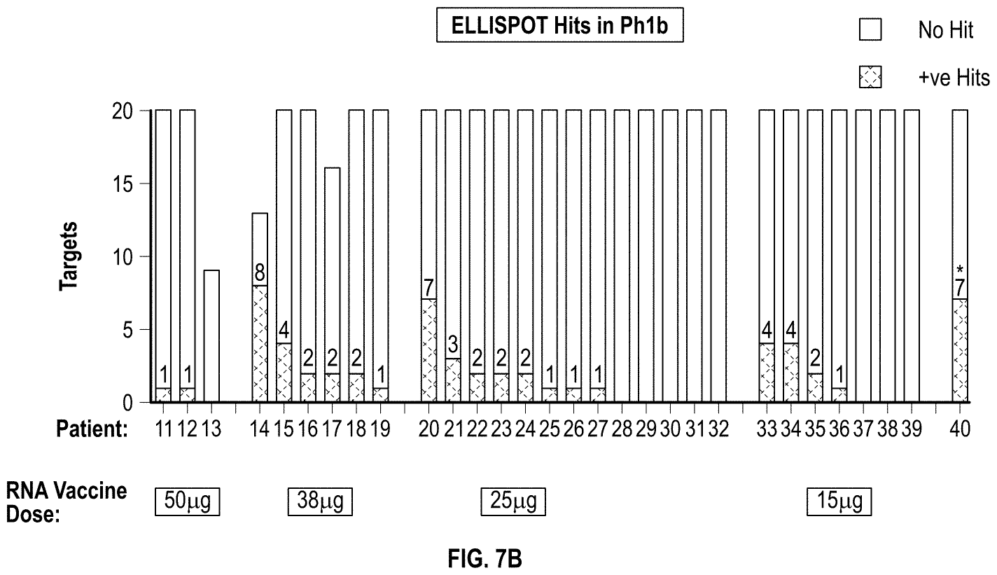

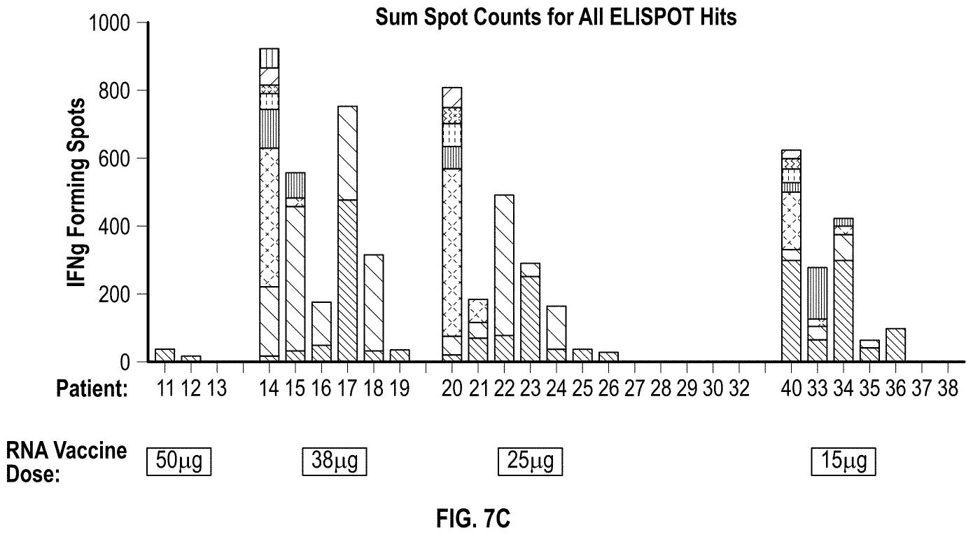

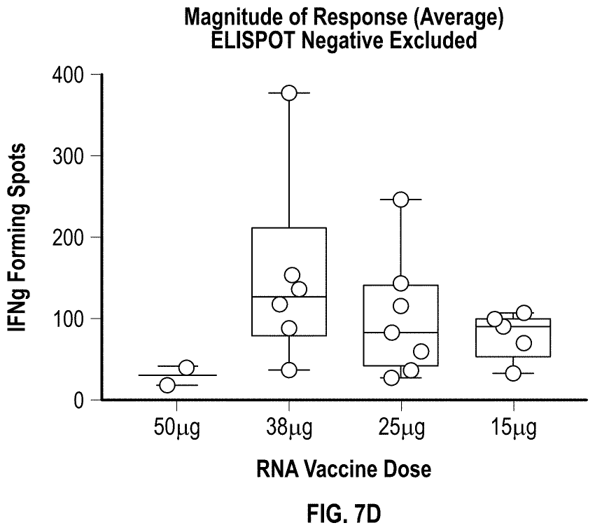

As shown in FIG. 7A, patients administered the RNA vaccine as a monotherapy (Phase Ia) exhibited neoantigen-specific immune responses that varied in breadth (i.e., the number of antigens that induced an immune response). For example, patient 1, who was administered the RNA vaccine at a dose of 100 μg, showed a neoantigen-specific immune response against one out of ten antigens (10%). In another example, patient 2, who was administered the RNA vaccine at a dose of 75 μg, showed a neoantigen-specific immune response against four out of twenty antigens (20%).

Patients administered the RNA vaccine in combination with atezolizumab (Phase Ib) also exhibited neoantigen-specific immune responses that varied in breadth (i.e., the number of antigens that induced an immune response). For example, as shown in FIG. 7B, patient 11, who was administered the RNA vaccine at a dose of 50 μg, showed a neoantigen-specific immune response against one out of twenty antigens (5%). In another example, patient 20, who was administered the RNA vaccine at a dose of 25 μg, showed a neoantigen-specific immune response against seven out of twenty antigens (35%).

The magnitudes of the observed neoantigen-specific immune responses were also determined for patients in the Phase Ib study. As shown in FIG. 7C, the number of IFNg forming spots for each neoantigen that induced an immune response varied. Patient 27 did not have any positive neoantigen hits by EliSpot assay but did exhibit one positive neoantigen hit by pMHC multimer staining assay (see below). The data shown for patients 20 and 14 in FIG. 7C includes both CD4 and CD8 spots for each neoantigen hit. The data for patient 12 shows CD4+ T cell responses. In addition, the median magnitudes of the observed immune responses varied among patients within and across RNA vaccine doses, as shown in FIG. 7D and Table 3.

TABLE 3
Magnitudes of neoantigen-specific immune responses
observed in patients in the Phase Ib study.

| RNA vaccine | | | | |
|---|---|---|---|---|
| dose: | 50 μg | 38 μg | 25 μg | 15 μg |
| Number of | 2 | 6 | 7 | 5 |
| patients | | | | |
| Median number | 29 | 127.2 | 81.5 | 88.71 |
| of IFNg forming | | | | |
| spots | | | | |
| Mean number of | 29 | 152.4 | 101.7 | 78.89 |
| IFNg forming | | | | |
| spots | | | | |

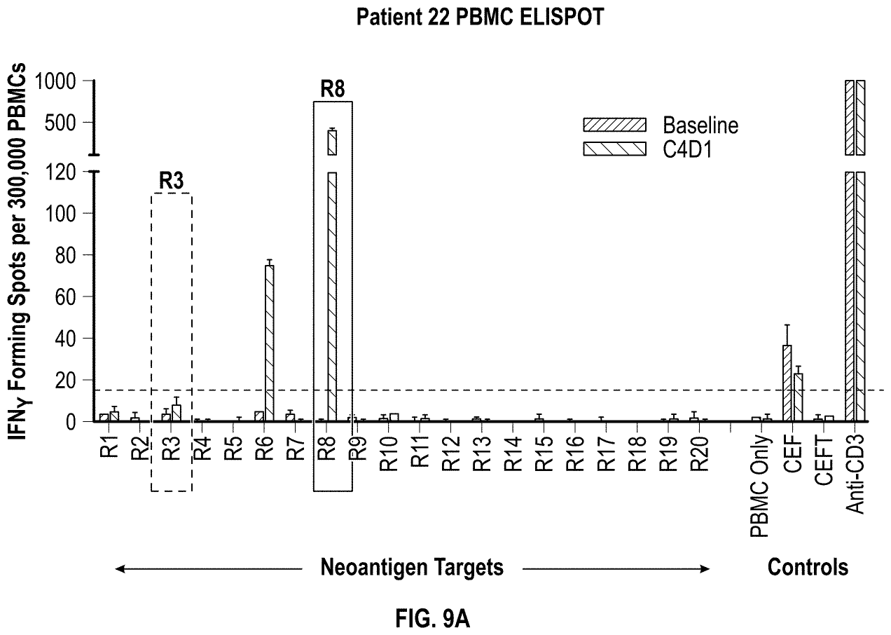

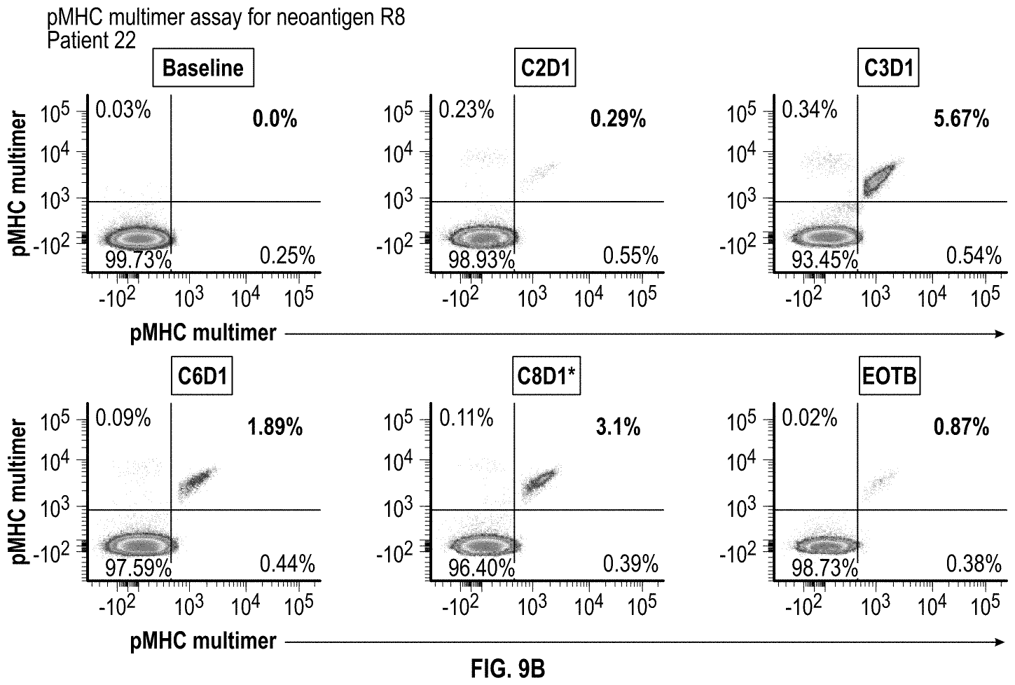

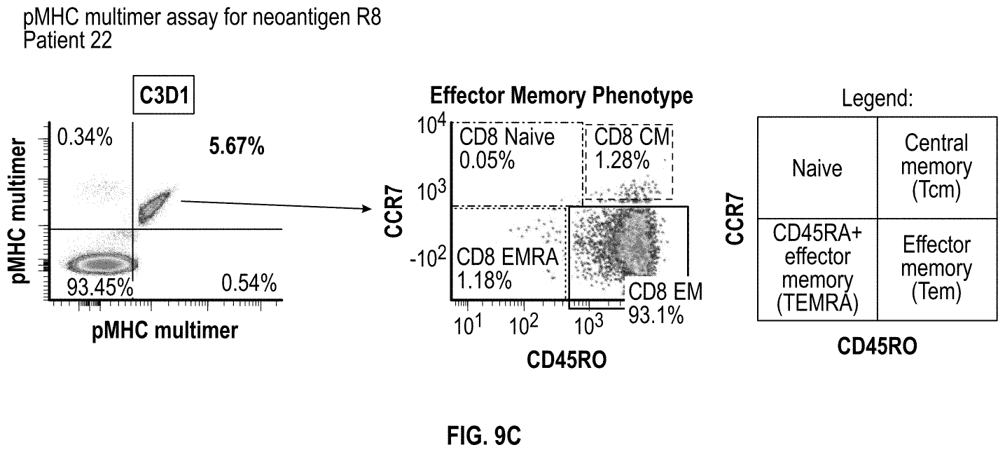

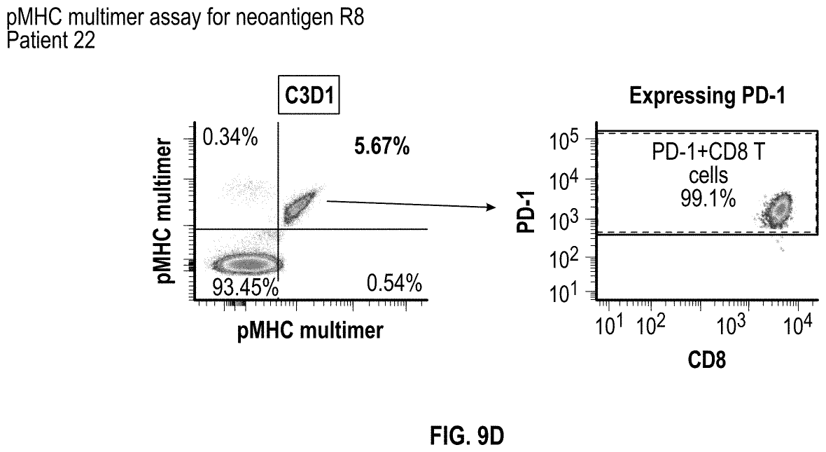

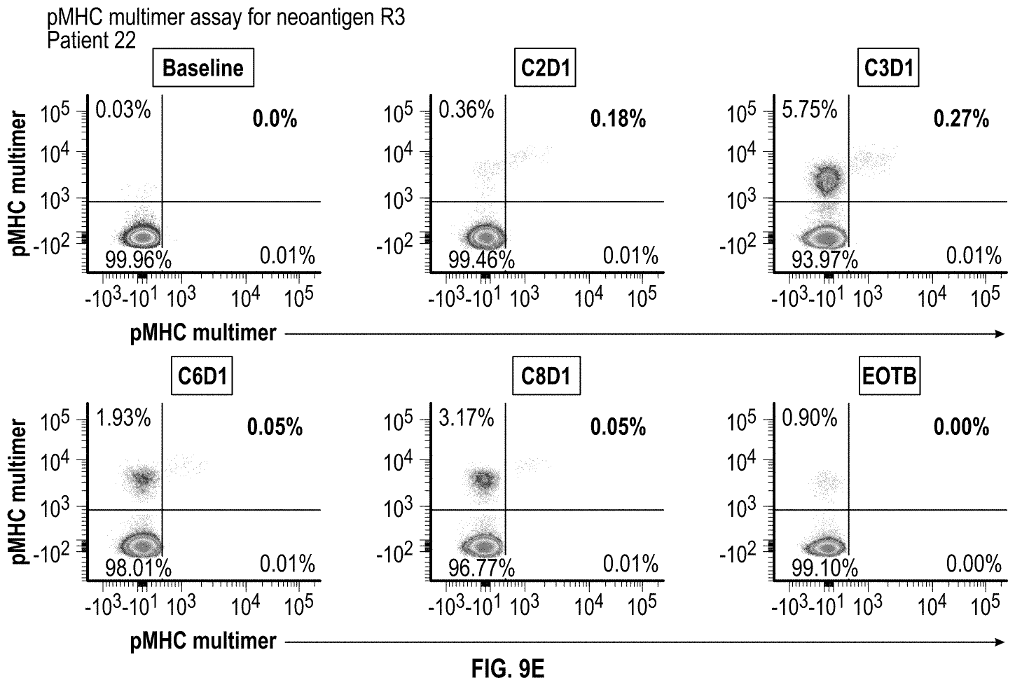

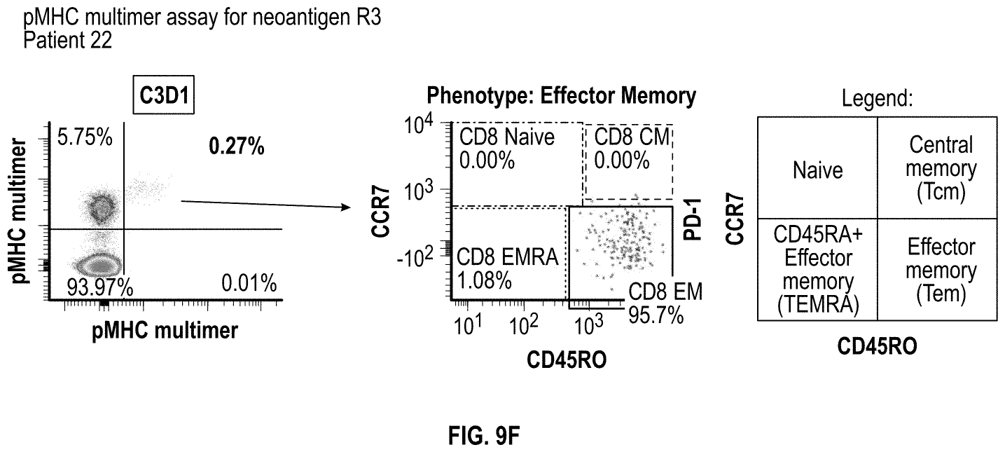

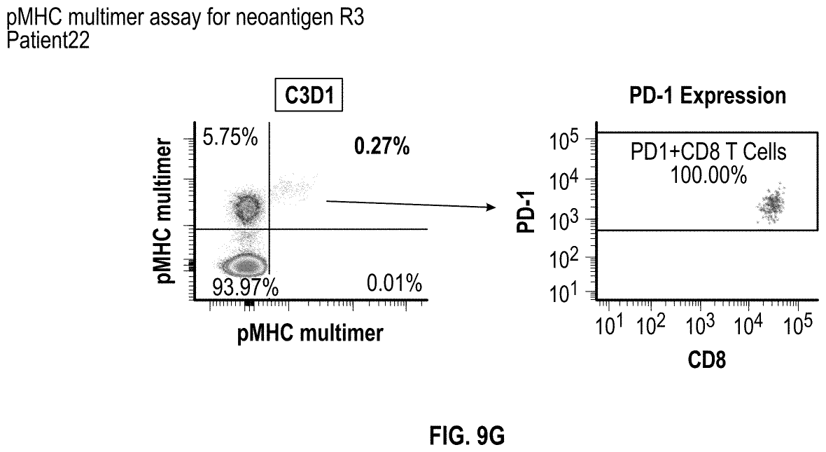

In one example, IFNg EliSpot assays performed on bulk PBMCs obtained from a CIT-naïve triple-negative breast cancer patient administered the RNA vaccine at a dose of 25 μg in combination with atezolizumab (Phase Ib; patient 22) showed that antigens R6 and R8 resulted in neoantigen-specific immune responses at Cycle 4, Day 1 (FIG. 9A). In contrast, neoantigen R3 was not detected as a positive hit.

pMHC Multimer Assays

Neoantigen-specific CD8+ T cell responses in patient 22 (see FIG. 9A) were also evaluated using fully quantitative peptide MHC (pMHC) multimer staining assays (FIG. 8).

As shown in FIG. 9B, in agreement with the bulk PBMC EliSpot assays shown in FIG. 9A, a CD8+ T cell response specific for neoantigen R8 was detected using pMHC multimer staining assays. The kinetics of the neoantigen-specific CD8+ T cell immune responses suggested that the peak response (i.e., about 5.67% neoantigen-specific CD8+ T cells) occurred at between about 3 to about 6 vaccine doses, and that the immune response was boosted by a dose at C7D1 (see, C8D1 in FIG. 9B). An analysis of the markers expressed by the neoantigen-specific CD8+ T cell population at Cycle 3, Day 1 showed that the population included CD45+RA+ effector memory cells (TEMRA; 1.18%), central memory cells (Tcm; 1.28%), and effector memory cells (Tem; 93.10%) (FIG. 9C). In addition, 99.1% of the neoantigen-specific CD8+ T cell population was PD-1+(FIG. 9D).

In contrast to the results observed with neoantigen R8, while the bulk PBMC EliSpot assays shown in FIG. 9A failed to detect neoantigen R3 as a positive hit, a CD8+ T cell response specific for neoantigen R3 was detected using pMHC multimer assays (FIG. 9E). The kinetics of the neoantigen-specific CD8+ T cell immune responses against neoantigen R3 suggested that the peak response (i.e., about 0.27% neoantigen-specific CD8+ T cells) also occurred at between about 3 to about 6 vaccine doses. An analysis of the markers expressed by the neoantigen-specific CD8+ T cell population at Cycle 3, Day 1 showed that the population included CD45+RA+ effector memory cells (TEMRA; 1.08%), and effector memory cells (Tem; 95.7%) (FIG. 9F). In addition, 100.00% of the neoantigen-specific CD8+ T cell population was PD-1+ (FIG. 9G).

Overall, these results showed that neoantigen-specific T cell responses were detected with EliSpot assays as well as pMHC multimer assays following administration of the RNA vaccine in combination with atezolizumab, and that the magnitude of CD8+ T cells induced by the RNA vaccine can reach up to >5% in peripheral blood (e.g., up to about 6%). In addition, the results suggested that pMHC multimer assays have greater sensitivity compared to EliSpot assays. Furthermore, the neoantigen-specific immune responses induced by the RNA vaccine included CD8+ T cells that had high expression of PD-1 (i.e., PD-1+) and primarily had an effector memory phenotype. These results suggested that the RNA vaccine resulted in long-lasting neoantigen-specific immune responses.

### Discussion

The results presented in this Example showed that administration of the RNA vaccine as either a monotherapy or in combination with atezolizumab resulted in robust innate immune activation as well as neoantigen-specific immune responses. These results are consistent with the proposed mechanism of action of the RNA vaccine, which, as shown in FIGS. 10A-10B, is believed to act through both innate immune stimulation (e.g., intrinsic TLR7/8 agonism) as well as by stimulating neoantigen-specific T cell responses (e.g., CD4+ and CD8+ T cell responses) following presentation of neoantigens by dendritic cells (see, e.g., Kranz et al (2016) Nature, 16; 534(7607):396-401).

### Example 4: Additional Results from a Phase Ia Study of an RNA Vaccine as a Single Agent in Patients with Locally Advanced or Metastatic Solid Tumors

This Example provides additional safety and efficacy results of the Phase Ia study of an RNA vaccine as a monotherapy in patients with locally advanced or metastatic solid tumors described in Examples 1-3.

As shown in FIG. 4, patients in the Phase Ia dose escalation study were administered the RNA vaccine in doses ranging from 25 μg to 100 μg (25 μg, 38 μg, 50 μg, 75 μg, and 100 μg). During initial treatment (induction stage), the RNA vaccine was administered in 21-day cycles. During initial treatment (induction stage), the RNA vaccine was administered on Days 1, 8, and 15 of Cycle 1; Days 1, 8, and 15 of Cycle 2; Days 1 and 15 of Cycle 3; and Day 1 of Cycle 7. During the maintenance stage after the initial treatment, the RNA vaccine was administered on Day 1 of Cycle 13, and every 8 cycles thereafter (i.e., once every 24 weeks thereafter or once every 168 days thereafter), until disease progression.

### Patient Demographics and Disease Characteristics

As shown in Table 4, the median age of patients in this study was 59 years and most patients were female (65%). 55% of patients had an ECOG performance status of 1 and 45% of patients had an ECOG performance status of 0. The most common tumor types were breast cancer (HER2+ or HR+), prostate cancer, ovarian cancer, bone sarcoma, endometrial cancer, gastric cancer, and soft tissue sarcoma. Patients had received a median number of 5 prior systemic therapies for metastatic disease and 32% of patients had received a prior treatment with a checkpoint inhibitor. In addition, 90% of patients had PD-L1 expression in <5% of tumor-infiltrating immune cells and tumor cells and 10% of patients had PD-L1 expression in ≥5% tumor-infiltrating immune cells or tumor cells.

TABLE 4
Patient demographics and disease characteristics.

| | Dose Escalation (N = 31) |
|---|---|
| Median (range) age, years | 59 (21-77) |
| Female, n (%) | 20 (65) |
| ECOG PS, n (%) | |
| 0 | 14 (45) |
| 1 | 17 (55) |
| Most common tumor types, n (%) | |
| Breast cancer (HER2+ or HR+) | 6 (19) |
| Prostate cancer | 5 (16) |
| Ovarian cancer | 4 (13) |
| Bone sarcoma | 4 (13) |
| Endometrial cancer | 2 (7) |
| Gastric cancer | 2 (7) |
| Soft tissue sarcoma | 2 (7) |
| Median (range) number of prior systemic | 5 (1-17) |
| therapies for metastatic disease, n | |
| Prior checkpoint inhibitors, n (%) | 10 (32) |
| PD-L1 (Ventana SP142), n (%) | |
| <5% IC and TC | 28 (90) |
| ≥5% IC or TC | 3 (10) |

IC = tumor-infiltrating immune cell;
TC = tumor cell;
ECOG PS = Eastern Cooperative Oncology Group performance status;
HER = human epidermal growth factor receptor;
HR = hormone receptor;
PD-L1 = programmed death-ligand 1.

### Exposure and Disposition

As shown in Table 5, the median duration of treatment for all patients in Phase Ia was 43 days. During treatment, one dose-limiting toxicity (DLT) was observed at the 100 μg RNA vaccine dose (Grade 3 cytokine release syndrome). RNA vaccine dose reductions occurred in one patient who was administered the RNA vaccine at a dose of 38 μg. Overall, 29 patients have discontinued treatment, 12 due to cross over to Phase Ib, 11 due to disease progression, and 5 due to study withdrawal. Eight patients in the study discontinued treatment due to disease progression prior to completing 6 weeks of therapy.

TABLE 5
Patient exposure and disposition during treatment.

| | RNA Vaccine IV Dose | | | | | | | | | | | |
|---|---|---|---|---|---|---|---|---|---|---|---|---|
| | 25 μg | | 38 μg | | 50 μg | | 75 μg | | 100 μg | | Total | |
| | (n = 13) | | (n = 5) | | (n = 4) | | (n = 8) | | (n = 1) | | (N = 31) | |
| DLT, n (%) | 0 | | 0 | | 0 | | 0 | | 1 | (100)^{a} | 1 | (3) |
| RNA vaccine dose | 0 | | 1 | (20) | 0 | | 0 | | 0 | | 1 | (3) |
| reduction, n (%) | | | | | | | | | | | | |
| Median (range) | 43 | (1-123) | 42 | (15-128) | 40 | (15-254) | 40 | (9-69) | 56 | (56-56) | 43 | (1-254) |
| treatment duration, | | | | | | | | | | | | |
| days | | | | | | | | | | | | |
| Continuing | 0 | | 1 | (20) | 1 | (25) | 0 | | 0 | | 2 | (7) |
| treatment, n (%) | | | | | | | | | | | | |
| Discontinued study | 13 | (100) | 4 | (80) | 3 | (75) | 8 | (100) | 1 | (100) | 29 | (94) |
| treatment, n (%) | | | | | | | | | | | | |

Reasons for treatment discontinuation, n (%)

| | | | | | | | | | | | | |
|---|---|---|---|---|---|---|---|---|---|---|---|---|
| Disease progression | 4 | (31) | 1 | (20) | 1 | (25) | 5 | (62) | 1 | (100) | 12 | (39) |

| | | | | | | | | | | | |
|---|---|---|---|---|---|---|---|---|---|---|---|
| Crossover^{b} | 5 | (38) | 2 | (40) | 2 | (50) | 2 | (25) | 0 | 11 | (35) |

| Death | 0 | | 0 | | 0 | 0 | 0 | 0 | |
|---|---|---|---|---|---|---|---|---|---|
| AE | 0 | | 0 | | 0 | 0 | 0 | 0 | |
| Withdrawal by subject | 4 | (31) | 1 | (20) | 0 | 0 | 0 | 5 | (16) |

| Other | 0 | | 0 | 0 | | 1 | (12) | 0 | 1 | (3) |
|---|---|---|---|---|---|---|---|---|---|---|
| Discontinued | 4 | (31) | 0 | 2 | (50) | 2 | (25) | 0 | 8 | (26) |
| treatment due to | | | | | | | | | | |
| disease progression | | | | | | | | | | |
| prior to completing 6 | | | | | | | | | | |
| weeks of therapy, n | | | | | | | | | | |
| (%) | | | | | | | | | | |

^{a}DLT event was Grade 3 cytokine release syndrome (CTCAE v5.0);
^{b}Phase Ia patients with disease progression or loss of clinical benefit may crossover to combination therapy in Phase Ib;
AE = adverse event; DLT = dose-limiting toxicity.

### Safety

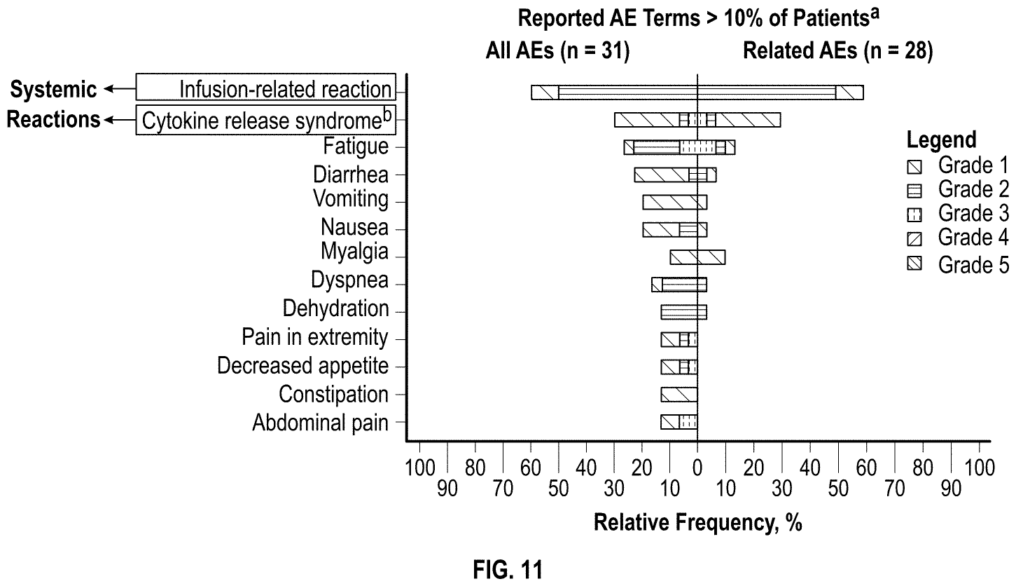

FIG. 11 provides a summary of the most common AEs occurring in >10% of patients. The most common study treatment-related AEs occurring in >10% of patients were systemic reactions, including infusion related reactions and cytokine release syndrome. Other AEs occurring in >10% of patients included fatigue, diarrhea, vomiting, nausea, myalgia, dyspnea, dehydration, pain in extremity, decreased appetite, constipation, and abdominal pain. A serious adverse event (SAE) of malignant neoplasm progression was reported in 16% of patients (data not shown).

Most systemic reactions occurred within about 2-4 hours post-infusion of the RNA vaccine and resolved within about 1-2 hours. Table 6 provides an overview of individual signs and symptoms of systemic reactions occurring in ≥5% of patients. Most events of hypotension and hypoxia were Grade 2, except for a DLT event which had symptoms of Grade 3 hypotension and Grade 3 hypoxia.

TABLE 6
Individual signs and symptoms of systemic
reactions (CRS/IRR/ILI) in ≥5% of patients.

| | RNA Vaccine Dose | | | | | | | | | |
|---|---|---|---|---|---|---|---|---|---|---|
| | 25 μg | | 38 μg | 50 μg | | 75 μg | | 100 μg | All Patients | |
| n (%) | (n = 13) | | (n = 5) | (n = 4) | | (n = 8) | | (n = 1) | (N = 31) | |
| Chills | 8 | (62) | 4 (80) | 4 | (100) | 8 | (100) | 1 (100) | 25 | (81) |
| Pyrexia | 6 | (46) | 2 (40) | 3 | (75) | 5 | (63) | 1 (100) | 17 | (55) |
| Nausea | 3 | (23) | 2 (40) | 4 | (100) | 3 | (38) | 0 | 12 | (39) |
| Headache | 3 | (23) | 1 (20) | 1 | (25) | 1 | (13) | 0 | 6 | (19) |

| | | | | | | | | | |
|---|---|---|---|---|---|---|---|---|---|
| Vomiting | 3 | (23) | 1 (20) | 1 | (25) | 0 | 0 | 5 | (16) |

| Hypotension | 0 | | 1 (20) | 0 | 2 | (25) | 1 (100) | 4 | (13) |
|---|---|---|---|---|---|---|---|---|---|
| Hypoxia | 0 | | 1 (20) | 0 | 1 | (13) | 1 (100) | 3 | (10) |
| Myalgia | 2 | (15) | 0 | 0 | 1 | (13) | 0 | 3 | (10) |

| | | | | | | | | | |
|---|---|---|---|---|---|---|---|---|---|
| Tachycardia | 0 | 0 | 1 | (25) | 2 | (25) | 0 | 3 | (10) |

| | | | | | | | | |
|---|---|---|---|---|---|---|---|---|
| Neck pain | 1 | (8) | 1 (20) | 0 | 0 | 0 | 2 | (7) |
| Sinus | 1 | (8) | 1 (20) | 0 | 0 | 0 | 2 | (7) |
| tachycardia | | | | | | | | |

| | | | | | | | | |
|---|---|---|---|---|---|---|---|---|
| Tremor | 0 | 1 (20) | 1 | (25) | 0 | 0 | 2 | (7) |

CRS = cytokine release syndrome (CTCAE v.5); IRR = infusion-related reaction; ILI = influenza-like illness.

Overall, the safety results showed that the RNA vaccine was generally well-tolerated, with treatment-related AEs being primarily transient systemic reactions that manifested as low-grade cytokine release syndrome, infusion related reactions, or flu-like symptoms. Systemic reactions were transient and generally manageable in the outpatient setting. The maximum tolerated dose (MTD) was not reached.

### Innate Immune Responses

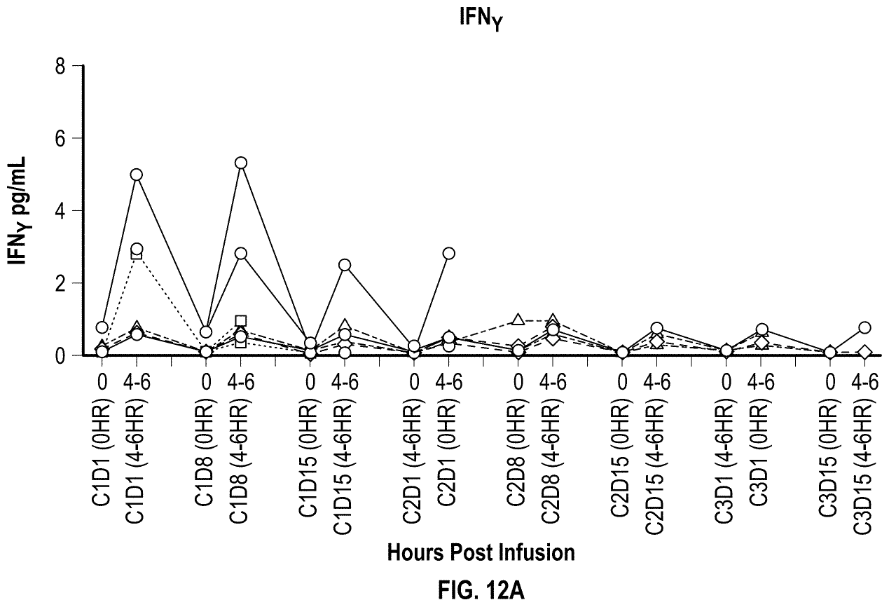

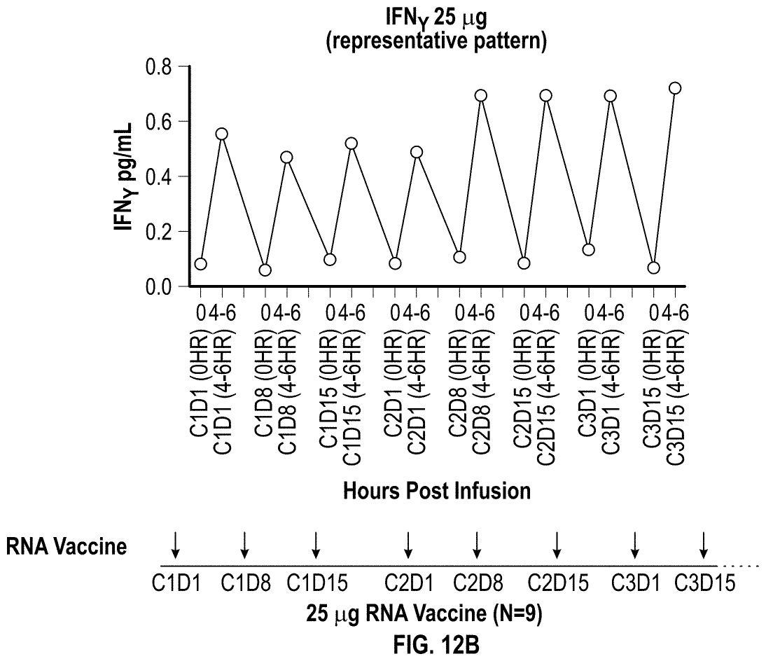

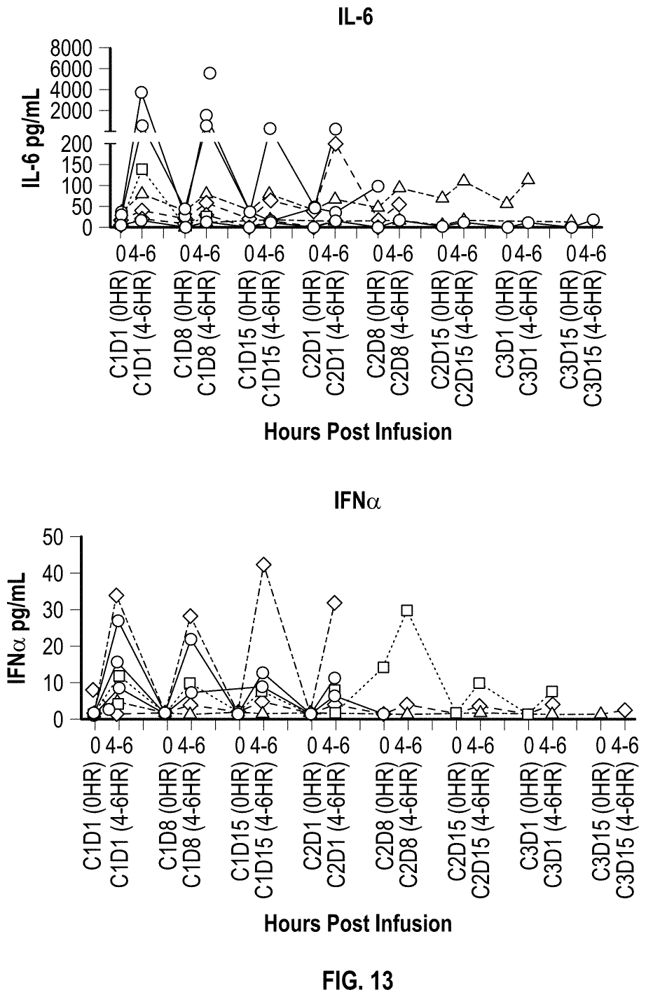

Treatment with the RNA vaccine as a monotherapy induced pulsatile releases of pro-inflammatory cytokines measured in plasma with each RNA vaccine dose. For example, as shown in FIGS. 12A-12B, patients administered the RNA vaccine at a dose of 25 μg exhibited pulsatile releases of IFNγ after each RNA vaccine dose. A similar pattern of pulsatile releases of IL-6 and IFNα in patients administered the RNA vaccine at a dose of 25 μg was also observed (FIG. 13). The observed RNA vaccine-induced pulsatile release of pro-inflammatory cytokines was consistent with the proposed innate immune agonist activity of the RNA vaccine.

### Neoantigen Specific Immune Responses

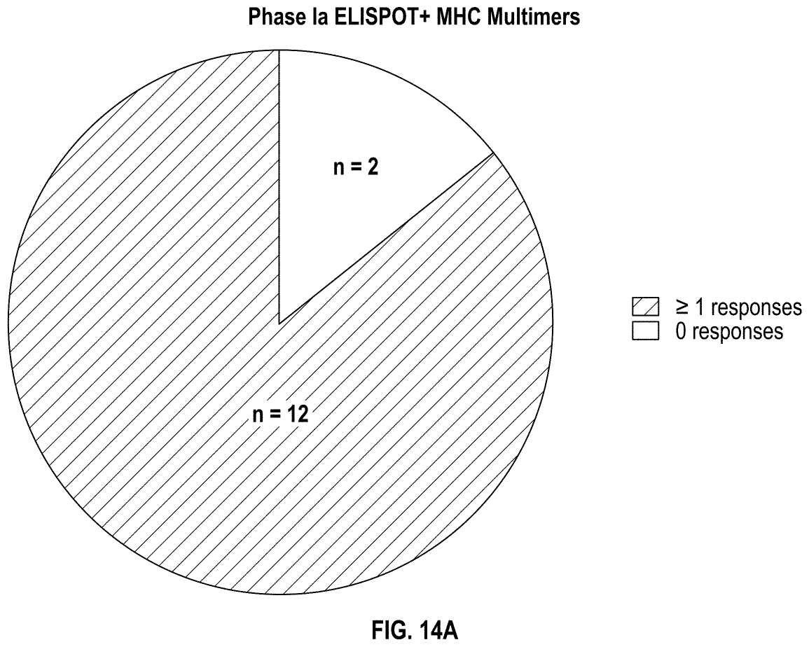

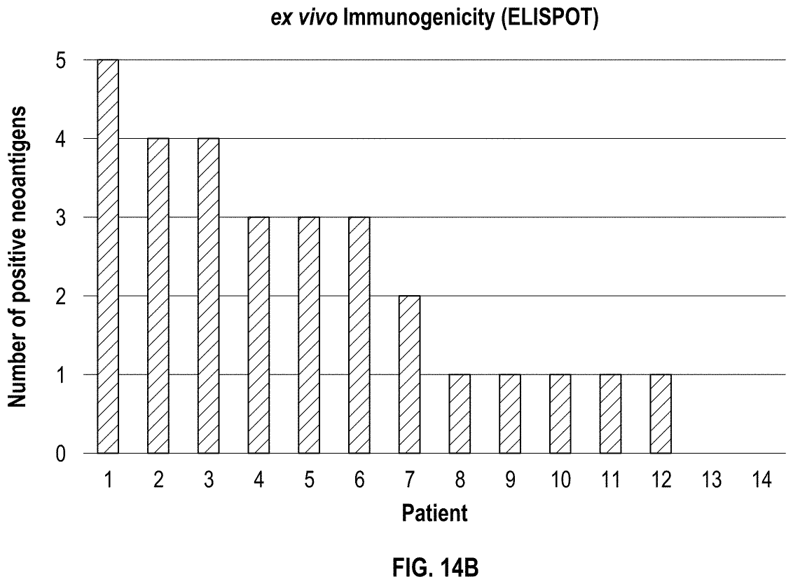

Ex vivo neoantigen-specific T cell responses were detected by EliSpot assays (see, e.g., FIG. 6) and MHC Multimer Staining assays (see, e.g., FIG. 8) in 86% of evaluated patients (FIG. 14A). The median number of neoantigen-specific responses in patients was 2 (range of 1-5) (FIG. 14B).

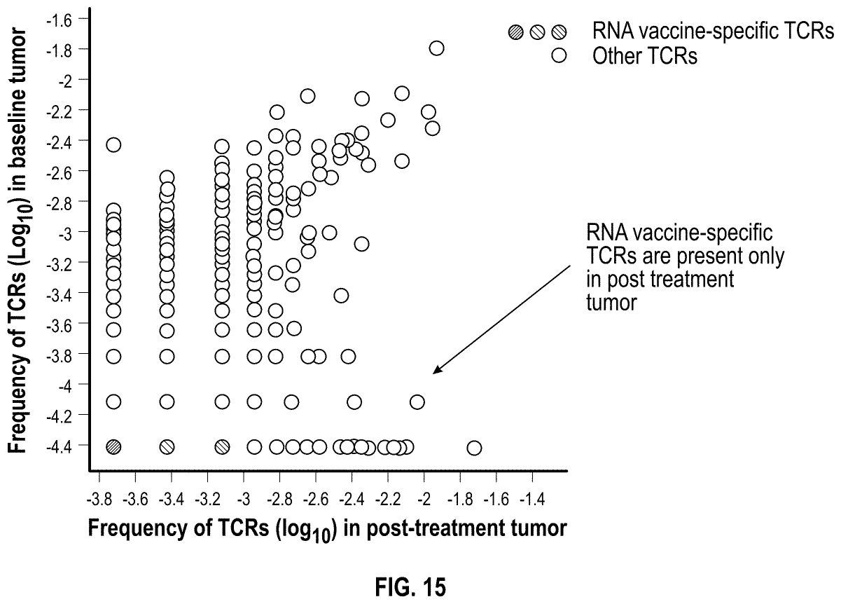

An analysis of T cell receptors by T cell receptor sequencing in a tumor of a prostate cancer patient treated with the RNA vaccine at a dose of 75 μg showed that neoantigen-specific T cells were present in the tumor only after treatment with the RNA vaccine (FIG. 15). These results suggested that the RNA vaccine induced infiltration of T cells stimulated by the RNA vaccine into the tumor.

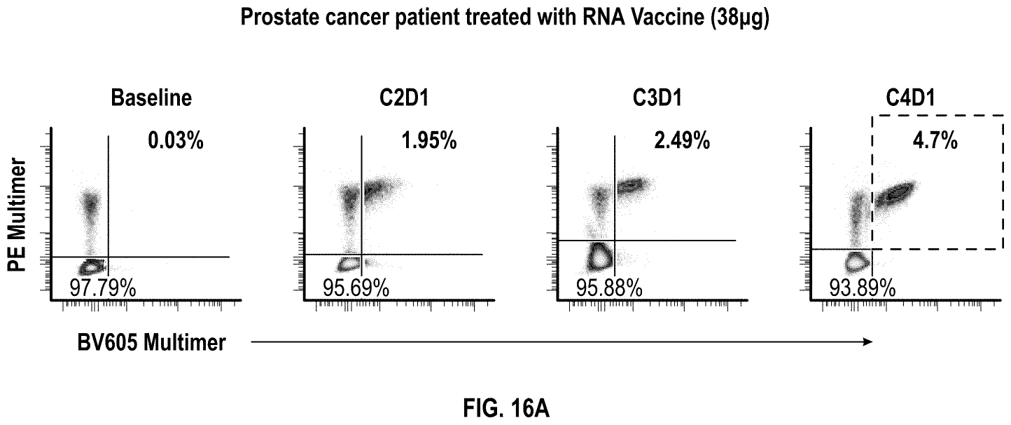

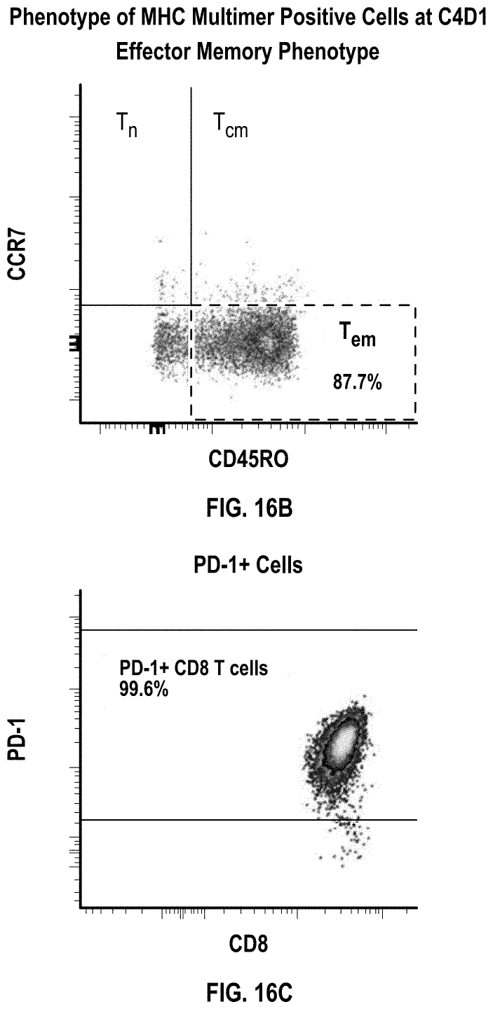

Neoantigen-specific CD8+ T cell responses in a prostate cancer patient treated with the RNA vaccine at a dose of 38 μg were analyzed over time in peripheral blood using fully quantitative peptide MHC (pMHC) multimer staining assays (FIG. 8). As shown in FIG. 16A, CD8+ neoantigen-specific T cells in peripheral blood increased overtime, reaching 4.7% at Cycle 4, Day 1. An analysis of the markers expressed by the neoantigen-specific CD8+ T cell population at Cycle 4, Day 1 revealed that 87.7% of those cells had an effector memory T cell phenotype (Tem; FIG. 16B), and that 99.6% of the cells were PD-1+(FIG. 16C).

The observed RNA vaccine-induced neoantigen-specific immune responses were consistent with the proposed function of the RNA vaccine as a stimulator of neoantigen presentation.

### Clinical Activity

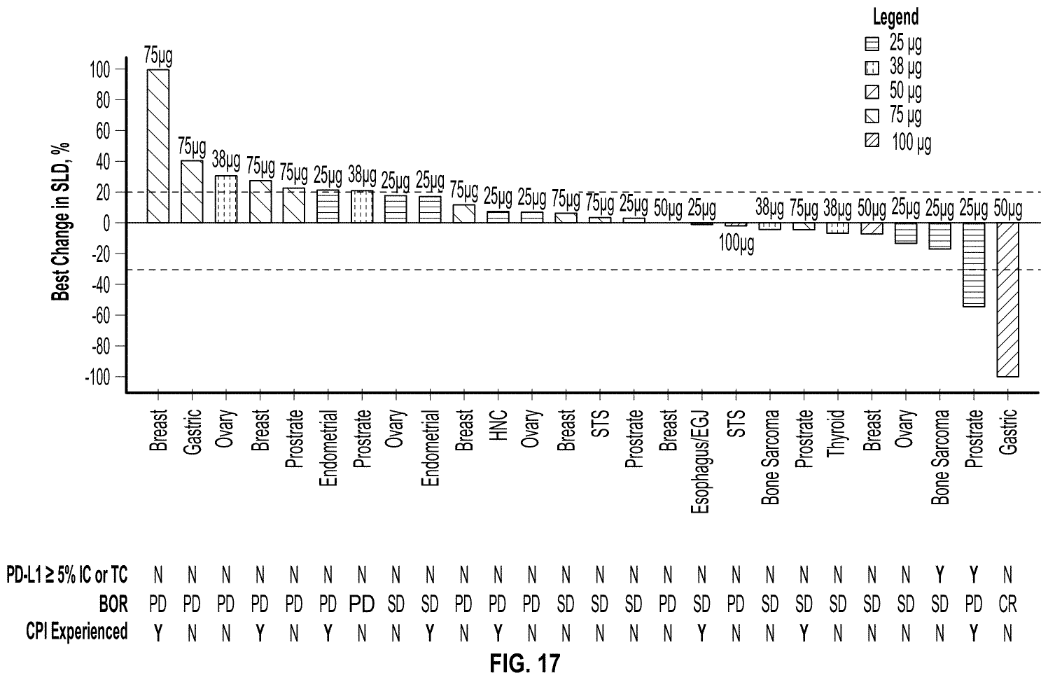

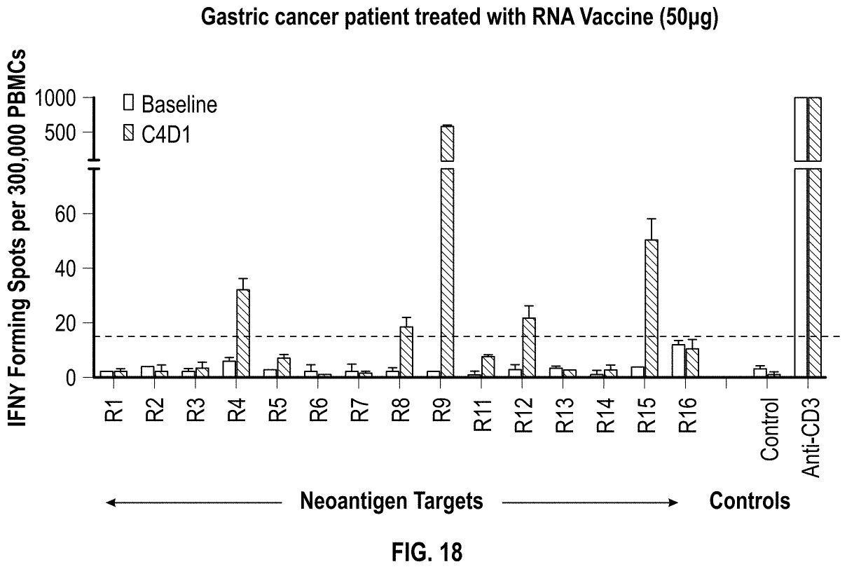

FIG. 17 provides a summary of clinical responses observed in patients treated with the RNA vaccine as a monotherapy and the best percent change in the sum of longest diameters (SLD) from baseline. One patient with gastric cancer treated with the RNA vaccine at a dose of 50 μg exhibited a complete response (CR). This patient had received 3 prior lines of therapy (not including a checkpoint inhibitor) before being administered the RNA vaccine and has been followed up for 1.5 years with continuing RNA vaccine treatment. As shown in FIG. 18, this patient exhibited neoantigen-specific immune responses measured by IFNγ EliSpot assays against antigens R4, R8, R9, R12, and R15 at Cycle 4, Day 1 of the study.

### Discussion

The results described in this Example showed that the RNA vaccine administered as a monotherapy at doses ranging from 25 μg-100 μg was generally well tolerated. Immune monitoring during treatment showed that the RNA vaccine induced pulsatile release of pro-inflammatory cytokines with each dose administered, neoantigen-specific T cell immune responses, and infiltration of stimulated T cells into the tumor of one patient. In addition, clinical efficacy results showed that the RNA vaccine resulted in a complete response in one patient. Overall, these results are consistent with the proposed dual mechanism of action of the RNA vaccine as a stimulator of innate immune responses and neoantigen presentation (see, e.g., FIGS. 10A-10B).

### Example 5: Additional Results from a Phase Ib Study of an RNA Vaccine in Combination with Atezolizumab in Patients with Locally Advanced or Metastatic Solid Tumors

This Example provides additional safety and efficacy results of the Phase Ib study of an RNA vaccine administered in combination with atezolizumab in patients with locally advanced or metastatic solid tumors described in Examples 1-3.

As shown in FIG. 4, patients in the Phase Ib study were administered the RNA vaccine in doses of 15 μg, 25 μg, 38 μg, or 50 μg in combination with 1200 mg atezolizumab. The Phase Ib study included a dose escalation phase for the RNA vaccine doses and an expansion phase in which patients with the indicated checkpoint inhibitor naïve or experienced tumor types were administered the RNA vaccine in combination with atezolizumab. During initial treatment (induction stage), the RNA vaccine was administered on Days 1, 8, and 15 of Cycle 1; Days 1, 8, and 15 of Cycle 2; Days 1 and 15 of Cycle 3; and Day 1 of Cycle 7. During the maintenance stage after the initial treatment, the RNA vaccine was administered on Day 1 of Cycle 13, and every 8 cycles thereafter (i.e., once every 24 weeks thereafter or once every 168 days thereafter), until disease progression. Atezolizumab was administered on Day 1 of each of Cycles 1-12, on Day 1 of Cycle 13, and every 3 weeks thereafter (i.e., every 21 days thereafter), until disease progression (see FIG. 4). Each cycle was 21 days.

### Patient Demographics and Disease Characteristics

As shown in Table 7, in the dose escalation phase, the median age of patients was 57.5 years and 56.6% of patients were male. 50% of patients had an ECOG performance status of 0 and 50% of patients had an ECOG performance status of 1. The most common tumor types in the dose escalation phase were colon cancer (30%), rectal cancer (16.7%), renal cell cancer (10%), and triple negative breast cancer (10%). The median number of prior systemic therapies for metastatic disease was 4 (range: 1-9), with 43.3% of patients having received a prior therapy with a checkpoint inhibitor. 80% of patients had PD-L1 expression in <5% of tumor-infiltrating immune cells and tumor cells, and 16.7% of patients had PD-L1 expression in ≥5% of tumor-infiltrating immune cells or tumor cells.

TABLE 7
Patient demographics and disease
characteristics in the dose escalation phase.

| | Dose Escalation |
|---|---|
| | Total |
| | (n = 30) |
| Median age (range), years | 57.5 (35-77) |
| Male, n (%) | 17 (56.6) |
| ECOG PS, n (%) | |
| 0 | 15 (50) |
| 1 | 15 (50) |
| Most common tumor types, n (%) | |
| Colon cancer | 9 (30.0) |
| NSCLC | — |
| Melanoma | 5 (16.7) |
| Rectal cancer | 3 (10.0) |
| RCC | 3 (10.0) |
| TNBC | — |
| UC | |
| Median number (range) of prior systemic | 4 (1-9) |
| therapies for metastatic disease, n | |
| Prior checkpoint inhibitor, n (%) | 13 (43.3) |
| PD-L1 (Ventana SP142), n (%) | |
| <5% IC and TC | 24 (80.0) |
| ≥5% IC or TC | 5 (16.7) |
| Missing | 1 (3.3) |

NSCLC = non-small cell lung cancer;
RCC = renal cell cancer;
TNBC = triple negative breast cancer;
UC = urothelial cancer;
CPI = checkpoint inhibitor;
IC = tumor-infiltrating immune cell;
TC = tumor cell.

As shown in Table 8, in the expansion phase, the median age was 61.5 years for patients previously treated with a checkpoint inhibitor (CPI-experienced) and 57.5 years for CPI-naïve patients. 59.5% of CPI-experienced patients and 43.1% of CPI-naïve patients were male. 45.2% of CPI-experienced patients and 52.8% of CPI-naïve patients had an ECOG performance status of 0, and 54.8% of CPI-experienced patients and 47.2% of CPI-naïve patients had an ECOG performance status of 1. The most common tumor types in CPI-experienced patients were non-small cell lung cancer (71.4%) and melanoma (19%). The most common tumor types in CPI-naïve patients were non-small cell lung cancer (13.9%), melanoma (12.5%), renal cell cancer (33.3%), and urothelial cancer (13.9%). CPI-experienced patients had received a median of 3 prior systemic therapies for metastatic disease, while CPI-naïve patients had received a median of 2 prior systemic therapies for metastatic disease. 50% of CPI-experienced patients had PD-L1 expression in <5% of tumor-infiltrating immune cells and tumor cells, and 28.6% of patients had PD-L1 expression in ≥5% of tumor-infiltrating immune cells or tumor cells. 75% of CPI-naïve patients had PD-L1 expression in <5% of tumor-infiltrating immune cells and tumor cells, and 13.9% of patients had PD-L1 expression in ≥5% of tumor-infiltrating immune cells or tumor cells.

TABLE 8
Patient demographics and disease
characteristics in the expansion phase.

| | Expansion | |
|---|---|---|
| | CPI Experienced | CPI Naïve |
| | (n = 42) | (n = 72) |
| Median age (range), years | 61.5 (36-82) | 57.5 (29-79) |
| Male, n (%) | 25 (59.5) | 31 (43.1) |
| ECOG PS, n (%) | | |
| 0 | 19 (45.2) | 38 (52.8) |
| 1 | 23 (54.8) | 34 (47.2) |
| Most common tumor types, n (%) | | |
| Colon cancer | — | — |
| NSCLC | 30 (71.4) | 10 (13.9) |
| Melanoma | 8 (19.0) | 9 (12.5) |
| Rectal cancer | — | — |
| RCC | — | 9 (12.5) |
| TNBC | — | 24 (33.3) |
| UC | — | 10 (13.9) |
| Median number (range) of prior | 3 (1-10) | 2 (1-11) |
| systemic | | |
| therapies for metastatic disease, n | | |
| Prior checkpoint inhibitor, n (%) | 42 (100) | 0 |
| PD-L1 (Ventana SP142), n (%) | | |
| <5% IC and TC | 21 (50.0) | 54 (75.0) |
| ≥5% IC or TC | 12 (28.6) | 10 (13.9) |
| Missing | 9 (21.4) | 8 (11.1) |

NSCLC = non-small cell lung cancer;
RCC = renal cell cancer;
TNBC = triple negative breast cancer;
UC = urothelial cancer;
CPI = checkpoint inhibitor;
IC = tumor-infiltrating immune cell;
TC = tumor cell.

### Exposure and Disposition

Table 9 provides a summary of treatment exposure and patient disposition for patients in the Phase Ib study. The median duration of treatment with the RNA vaccine was 57 days and the median duration of treatment with atezolizumab was 66 days. A total of 6 RNA vaccine dose reductions and one RNA vaccine discontinuation occurred. 76.8% of patients have discontinued both study treatments and 23.2% of patients are continuing treatment. 63.4% of RNA vaccine discontinuations occurred due to disease progression, 3.5% occurred due to death, 5.6% occurred due to adverse events, and 1.4% due to withdrawal by subject. 16.9% of patients discontinued study treatment due to disease progression prior to completing 6 weeks of therapy.

TABLE 9
Patient exposure and disposition.

| | RNA Vaccine IV Dose + | | | | |
|---|---|---|---|---|---|
| | Atezolizumab 1200 mg IV q3w | | | | |
| | 15 μg | 25 μg | 38 μg | 50 μg | Total |
| | (n = 27) | (n = 95) | (n = 11) | (n = 9) | (N = 142) |
| DLT, n (%) | 0 | 0 | 0 | 0 | 0 |
| RNA vaccine dose | 1 | 2 | 1 | 2 | 6 |
| reduction, n (%) | (3.7) | (2.1) | (9.1) | (22.2) | (4.2) |
| Median (range) | 65 | 57 | 64 | 36 | 57 |
| treatment duration | (8- | (1- | (35- | (1- | (1- |
| with RNA vaccine, | 253) | 400) | 441) | 253) | 441) |
| days | | | | | |
| Median (range) | 104 | 64 | 106 | 22 | 66 |
| treatment duration | (1- | (1- | (21- | (1- | (1- |
| with atezolizumab, | 316) | 462) | 504) | 296) | 504) |
| days | | | | | |
| Continuing treatment, | 9 | 22 | 2 | 0 | 33 |
| n (%) | (33.3) | (23.2) | (18.3) | | (23.2) |
| Discontinued RNA | 0 | 1 | 0 | 0 | 1 |
| vaccine only, n (%) | | (1-1)^{a} | | | (0.7) |
| Discontinued both | 18 | 72 | 9 | 9 | 109 |
| study treatments, n | (66.7) | (75.8) | (81.8) | (100) | (76.8) |
| (%) | | | | | |
| Reasons for RNA | | | | | |
| vaccine | | | | | |
| discontinuation, n (%) | | | | | |
| Disease progression | 15 | 61 | 8 | 6 | 90 |
| | (55.6) | (64.2) | (72.7) | (66.7) | (63.4) |
| Death^{b} | 1 | 4 | 0 | 0 | 5 |
| | (3.7) | (4.2) | | | (3.5) |
| AE | 0 | 5 | 1 | 2 | 8 |
| | | (5.3) | (9.1) | (22.2) | (5.6) |
| Withdrawal by | 1 | 1 | 0 | 0 | 2 (1.4) |
| subject | (3.7) | (1.1) | | | |
| Other | 1 | 2 | 0 | 1 | 4 |
| | (3.7) | (2.1) | | (11.1) | (2.8) |
| Discontinued | 2 | 19 | 1 | 2 | 24 |
| treatment due to | (7.4) | (20.0) | (9.1) | (22.2) | (16.9) |
| disease progression | | | | | |
| prior to completing 6 | | | | | |
| weeks | | | | | |
| of therapy, n (%) | | | | | |

^{a}Patient discontinued atezolizumab at the same time as the RNA vaccine. However, atezolizumab discontinuation information is not available.
^{b}Four deaths were due to malignant neoplasm progression. One death was due to pericardial effusion malignant. None of the deaths were related to study treatment.

### Safety

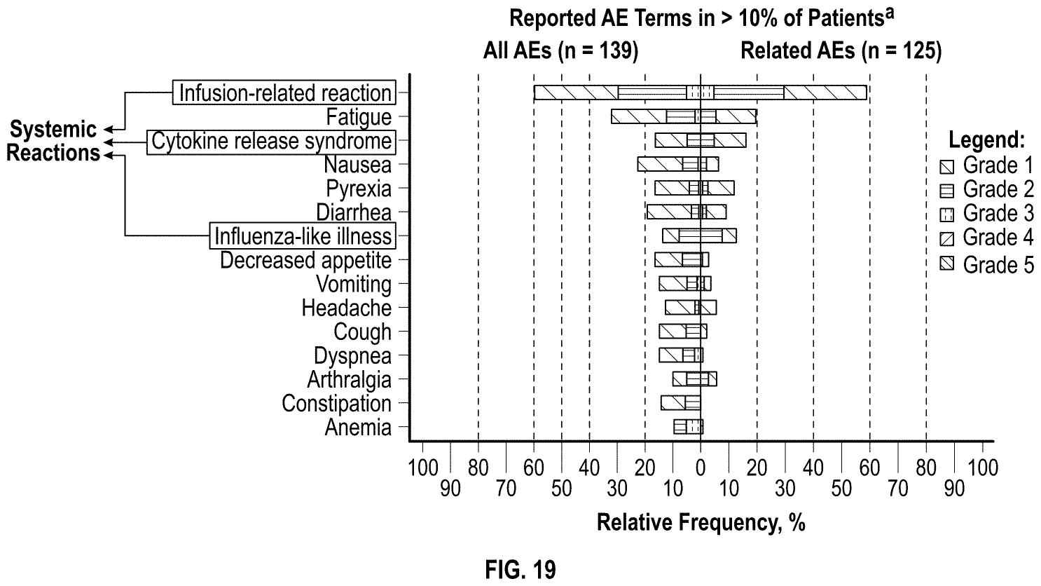

FIG. 19 provides a summary of the most common AEs occurring in >10% of patients in the Phase Ib study. Treatment-related adverse events occurring in >10% of patients were primarily systemic reactions, such as infusion related reaction, cytokine release syndrome, and influenza-like illness. Other AEs occurring in >10% of patients included fatigue, nausea, pyrexia, diarrhea, decreased appetite, vomiting, headache, cough, dyspnea, arthralgia, constipation, and anemia. Serious adverse events of malignant neoplasm progression were reported in 14% of patients (data not shown). No increase in immune-mediated adverse events was observed relative to patients administered the RNA vaccine as a monotherapy in the Phase Ia study described in Examples 1-4 (data not shown).

As shown in Table 10, the median onset time for systemic reactions was 5.7 hours for patients administered the RNA vaccine at a dose of 15 μg, 4.0 hours for patients administered the RNA vaccine at a dose of 25 μg, 4.1 hours for patients administered the RNA vaccine at a dose of 38 μg, and 3.2 hours for patients administered the RNA vaccine at a dose of 50 μg. Systemic reactions resolved within a median time of 1.8 hours or less.

TABLE 10
Median time to onset and resolution of systemic reactions.

| RNA Vaccine IV | Median (range) | Median (range) |
|---|---|---|
| Dose + Atezolizumab | Onset Time, hours | Resolution Time, hours |
| 1200 mg IV | (n = 70) | (n = 57) |
| 15 μg | 5.7 (1.1-11.8) | 1.8 (0.3-5.1) |
| 25 μg | 4.0 (0.7-9.7) | 1.8 (0.1-20.1) |
| 38 μg | 4.1 (2.1-6.1) | 1.5 (0.4-3.3) |
| 50 μg | 3.2 (2.4-5.9) | 1.4 (0.4-1.7) |

Table 11 provides an overview of individual signs and symptoms of systemic reactions occurring in ≥5% of patients.

TABLE 11
Individual signs and symptoms of systemic reactions
(CRS/IRR/ILI) in ≥ 5 patients.
RNA Vaccine IV Dose + Atezolizumab 1200 mg IV

| | | | | | All |
|---|---|---|---|---|---|
| | 15 μg | 25 μg | 38 μg | 50 μg | Patients |
| n (%) | (n = 27) | (n = 95) | (n = 11) | (n = 9) | (N = 142) |
| Pyrexia | 10 (37.0) | 60 (63.2) | 10 (90.9) | 6 (66.7) | 86 (60.6) |
| Chills | 11 (40.7) | 58 (61.1) | 8 (72.7) | 7 (77.8) | 84 (59.2) |
| Nausea | 2 (7.4) | 14 (14.7) | 2 (18.2) | 2 (22.2) | 20 (14.1) |
| Tachycardia | 1 (3.7) | 8 (8.4) | 2 (18.2) | 3 (33.3) | 14 (9.9) |
| Headache | 3 (11.1) | 7 (7.4) | 2 (18.2) | 0 | 12 (8.5) |
| Vomiting | 1 (3.7) | 9 (9.5) | 2 (18.2) | 0 | 12 (8.5) |
| Hypertension | 1 (3.7) | 5 (5.3) | 0 | 2 (22.2) | 8 (5.6) |
| Hypotension | 3 (11.1) | 3 (3.2) | 1 (9.1) | 0 | 7 (4.9) |
| Myalgia | 2 (7.4) | 4 (4.2) | 1 (9.1) | 0 | 7 (4.9) |
| Back pain | 0 | 4 (4.2) | 1 (9.1) | 1 (11.1) | 6 (4.2) |
| Fatigue | 1 (3.7) | 4 (4.2) | 0 | 0 | 5 (3.5) |
| Hypoxia | 0 | 3 (3.2) | 1 (9.1) | 1 (11.1) | 5 (3.5) |

CRS = cytokine release syndrome (CTCAE v.5);
IRR = infusion-related reaction;
ILI = influenza-like illness.

No dose-limiting toxicities were observed and the maximum tolerated dose was not reached. In addition, treatment-related AEs were primarily systemic reactions that manifested as low-grade cytokine release syndrome (CRS), infusion related reactions (IRR), or flu-like symptoms. Overall, systemic reactions were transient, reversible, and manageable in the outpatient setting.

### Innate Immune Responses

Analysis of cytokines in plasma during the study showed that administration of the RNA vaccine in combination with atezolizumab induced pulsatile release of pro-inflammatory cytokines in a manner similar to what was observed for patients in the Phase Ia study, e.g., as described in Example 4 (data not shown).

### Neoantigen Specific Immune Responses

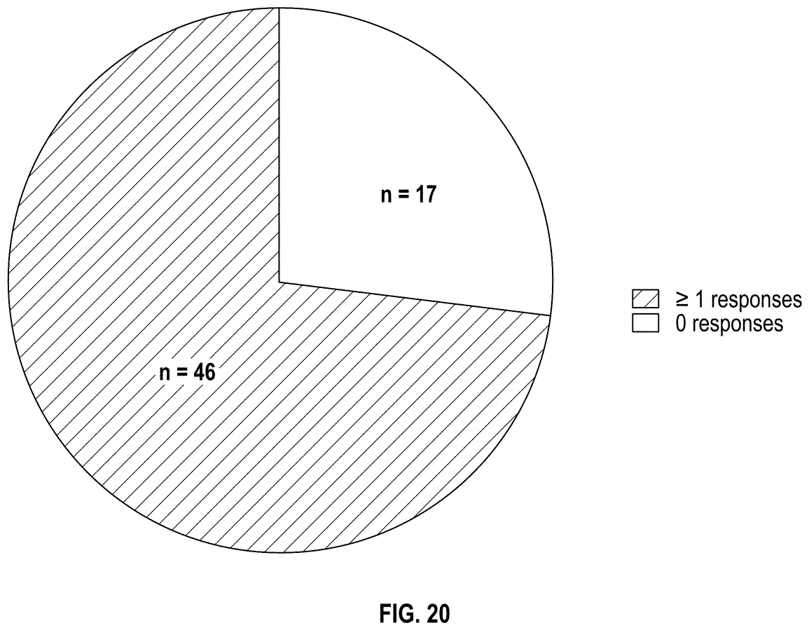

Ex vivo neoantigen specific T cell responses were detected by EliSpot assays (see, e.g., FIG. 6) and MHC Multimer Staining assays (see, e.g., FIG. 8) in about 73% of evaluated patients (n=63) (FIG. 20). The median number of neoantigen-specific responses in patients was 2.6 (range of 1-9). Furthermore, both CD4+ and CD8+ T cell responses were detected in tested patients (n=14) (data not shown).

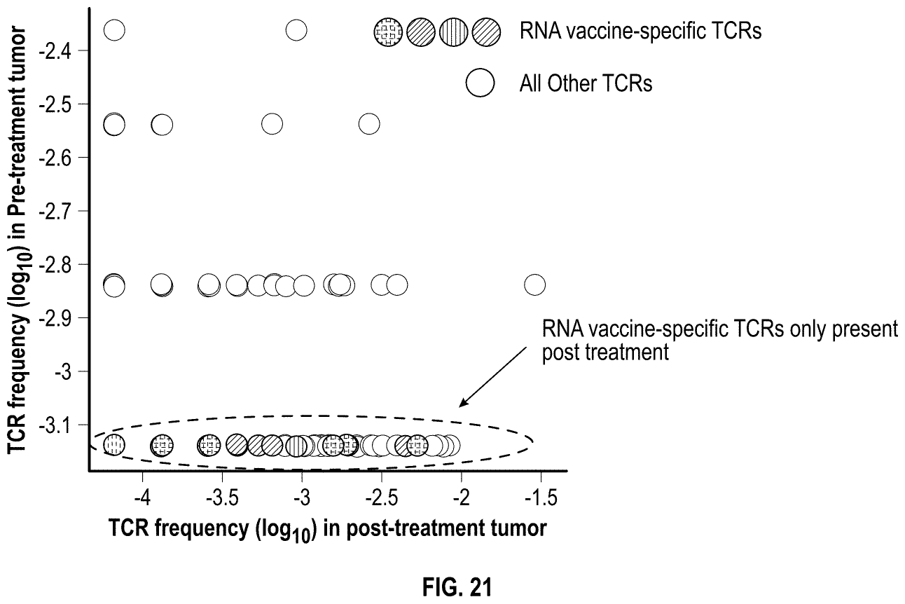

An analysis of T cell receptors by T cell receptor sequencing in a tumor of a rectal cancer patient treated with 1200 mg atezolizumab and the RNA vaccine at a dose of 38 μg showed that neoantigen-specific T cells were present in the tumor only after treatment with the RNA vaccine (FIG. 21). These results suggested that the RNA vaccine induced infiltration of T cells stimulated by the RNA vaccine into the tumor.

Overall, these results showed that the RNA vaccine in combination with atezolizumab induced neoantigen-specific T cell responses in a majority of treated patients.

### Clinical Activity

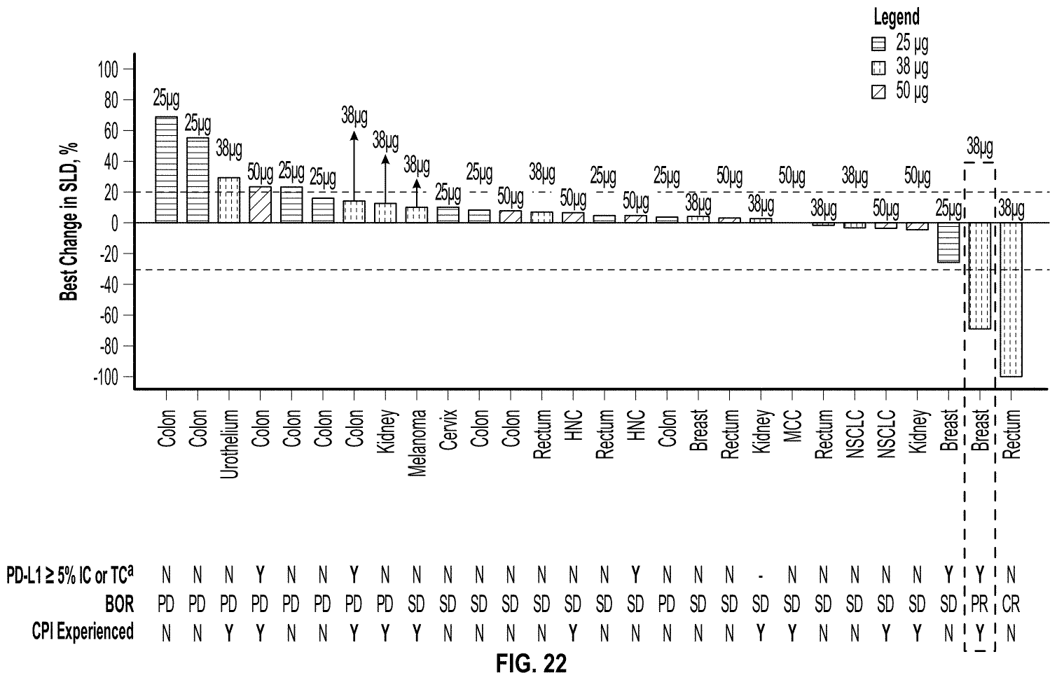

A summary of clinical responses observed in patients treated with the RNA vaccine in combination with atezolizumab is provided in FIG. 22.

One patient with rectum cancer treated with the RNA vaccine at a dose of 38 μg exhibited a complete response (CR). This patient had not been previously treated with a checkpoint inhibitor and did not have PD-L1 expression in ≥5% of tumor infiltrating immune cells or tumor cells as assessed by SP142 Ventana assay.

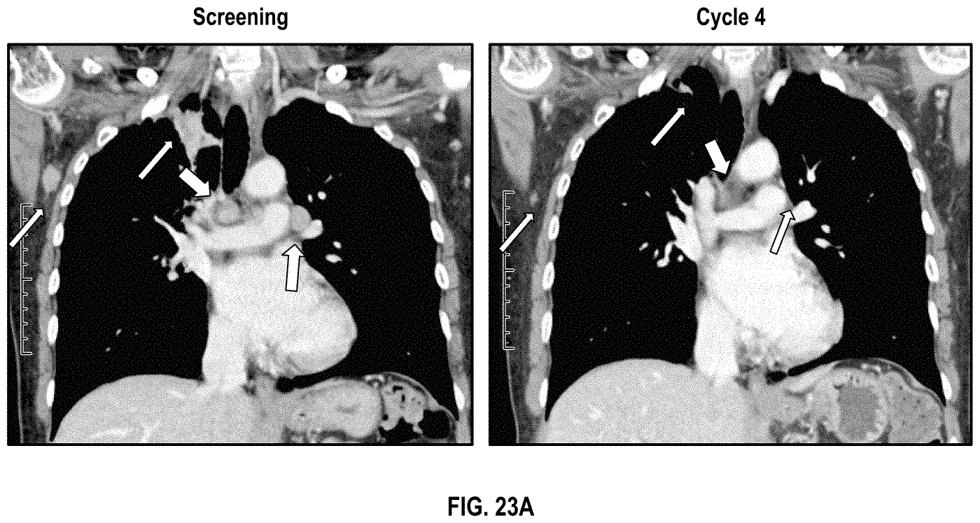

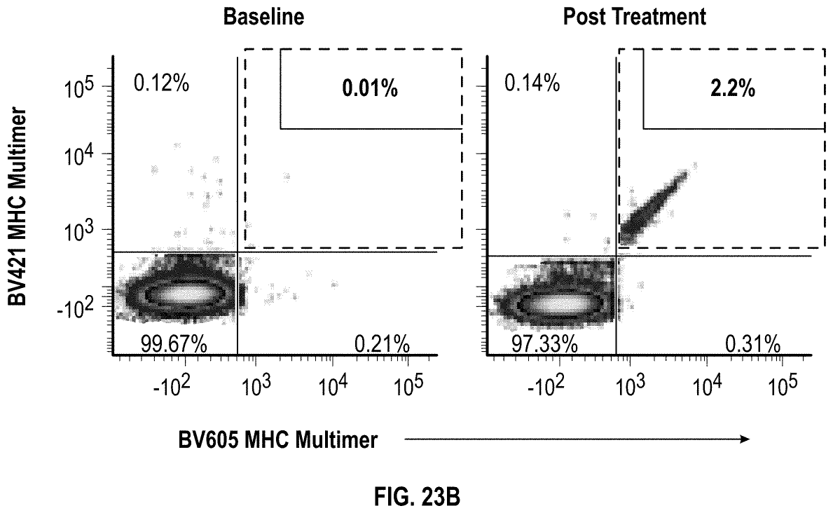

Another patient with triple negative breast cancer (indicated by the box in FIG. 22) treated with the RNA vaccine at a dose of 38 μg exhibited a partial response (PR). This patient had previously been treated with a checkpoint inhibitor (CPI-experienced) and had PD-L1 expression in ≥5% of tumor infiltrating immune cells or tumor cells as assessed by SP142 Ventana assay. As shown in FIG. 23A-23B, at baseline, this patient had several visible tumor masses associated with metastatic disease and was negative for CD8+ neoantigen-specific T cells (0.01%; background levels). At cycle 4, tumors had reduced in size and the patient had 2.2% CD8+ neoantigen-specific T cells.

### Clinical Activity in the Indication-Specific Expansion Phase

As described in Example 1 and as shown in FIG. 4, the Phase Ib study included an indication-specific expansion phase in which patients with specific tumor-types (either checkpoint inhibitor naïve or experienced) were treated with the RNA vaccine at a dose of 15 μg or 25 μg in combination with atezolizumab (1200 mg). A summary of baseline patient and disease characteristics for patients included in the indication-specific, checkpoint inhibitor naïve expansion phase of the Phase Ib study is provided in Table 12.

TABLE 12
Baseline patient characteristics in the
indication-specific expansion phase.

| | Median | PD-L1 | | |
|---|---|---|---|---|
| | (range) Prior | expression, n (%)^{a} | | |
| Cohort | Therapies, n | <5% | ≥5% | Missing |
| UC (n = 10) | 1 (1-3) | 7 (70) | 3 (30) | 0 |
| NSCLC | 1.5 (1-5) | 8 (100) | 0 | 2 |
| (n = 10) | | | | |
| TNBC (n = 22) | 3.5 (1-11) | 16 (80) | 4 (20) | 2 |
| RCC (n = 9) | 1 (1-1) | 7 (77.7) | 2 (22.2) | 0 |
| Melanoma | 1 (1-2) | 9 (90.0) | 0 | 1 |
| (n = 10) | | | | |

All patients were checkpoint inhibitor naïve;
^{a}PD-L1 expression on IC/TC analyzed by SP142 Ventana assay.
UC = urothelial carcinoma;
NSCLC = non-small cell lung cancer;
TNBC = triple negative breast cancer;
RCC = renal cell cancer.

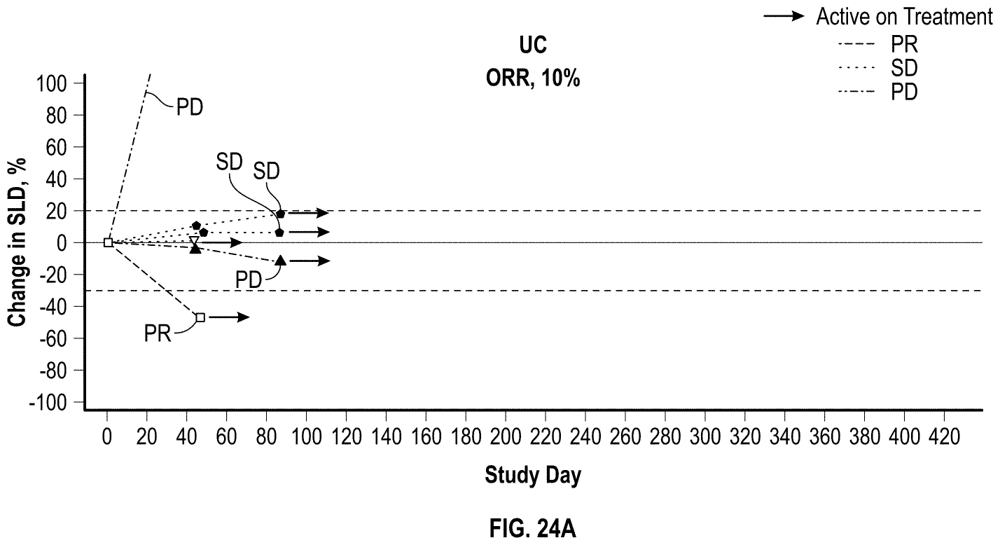

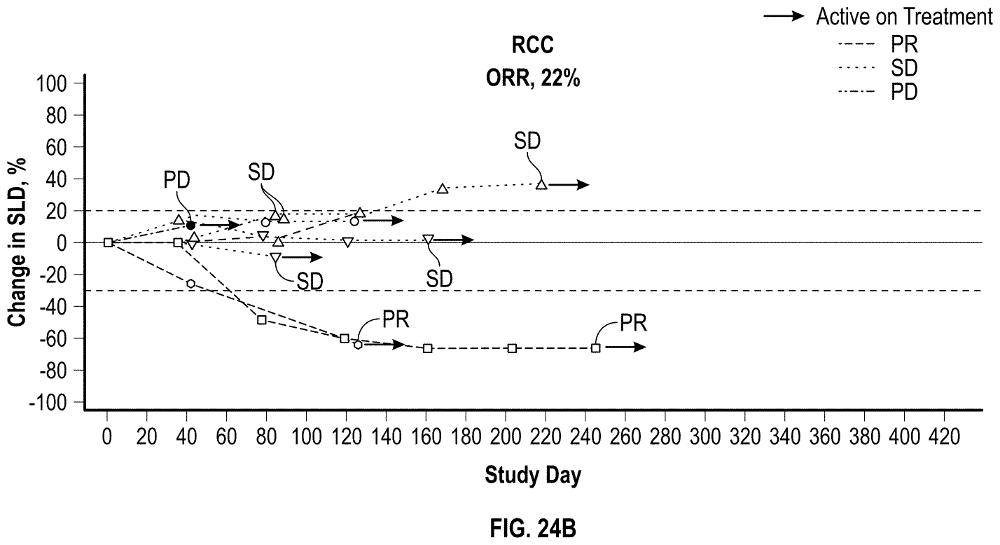

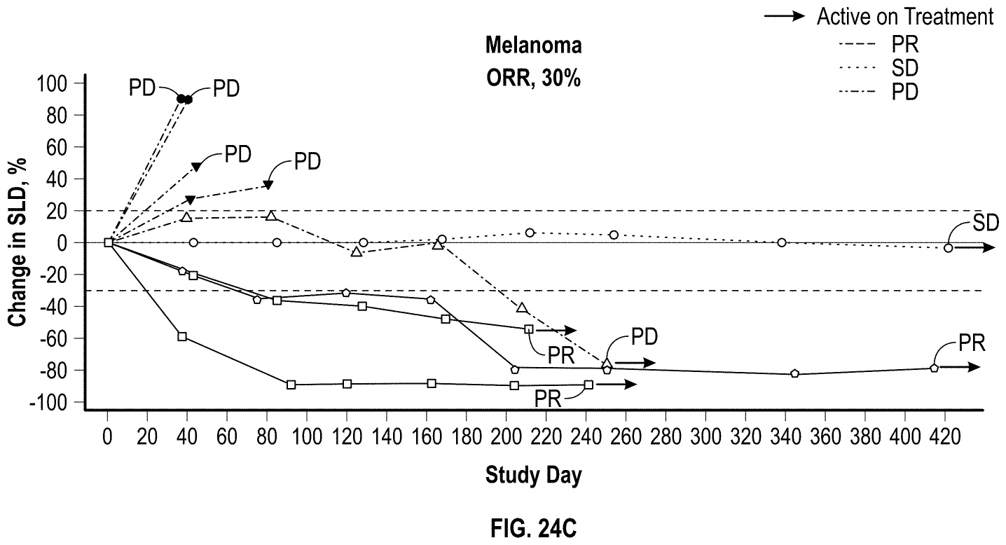

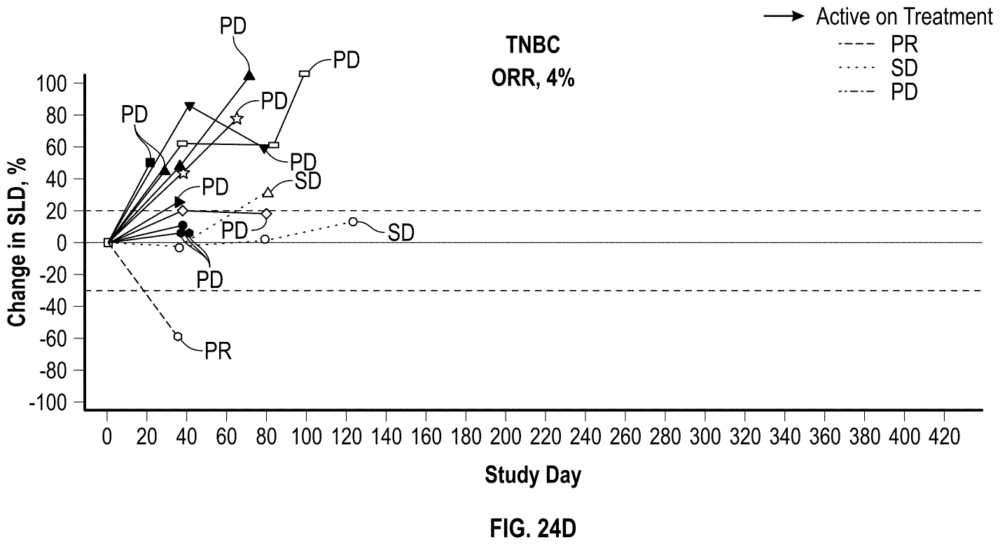

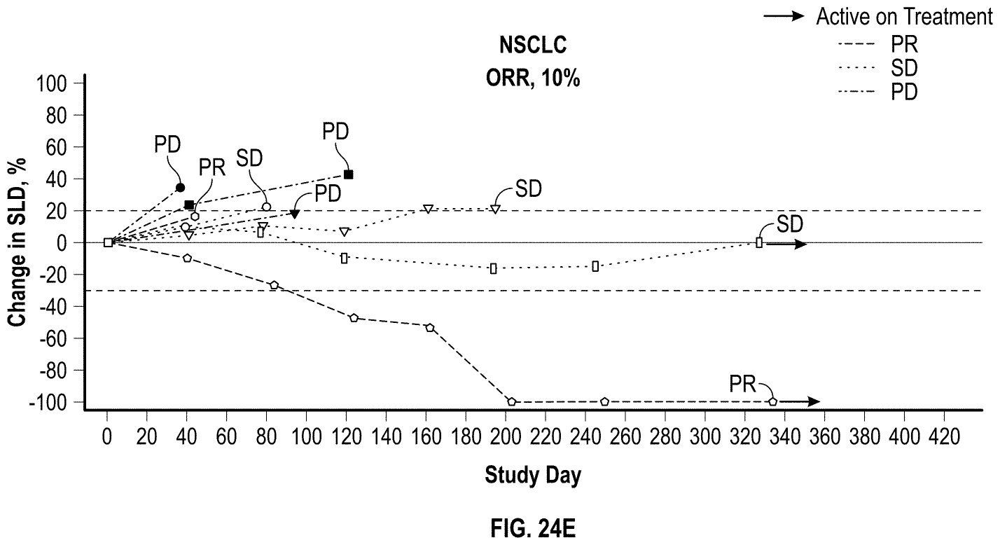

FIGS. 24A-24E provide the change overtime in the sums of longest diameters (SLDs) and objective response rates (ORRs) for checkpoint inhibitor naïve patients with urothelial carcinoma (FIG. 24A), renal cell carcinoma (FIG. 24B), melanoma (FIG. 24C), triple negative breast cancer (FIG. 24D), and non-small cell lung cancer (FIG. 24E). Urothelial carcinoma patients had an ORR of 10%, renal cell carcinoma patients had an ORR of 22%, melanoma patients had an ORR of 30%, triple negative breast cancer patients had an ORR of 4%, and non-small cell lung cancer patients had an ORR of 10%.

### Discussion

The results described in this Example showed that the RNA vaccine administered in combination with atezolizumab was generally well tolerated. No dose-limiting toxicities were observed and the maximum tolerated dose was not reached. Immune monitoring during treatment showed that administration of the RNA vaccine in combination with atezolizumab induced release of pro-inflammatory cytokines, peripheral T-cell responses in the majority of patients, and infiltration of RNA vaccine-induced T cells into the tumor of one patient. In addition, a complete response following treatment with the RNA vaccine in combination with atezolizumab was observed in one patient and objective responses were observed in several patients with various tumor types. Overall, these results are consistent with the proposed dual mechanism of action of the RNA vaccine as a stimulator of innate immune responses and neoantigen presentation (see, e.g., FIGS. 10A-10B).

SEQUENCES
All polynucleotide sequences are depicted in the 5′→3′ direction.
All polypeptide sequences are depicted in the N-terminal to
C-terminal direction.
Anti-PDL1 antibody HVR-H1 sequence
(SEQ ID NO: 1)
GFTFSDSWIH
Anti-PDL1 antibody HVR-H2 sequence
(SEQ ID NO: 2)
AWISPYGGSTYYADSVKG
Anti-PDL1 antibody HVR-H3 sequence
(SEQ ID NO: 3)
RHWPGGFDY
Anti-PDL1 antibody HVR-L1 sequence
(SEQ ID NO: 4)
RASQDVSTAVA
Anti-PDL1 antibody HVR-L2 sequence
(SEQ ID NO: 5)
SASFLYS
Anti-PDL1 antibody HVR-L3 sequence
(SEQ ID NO: 6)
QQYLYHPAT
Anti-PDL1 antibody VH sequence
(SEQ ID NO: 7)
EVQLVESGGGLVQPGGSLRLSCAASGFTFSDSWIHWVRQAPGKGLEWVAWISPYGGSTYYA
DSVKGRFTISADTSKNTAYLQMNSLRAEDTAVYYCARRHWPGGFDYWGQGTLVTVSS
Anti-PDL1 antibody VL sequence
(SEQ ID NO: 8)
DIQMTQSPSSLSASVGDRVTITCRASQDVSTAVAWYQQKPGKAPKLLIYSASF
LYSGVPSRFSGSGSGTDFTLTISSLQPEDFATYYCQQYLYHPATFGQGTKVEIKR
Anti-PDL1 antibody heavy chain sequence
(SEQ ID NO: 9)
EVQLVESGGGLVQPGGSLRLSCAASGFTFSDSWIHWVRQAPGKGLEWVAWISPYGGSTYYA
DSVKGRFTISADTSKNTAYLQMNSLRAEDTAVYYCARRHWPGGFDYWGQGTLVTVSSAST
KGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYASTYRVVSVLTV
LHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEAL
HNHYTQKSLSLSPG
Anti-PDL1 antibody light chain sequence
(SEQ ID NO: 10)
DIQMTQSPSSLSASVGDRVTITCRASQDVSTAVAWYQQKPGKAPKLLIYSASFLYSGVPSRFS
GSGSGTDFTLTISSLQPEDFATYYCQQYLYHPATFGQGTKVEIKRTVAAPSVFIFPPSDEQLKS
GTASVVCLLNNFYPREAKVQWKVDNALQSGNSQESVTEQDSKDSTYSLSSTLTLSKADYEK
HKVYACEVTHQGLSSPVTKSFNRGEC
Nivolumab heavy chain sequence
(SEQ ID NO: 11)
QVQLVESGGGVVQPGRSLRLDCKASGITFSNSGMHWVRQAPGKGLEWVAVIWY
DGSKRYYADSVKGRFTISRDNSKNTLFLQMNSLRAEDTAVYYCATNDDYWGQGTLVTVSS
ASTKGPSVFPLAPCSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEGNVFSCSVMHEAL
HNHYTQKSLSLSLG
Nivolumab light chain sequence
(SEQ ID NO: 12)
EIVLTQSPATLSLSPGERATLSCRASQSVSSYLAWYQQKPGQAPRLLIYDASNRAT
GIPARFSGSGSGTDFTLTISSLEPEDFAVYYCQQSSNWPRTFGQGTKVEIKRTVAAPSVFIFPPS
DEQLKSGTASVVCLLNNFYPREAKVQWKVDNALQSGNSQESVTEQDSKDSTYSLSSTLTLSK
ADYEKHKVYACEVTHQGLSSPVTKSFNRGEC
Pembrolizumab heavy chain sequence
(SEQ ID NO: 13)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRDYRFDMGFDYW
GQGTTVTVSSASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVESKYGPPCPPCP
APEFLGGPSVFLFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
Pembrolizumab light chain sequence
(SEQ ID NO: 14)
EIVLTQSPAT LSLSPGERATLSCRASKGVSTSGYSYLHWYQQKPGQAPRLLIYLASYLES
GVPARFSGSGSGTDFTLTISSLEPEDFAVYYCQHSRDLPLTFGGGTKVEIKRTVAAPSVF
IFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
Avelumab heavy chain sequence
(SEQ ID NO: 15)
EVQLLESGGGLVQPGGSLRLSCAASGFTFSSYIMMWVRQAPGKGLEWVSSIYPSGGITFYAD
TVKGRFTISRDNSKNTLYLQMNSLRAEDTAVYYCARIKLGTVTTVDYWGQGTLVTVSSAST
KGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEAL
HNHYTQKSLSLSPG
Avelumab light chain sequence
(SEQ ID NO: 16)
QSALTQPASVSGSPGQSITISCTGTSSDVGGYNYVSWYQQHPGKAPKLMIYDVSNRPSGVSN
RFSGSKSGNTASLTISGLQAEDEADYYCSSYTSSSTRVFGTGTKVTVLGQPKANPTVTLFPPSS
EELQANKATLVCLISDFYPGAVTVAWKADGSPVKAGVETTKPSKQSNNKYAASSYLSLTPEQ
WKSHRSYSCQVTHEGSTVEKTVAPTECS
Durvalumab heavy chain sequence
(SEQ ID NO: 17)
EVQLVESGGGLVQPGGSLRLSCAASGFTFSRYWMSWVRQAPGKGLEWVANIKQDGSEKYY
VDSVKGRFTISRDNAKNSLYLQMNSLRAEDTAVYYCAREGGWFGELAFDYWGQGTLVTVS
SASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPEFEGGPSVFLF
PPKPKDTLMISRTPEVTCVVVDVSHEDPEVKFNWYVDGVEVHNAKTKPREEQYNSTYRVVS
VLTVLHQDWLNGKEYKCKVSNKALPASIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSV
MHEALHNHYTQKSLSLSPG
Durvalumab light chain sequence
(SEQ ID NO: 18)
EIVLTQSPGTLSLSPGERATLSCRASQRVSSSYLAWYQQKPGQAPRLLIYDASSRATGIPDRFS
GSGSGTDFTLTISRLEPEDFAVYYCQQYGSLPWTFGQGTKVEIKRTVAAPSVFIFPPSDEQLKS
GTASVVCLLNNFYPREAKVQWKVDNALQSGNSQESVTEQDSKDSTYSLSSTLTLSKADYEK
HKVYACEVTHQGLSSPVTKSFNRGEC
Full PCV RNA 5′ constant sequence
(SEQ ID NO: 19)
GGCGAACUAGUAUUCUUCUGGUCCCCACAGACUCAGAGAGAACCCGCCACCAUGAGAG
UGAUGGCCCCCAGAACCCUGAUCCUGCUGCUGUCUGGCGCCCUGGCCCUGACAGAGAC
AUGGGCCGGAAGC
Full PCV RNA 3′ constant sequence
(SEQ ID NO: 20)
AUCGUGGGAAUUGUGGCAGGACUGGCAGUGCUGGCCGUGGUGGUGAUCGGAGCCGUG
GUGGCUACCGUGAUGUGCAGACGGAAGUCCAGCGGAGGCAAGGGCGGCAGCUACAGC
CAGGCCGCCAGCUCUGAUAGCGCCCAGGGCAGCGACGUGUCACUGACAGCCUAGUAAC
UCGAGCUGGUACUGCAUGCACGCAAUGCUAGCUGCCCCUUUCCCGUCCUGGGUACCCC
GAGUCUCCCCCGACCUCGGGUCCCAGGUAUGCUCCCACCUCCACCUGCCCCACUCACCA
CCUCUGCUAGUUCCAGACACCUCCCAAGCACGCAGCAAUGCAGCUCAAAACGCUUAGC
CUAGCCACACCCCCACGGGAAACAGCAGUGAUUAACCUUUAGCAAUAAACGAAAGUUU
AACUAAGCUAUACUAACCCCAGGGUUGGUCAAUUUCGUGCCAGCCACACCGAGACCUG
GUCCAGAGUCGCUAGCCGCGUCGCU
Full PCV Kozak RNA
(SEQ ID NO: 21)
GGCGAACUAGUAUUCUUCUGGUCCCCACAGACUCAGAGAGAACCCGCCACC
Full PCV Kozak DNA
(SEQ ID NO: 22)
GGCGAACTAGTATTCTTCTGGTCCCCACAGACTCAGAGAGAACCCGCCACC
short Kozak RNA
(SEQ ID NO: 23)
UUCUUCUGGUCCCCACAGACUCAGAGAGAACCCGCCACC
short Kozak DNA
(SEQ ID NO: 24)
TTCTTCTGGTCCCCACAGACTCAGAGAGAACCCGCCACC
sec RNA
(SEQ ID NO: 25)
AUGAGAGUGAUGGCCCCCAGAACCCUGAUCCUGCUGCUGUCUGGCGCCCUGGCCCUGA
CAGAGACAUGGGCCGGAAGC
sec DNA
(SEQ ID NO: 26)
ATGAGAGTGATGGCCCCCAGAACCCTGATCCTGCTGCTGTCTGGCGCCCTGGCCCTGACA
GAGACATGGGCCGGAAGC
sec protein
(SEQ ID NO: 27)
MRVMAPRTLILLLSGALALTETWAGS
MITD RNA
(SEQ ID NO: 28)
AUCGUGGGAAUUGUGGCAGGACUGGCAGUGCUGGCCGUGGUGGUGAUCGGAGCCGUG
GUGGCUACCGUGAUGUGCAGACGGAAGUCCAGCGGAGGCAAGGGCGGCAGCUACAGC
CAGGCCGCCAGCUCUGAUAGCGCCCAGGGCAGCGACGUGUCACUGACAGCC
MITD DNA
(SEQ ID NO: 29)
ATCGTGGGAATTGTGGCAGGACTGGCAGTGCTGGCCGTGGTGGTGATCGGAGCCGTGGT
GGCTACCGTGATGTGCAGACGGAAGTCCAGCGGAGGCAAGGGCGGCAGCTACAGCCAG
GCCGCCAGCTCTGATAGCGCCCAGGGCAGCGACGTGTCACTGACAGCC
MITD protein
(SEQ ID NO: 30)
IVGIVAGLAVLAVVVIGAVVATVMCRRKSSGGKGGSYSQAASSDSAQGSDVSLTA
Full PCV FI RNA
(SEQ ID NO: 31)
CUCGAGCUGGUACUGCAUGCACGCAAUGCUAGCUGCCCCUUUCCCGUCCUGGGUACCC
CGAGUCUCCCCCGACCUCGGGUCCCAGGUAUGCUCCCACCUCCACCUGCCCCACUCACC
ACCUCUGCUAGUUCCAGACACCUCCCAAGCACGCAGCAAUGCAGCUCAAAACGCUUAG
CCUAGCCACACCCCCACGGGAAACAGCAGUGAUUAACCUUUAGCAAUAAACGAAAGUU
UAACUAAGCUAUACUAACCCCAGGGUUGGUCAAUUUCGUGCCAGCCACACCGAGACCU
GGUCCAGAGUCGCUAGCCGCGUCGCU
Full PCV FI DNA
(SEQ ID NO: 32)
CTGGTACTGCATGCACGCAATGCTAGCTGCCCCTTTCCCGTCCTGGGTACCCCGAGTCTC
CCCCGACCTCGGGTCCCAGGTATGCTCCCACCTCCACCTGCCCCACTCACCACCTCTGCT
AGTTCCAGACACCTCCCAAGCACGCAGCAATGCAGCTCAAAACGCTTAGCCTAGCCACA
CCCCCACGGGAAACAGCAGTGATTAACCTTTAGCAATAAACGAAAGTTTAACTAAGCTA
TACTAACCCCAGGGTTGGTCAATTTCGTGCCAGCCACACCGAGACCTGGTCCAGAGTCGC
TAGCCGCGTCGCT
F element RNA
(SEQ ID NO: 33)
CUGGUACUGCAUGCACGCAAUGCUAGCUGCCCCUUUCCCGUCCUGGGUACCCCGAGUC
UCCCCCGACCUCGGGUCCCAGGUAUGCUCCCACCUCCACCUGCCCCACUCACCACCUCU
GCUAGUUCCAGACACCUCC
F element DNA
(SEQ ID NO: 34)
CTGGTACTGCATGCACGCAATGCTAGCTGCCCCTTTCCCGTCCTGGGTACCCCGAGTCTC
CCCCGACCTCGGGTCCCAGGTATGCTCCCACCTCCACCTGCCCCACTCACCACCTCTGCT
AGTTCCAGACACCTCC
I element RNA
(SEQ ID NO: 35)
CAAGCACGCAGCAAUGCAGCUCAAAACGCUUAGCCUAGCCACACCCCCACGGGAAACA
GCAGUGAUUAACCUUUAGCAAUAAACGAAAGUUUAACUAAGCUAUACUAACCCCAGG
GUUGGUCAAUUUCGUGCCAGCCACACCG
I element DNA
(SEQ ID NO: 36)
CAAGCACGCAGCAATGCAGCTCAAAACGCTTAGCCTAGCCACACCCCCACGGGAAACAG
CAGTGATTAACCTTTAGCAATAAACGAAAGTTTAACTAAGCTATACTAACCCCAGGGTTG
GTCAATTTCGTGCCAGCCACACCG
linker RNA
(SEQ ID NO: 37)
GGCGGCUCUGGAGGAGGCGGCUCCGGAGGC
linker DNA
(SEQ ID NO: 38)
GGCGGCTCTGGAGGAGGCGGCTCCGGAGGC
linker protein
(SEQ ID NO: 39)
GGSGGGGSGG
Full PCV DNA 5′ constant sequence
(SEQ ID NO: 40)
GGCGAACTAGTATTCTTCTGGTCCCCACAGACTCAGAGAGAACCCGCCACCATGAGAGT
GATGGCCCCCAGAACCCTGATCCTGCTGCTGTCTGGCGCCCTGGCCCTGACAGAGACATG
GGCCGGAAGC
Full PCV DNA 3′ constant sequence
(SEQ ID NO: 41)
ATCGTGGGAATTGTGGCAGGACTGGCAGTGCTGGCCGTGGTGGTGATCGGAGCCGTGGT
GGCTACCGTGATGTGCAGACGGAAGTCCAGCGGAGGCAAGGGCGGCAGCTACAGCCAG
GCCGCCAGCTCTGATAGCGCCCAGGGCAGCGACGTGTCACTGACAGCCTAGTAACTCGA
GCTGGTACTGCATGCACGCAATGCTAGCTGCCCCTTTCCCGTCCTGGGTACCCCGAGTCT
CCCCCGACCTCGGGTCCCAGGTATGCTCCCACCTCCACCTGCCCCACTCACCACCTCTGCT
AGTTCCAGACACCTCCCAAGCACGCAGCAATGCAGCTCAAAACGCTTAGCCTAGCCACA
CCCCCACGGGAAACAGCAGTGATTAACCTTTAGCAATAAACGAAAGTTTAACTAAGCTA
TACTAACCCCAGGGTTGGTCAATTTCGTGCCAGCCACACCGAGACCTGGTCCAGAGTCGC
TAGCCGCGTCGCT
Full PCV RNA with 5′ GG from cap
(SEQ ID NO: 42)
GGGGCGAACU AGUAUUCUUC UGGUCCCCAC AGACUCAGAG AGAACCCGCC
ACCAUGAGAG UGAUGGCCCC CAGAACCCUG AUCCUGCUGC UGUCUGGCGC
CCUGGCCCUG ACAGAGACAU GGGCCGGAAG CNAUCGUGGGA AUUGUGGCAG
GACUGGCAGU GCUGGCCGUG GUGGUGAUCG GAGCCGUGGU GGCUACCGUG
AUGUGCAGAC GGAAGUCCAG CGGAGGCAAG GGCGGCAGCU ACAGCCAGGC
CGCCAGCUCU GAUAGCGCCC AGGGCAGCGA CGUGUCACUG ACAGCCUAGU
AACUCGAGCU GGUACUGCAU GCACGCAAUG CUAGCUGCCC CUUUCCCGUC
CUGGGUACCC CGAGUCUCCC CCGACCUCGG GUCCCAGGUA UGCUCCCACC
UCCACCUGCC CCACUCACCA CCUCUGCUAG UUCCAGACAC CUCCCAAGCA
CGCAGCAAUG CAGCUCAAAA CGCUUAGCCU AGCCACACCC CCACGGGAAA
CAGCAGUGAU UAACCUUUAG CAAUAAACGA AAGUUUAACU AAGCUAUACU
AACCCCAGGG UUGGUCAAUU UCGUGCCAGC CACACCGAGA CCUGGUCCAG
AGUCGCUAGC CGCGUCGCUA AAAAAAAAAA AAAAAAAAAA AAAAAAAAAA
AAAAAAAAAA AAAAAAAAAA AAAAAAAAAA AAAAAAAAAA AAAAAAAAAA
AAAAAAAAAA AAAAAAAAAA AAAAAAAAAA AAAAAAAAA

What is claimed is:

1. A method of inducing neoepitope-specific CD8+ T cells in an individual with a tumor, comprising administering to the individual an effective amount of an RNA vaccine, wherein the RNA vaccine comprises one or more polynucleotides encoding one or more neoepitopes resulting from cancer-specific somatic mutations present in a tumor specimen obtained from the individual, and wherein about 1% to about 6% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine.

2. The method of claim 1, wherein the peripheral blood sample comprises about 5% or about 6% CD8+ T cells that are specific for at least one of the neoepitopes encoded by the one or more polynucleotides of the RNA vaccine.

3. The method of claim 1 or claim 2, wherein the neoepitope-specific CD8+ T cells are detected in the peripheral blood sample by ex vivo ELISPOT or MHC multimer analysis.

4. The method of any one of claims 1-3, wherein administration of the RNA vaccine to the individual results in an induction of neoepitope-specific CD4+ T cells in the peripheral blood of the individual compared to prior to administration of the RNA vaccine, wherein the neoepitope-specific CD4+ T cells are specific for at least one of the neoepitopes encoded by the one or more polynucleotides of the RNA vaccine.

5. The method of claim 4, wherein the neoepitope-specific CD4+ T cells are detected in a peripheral blood sample obtained from the individual by ex vivo ELISPOT analysis.

6. The method of any one of claims 1-5, wherein administration of the RNA vaccine to a plurality of individuals results in an induction of neoepitope-specific CD4+ or CD8+ T cells in the peripheral blood of at least about 70% of the individuals in the plurality compared to prior to administration of the RNA vaccine, wherein the neoepitope-specific CD4+ or CD8+ T cells are specific for at least one of the neoepitopes encoded by the one or more polynucleotides of the RNA vaccine, and wherein the induction of neoepitope-specific CD4+ or CD8+ T cells is assessed by ex vivo ELISPOT or MHC multimer analysis.

7. The method of any one of claims 1-6, wherein administration of the RNA vaccine to the individual results in an increase in the level of one or more inflammatory cytokines in the peripheral blood of the individual compared to the level of the one or more inflammatory cytokines prior to administration of the RNA vaccine.

8. The method of claim 7, wherein the increase in the level of the one or more inflammatory cytokines is present in the peripheral blood of the individual at between about 4 to about 6 hours after administration of the RNA vaccine.

9. The method of claim 7 or claim 8, wherein the one or more inflammatory cytokines are selected from the group consisting of IFNγ, IFNα, IL-12, and IL-6.

10. A method of inducing trafficking of neoepitope-specific CD8+ T cells to a tumor in an individual, comprising administering to the individual an effective amount of an RNA vaccine, wherein the RNA vaccine comprises one or more polynucleotides encoding one or more neoepitopes resulting from cancer-specific somatic mutations present in a tumor specimen obtained from the individual, and wherein the neoepitope-specific CD8+ T cells trafficked to the tumor after administration of the RNA vaccine are specific for at least one of the neoepitopes encoded by the one or more polynucleotides of the RNA vaccine.

11. The method of any one of claims 1-10, wherein the neoepitope-specific CD8+ T cells have a memory phenotype.

12. The method of claim 11, wherein the neoepitope-specific CD8+ T cells having a memory phenotype are effector memory T cells (Tₑₘ).

13. The method of claim 12, wherein the effector memory T cells (Tₑₘ) are CD45RO positive and CCR7 negative.

14. The method of any one of claims 1-13, wherein the neoepitope-specific CD8+ T cells are PD-1+.

15. The method of any one of claims 1-14, wherein the individual has a tumor with a low to intermediate mutational burden.

16. The method of any one of claims 1-15, wherein the individual has a low tumor burden.

17. The method of any one of claims 1-16, wherein the tumor has low or negative PD-L1 expression.

18. The method of claim 17, wherein less than 5% of tumor cells in a sample obtained from the tumor express PD-L1.

19. The method of claim 17, wherein less than 5% of immune cells in a sample obtained from the tumor express PD-L1.

20. The method of claim 18 or claim 19, wherein the percentage of tumor cells or immune cells in a sample obtained from the tumor that express PD-L1 is determined using immunohistochemistry.

21. The method of any one of claims 1-20, wherein administration of the RNA vaccine results in a complete response (CR) or partial response (PR) in the individual.

22. The method of any one of claims 1-21, wherein the individual has a locally advanced or metastatic solid tumor or has one or more metastatic relapses.

23. The method of any one of claims 1-22, wherein the tumor is a non-small cell lung (NSCLC), bladder, renal, head and neck, sarcoma, breast, melanoma, prostate, ovarian, gastric, liver, urothelial, colon, kidney, cervix, Merkel cell (MCC), endometrial, soft tissue sarcoma, esophageal, esophagogastric junction, bone sarcoma, thyroid, or colorectal tumor.

24. The method of claim 23, wherein the breast tumor is a triple-negative breast (TNBC) tumor.

25. The method of claim 23, wherein the tumor is a urothelial tumor, and wherein administration of the RNA vaccine to a plurality of individuals results in an objective response in at least about 10% of the individuals in the plurality.

26. The method of claim 23, wherein the tumor is a renal tumor, and wherein administration of the RNA vaccine to a plurality of individuals results in an objective response in at least about 22% of the individuals in the plurality.

27. The method of claim 23, wherein the tumor is a melanoma tumor, and wherein administration of the RNA vaccine to a plurality of individuals results in an objective response in at least about 30% of the individuals in the plurality.

28. The method of claim 24, wherein the tumor is a TNBC tumor, and wherein administration of the RNA vaccine to a plurality of individuals results in an objective response in at least about 4% of the individuals in the plurality.

29. The method of claim 23, wherein the tumor is an NSCLC tumor, and wherein administration of the RNA vaccine to a plurality of individuals results in an objective response in at least about 10% of the individuals in the plurality.

30. The method of any one of claims 1-29, wherein, prior to administration of the RNA vaccine, the individual has been treated with one or more cancer therapies or between 3 and 5 cancer therapies.

31. The method of any one of claims 1-29, wherein, prior to administration of the RNA vaccine, the individual has been treated with between about 1 to about 17 or between about 1 to about 9 prior systemic cancer therapies.

32. The method of any one of claims 1-31, wherein, prior to administration of the RNA vaccine, the individual has been treated with a checkpoint inhibitor therapy.

33. The method of any one of claims 1-31, wherein, prior to administration of the RNA vaccine, the individual has not been treated with a checkpoint inhibitor therapy.

34. The method of any one of claims 1-33, wherein the RNA vaccine comprises one or more polynucleotides encoding 10-20 neoepitopes resulting from cancer-specific somatic mutations present in the tumor specimen.

35. The method of any one of claims 1-34, wherein the RNA vaccine is formulated in a lipoplex nanoparticle or liposome.

36. The method of claim 35, wherein the lipoplex nanoparticle or liposome comprises one or more lipids that form a multilamellar structure that encapsulates the RNA of the RNA vaccine.

37. The method of claim 36, wherein the one or more lipids comprises at least one cationic lipid and at least one helper lipid.

38. The method of claim 36, wherein the one or more lipids comprises (R)-N,N,N-trimethyl-2,3-dioleyloxy-1-propanaminium chloride (DOTMA) and 1,2-dioleoyl-sn-glycero-3-phosphoethanolamine (DOPE).

39. The method of claim 38, wherein at physiological pH the overall charge ratio of positive charges to negative charges of the liposome is 1.3:2 (0.65).

40. The method of any one of claims 1-39, wherein the RNA vaccine is administered to the individual at a dose of about 15 μg, about 25 μg, about 38 μg, about 50 μg, about 75 μg, or about 100 μg.

41. The method of any one of claims 1-40, wherein the RNA vaccine is administered intravenously to the individual.

42. The method of any one of claims 1-41, wherein the RNA vaccine is administered to the individual at an interval of 7 days or 1 week.

43. The method of any one of claims 1-41, wherein the RNA vaccine is administered to the individual at an interval of 14 days or 2 weeks.

44. The method of claim 42 or claim 43, wherein the RNA vaccine is administered to the individual for 12 weeks or 84 days.

45. The method of any one of claims 1-41, wherein the RNA vaccine is administered to the individual in 21-day Cycles, wherein the RNA vaccine is administered to the individual on Days 1, 8, and 15 of Cycle 1; Days 1, 8, and 15 of Cycle 2; Days 1 and 15 of Cycle 3; and Day 1 of Cycle 7.

46. The method of claim 45, further comprising administering the RNA vaccine on Day 1 of Cycle 13, and every 24 weeks or 168 days thereafter.

47. The method of claim 46, wherein administration of the RNA vaccine continues until an occurrence of disease progression in the individual.

48. The method of any one of claims 1-41, wherein the RNA vaccine is administered to the individual in an induction stage and a maintenance stage after the induction stage, wherein the RNA vaccine is administered to the individual during the induction stage at an interval of 1 or 2 weeks, and wherein the RNA vaccine is administered to the individual during the maintenance stage at an interval of 24 weeks.

49. The method of any one of claims 1-41, wherein the RNA vaccine is administered to the individual in an induction stage and a maintenance stage after the induction stage, wherein the RNA vaccine is administered to the individual during the induction stage at an interval of 7 or 14 days, and wherein the RNA vaccine is administered to the individual during the maintenance stage at an interval of 168 days.

50. The method of any one of claims 1-41, wherein the RNA vaccine is administered to the individual in an induction stage and a maintenance stage after the induction stage, wherein the RNA vaccine is administered to the individual in 21-day Cycles;
wherein, during the induction stage, the RNA vaccine is administered to the individual on Days 1, 8, and 15 of Cycle 1; Days 1, 8, and 15 of Cycle 2; Days 1 and 15 of Cycle 3; and Day 1 of Cycle 7; and
wherein, during the maintenance stage, the RNA vaccine is administered to the individual on Day 1 of Cycle 13 and once every 24 weeks or 168 days thereafter.

51. The method of claim 48 or claim 49, wherein the induction stage comprises up to 9 administrations of the RNA vaccine.

52. The method of any one of claims 48-51, wherein the maintenance stage continues until an occurrence of disease progression in the individual.

53. The method of any one of claims 1-52, wherein the RNA vaccine comprises an RNA molecule comprising, in the 5′→3′ direction:
(1) a 5′ cap;
(2) a 5′ untranslated region (UTR);
(3) a polynucleotide sequence encoding a secretory signal peptide;
(4) a polynucleotide sequence encoding the one or more neoepitopes resulting from cancer-specific somatic mutations present in the tumor specimen;
(5) a polynucleotide sequence encoding at least a portion of a transmembrane and cytoplasmic domain of a major histocompatibility complex (MHC) molecule;
(6) a 3′ UTR comprising:
(a) a 3′ untranslated region of an Amino-Terminal Enhancer of Split (AES) mRNA or a fragment thereof; and
(b) non-coding RNA of a mitochondrially encoded 12S RNA or a fragment thereof; and
(7) a poly(A) sequence.

54. The method of claim 53, wherein the RNA molecule further comprises a polynucleotide sequence encoding an amino acid linker; wherein the polynucleotide sequences encoding the amino acid linker and a first of the one or more neoepitopes form a first linker-neoepitope module; and wherein the polynucleotide sequences forming the first linker-neoepitope module are between the polynucleotide sequence encoding the secretory signal peptide and the polynucleotide sequence encoding the at least portion of the transmembrane and cytoplasmic domain of the MHC molecule in the 5′→3′ direction.

55. The method of claim 54, wherein the amino acid linker comprises the sequence GGSGGGGSGG (SEQ ID NO:39).

56. The method of claim 54, wherein the polynucleotide sequence encoding the amino acid linker comprises the sequence GGCGGCUCUGGAGGAGGCGGCUCCGGAGGC (SEQ ID NO:37).

57. The method of any one of claims 54-56, wherein the RNA molecule further comprises, in the 5′→3′ direction: at least a second linker-epitope module, wherein the at least second linker-epitope module comprises a polynucleotide sequence encoding an amino acid linker and a polynucleotide sequence encoding a neoepitope; wherein the polynucleotide sequences forming the second linker-neoepitope module are between the polynucleotide sequence encoding the neoepitope of the first linker-neoepitope module and the polynucleotide sequence encoding the at least portion of the transmembrane and cytoplasmic domain of the MHC molecule in the 5′→3′ direction; and wherein the neoepitope of the first linker-epitope module is different from the neoepitope of the second linker-epitope module.

58. The method of claim 57, wherein the RNA molecule comprises 5 linker-epitope modules, and wherein the 5 linker-epitope modules each encode a different neoepitope.

59. The method of claim 57, wherein the RNA molecule comprises 10 linker-epitope modules, and wherein the 10 linker-epitope modules each encode a different neoepitope.

60. The method of claim 57, wherein the RNA molecule comprises 20 linker-epitope modules, and wherein the 20 linker-epitope modules each encode a different neoepitope.

61. The method of any one of claims 53-60, wherein the RNA molecule further comprises a second polynucleotide sequence encoding an amino acid linker, wherein the second polynucleotide sequence encoding the amino acid linker is between the polynucleotide sequence encoding the neoepitope that is most distal in the 3′ direction and the polynucleotide sequence encoding the at least portion of the transmembrane and cytoplasmic domain of the MHC molecule.

62. The method of any one of claims 53-61, wherein the 5′ cap comprises a D1 diastereoisomer of the structure:

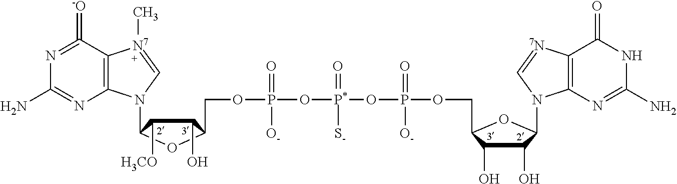

63. The method of any one of claims 53-62, wherein the 5′ UTR comprises the sequence
(SEQ ID NO: 23)
UUCUUCUGGUCCCCACAGACUCAGAGAGAACCCGCCACC

64. The method of any one of claims 53-62, wherein the 5′ UTR comprises the sequence
(SEQ ID NO: 21)
GGCGAACUAGUAUUCUUCUGGUCCCCACAGACUCAGAGAGAACCCGCC
ACC.

65. The method of any one of claims 53-64, wherein the secretory signal peptide comprises the amino acid sequence MRVMAPRTLILLLSGALALTETWAGS (SEQ ID NO:27).

66. The method of any one of claims 53-64, wherein the polynucleotide sequence encoding the secretory signal peptide comprises the sequence
(SEQ ID NO: 25)
AUGAGAGUGAUGGCCCCCAGAACCCUGAUCCUGCUGCUGUCUGGCGCC
CUGGCCCUGACAGAGACAUGGGCCGGAAGC.

67. The method of any one of claims 53-66, wherein the at least portion of the transmembrane and cytoplasmic domain of the MHC molecule comprises the amino acid sequence
(SEQ ID NO: 30)
IVGIVAGLAVLAVVVIGAVVATVMCRRKSSGGKGGSYSQAASSDSAQGS
DVSLTA.

68. The method of any one of claims 53-66, wherein the polynucleotide sequence encoding the at least portion of the transmembrane and cytoplasmic domain of the MHC molecule comprises the sequence
(SEQ ID NO: 28)
AUCGUGGGAAUUGUGGCAGGACUGGCAGUGCUGGCCGUGGUGGUGAUCG
GAGCCGUGGUGGCUACCGUGAUGUGCAGACGGAAGUCCAGCGGAGGCAA
GGGCGGCAGCUACAGCCAGGCCGCCAGCUCUGAUAGCGCCCAGGGCAGC
GACGUGUCACUGACAGCC.

69. The method of any one of claims 53-68, wherein the 3′ untranslated region of the AES mRNA comprises the sequence
(SEQ ID NO: 33)
CUGGUACUGCAUGCACGCAAUGCUAGCUGCCCCUUUCCCGUCCUGGGUA
CCCCGAGUCUCCCCCGACCUCGGGUCCCAGGUAUGCUCCCACCUCCACC
UGCCCCACUCACCACCUCUGCUAGUUCCAGACACCUCC.

70. The method of any one of claims 53-69, wherein the non-coding RNA of the mitochondrially encoded 12S RNA comprises the sequence
(SEQ ID NO: 35)
CAAGCACGCAGCAAUGCAGCUCAAAACGCUUAGCCUAGCCACACCCCCA
CGGGAAACAGCAGUGAUUAACCUUUAGCAAUAAACGAAAGUUUAACUAA
GCUAUACUAACCCCAGGGUUGGUCAAUUUCGUGCCAGCCACACCG.

71. The method of any one of claims 53-70, wherein the 3′ UTR comprises the sequence
(SEQ ID NO: 31)
CUCGAGCUGGUACUGCAUGCACGCAAUGCUAGCUGCCCCUUUCCCGUCC
UGGGUACCCCGAGUCUCCCCCGACCUCGGGUCCCAGGUAUGCUCCCACC
UCCACCUGCCCCACUCACCACCUCUGCUAGUUCCAGACACCUCCCAAGC
ACGCAGCAAUGCAGCUCAAAACGCUUAGCCUAGCCACACCCCCACGGGA
AACAGCAGUGAUUAACCUUUAGCAAUAAACGAAAGUUUAACUAAGCUAU
ACUAACCCCAGGGUUGGUCAAUUUCGUGCCAGCCACACCGAGACCUGGU
CCAGAGUCGCUAGCCGCGUCGCU.

72. The method of any one of claims 53-71, wherein the poly(A) sequence comprises 120 adenine nucleotides.

73. The method of any one of claims 1-52, wherein the RNA vaccine comprises an RNA molecule comprising, in the 5′→3′ direction:
the polynucleotide sequence
(SEQ ID NO: 19)
GGCGAACUAGUAUUCUUCUGGUCCCCACAGACUCAGAGAGAACCCGCCA
CCAUGAGAGUGAUGGCCCCCAGAACCCUGAUCCUGCUGCUGUCUGGCGC
CCUGGCCCUGACAGAGACAUGGGCCGGAAGC;
a polynucleotide sequence encoding the one or more neoepitopes resulting from cancer-specific somatic mutations present in the tumor specimen; and
the polynucleotide sequence
(SEQ ID NO: 20)
AUCGUGGGAAUUGUGGCAGGACUGGCAGUGCUGGCCGUGGUGGUGAUCG
GAGCCGUGGUGGCUACCGUGAUGUGCAGACGGAAGUCCAGCGGAGGCAA
GGGCGGCAGCUACAGCCAGGCCGCCAGCUCUGAUAGCGCCCAGGGCAGC
GACGUGUCACUGACAGCCUAGUAACUCGAGCUGGUACUGCAUGCACGCA
AUGCUAGCUGCCCCUUUCCCGUCCUGGGUACCCCGAGUCUCCCCCGACC
UCGGGUCCCAGGUAUGCUCCCACCUCCACCUGCCCCACUCACCACCUCU
GCUAGUUCCAGACACCUCCCAAGCACGCAGCAAUGCAGCUCAAAACGCU
UAGCCUAGCCACACCCCCACGGGAAACAGCAGUGAUUAACCUUUAGCAA
UAAACGAAAGUUUAACUAAGCUAUACUAACCCCAGGGUUGGUCAAUUUC
GUGCCAGCCACACCGAGACCUGGUCCAGAGUCGCUAGCCGCGUCGCU.

74. The method of any one of claims 1-73, further comprising administering a PD-1 axis binding antagonist to the individual.

75. The method of claim 74, wherein the PD-1 axis binding antagonist is a PD-1 binding antagonist.

76. The method of claim 75, wherein the PD-1 binding antagonist is an anti-PD-1 antibody.

77. The method of claim 76, wherein the anti-PD-1 antibody is nivolumab or pembrolizumab.

78. The method of claim 74, wherein the PD-1 axis binding antagonist is a PD-L1 binding antagonist.

79. The method of claim 78, wherein the PD-L1 binding antagonist is an anti-PD-L1 antibody.

80. The method of claim 79, wherein the anti-PD-L1 antibody is avelumab or durvalumab.

81. The method of claim 79, wherein the anti-PD-L1 antibody comprises:
(a) a heavy chain variable region (VH) that comprises an HVR-L1 comprising an amino acid sequence of GFTFSDSWIH (SEQ ID NO:1), an HVR-2 comprising an amino acid sequence of AWISPYGGSTYYADSVKG (SEQ ID NO:2), and HVR-3 comprising an amino acid RHWPGGFDY (SEQ ID NO:3), and
(b) a light chain variable region (VL) that comprises an HVR-L1 comprising an amino acid sequence of RASQDVSTAVA (SEQ ID NO:4), an HVR-L2 comprising an amino acid sequence of SASFLYS (SEQ ID NO:5), and an HVR-L3 comprising an amino acid sequence of QQYLYHPAT (SEQ ID NO:6).

82. The method of claim 79, wherein the anti-PD-L1 antibody comprises a heavy chain variable region (V_{H}) comprising an amino acid sequence of SEQ ID NO:7 and a light chain variable region (V_{L}) comprising an amino acid sequence of SEQ ID NO:8.

83. The method of claim 79, wherein the anti-PD-L1 antibody is atezolizumab.

84. The method of any one of claims 74-83, wherein the PD-1 axis binding antagonist is administered intravenously to the individual.

85. The method of any one of claims 79-84, wherein the anti-PD-L1 antibody is administered to the individual at a dose of about 1200 mg.

86. The method of any one of claims 74-85, wherein the PD-1 axis binding antagonist is administered to the individual at an interval of 21 days or 3 weeks.

87. The method of any one of claims 83-86, wherein the atezolizumab is administered to the individual in 21-day cycles, wherein atezolizumab is administered on Day 1 of each of Cycles 1, 2, 3, 4, 5, 6, 7, 8, 9, 10, 11, and 12.

88. The method of claim 87, further comprising administering atezolizumab on Day 1 of Cycle 13, and every 3 weeks or 21 days thereafter.

89. The method of claim 88, wherein administration of atezolizumab continues until an occurrence of disease progression in the individual.

90. The method of any one of claims 83-86, wherein the atezolizumab is administered to the individual in 21-day cycles during an induction stage and during a maintenance stage after the induction stage;
wherein, during the induction stage, atezolizumab is administered on Day 1 of each of Cycles 1, 2, 3, 4, 5, 6, 7, 8, 9, 10, 11, and 12; and
wherein, during the maintenance stage after the induction stage, atezolizumab is administered on Day 1 of Cycle 13, and every 3 weeks or 21 days thereafter.

91. The method of claim 90, wherein the maintenance stage continues until an occurrence of disease progression in the individual.

92. The method of any one of claims 1-91, wherein the individual is a human.

93. An RNA vaccine for use in a method of inducing neoepitope-specific CD8+ T cells in an individual with a tumor, said method comprising administering to the individual an effective amount of the RNA vaccine, wherein the RNA vaccine comprises one or more polynucleotides encoding one or more neoepitopes resulting from cancer-specific somatic mutations present in a tumor specimen obtained from the individual, and wherein about 1% to about 6% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine.

94. An RNA vaccine for use in a method of inducing trafficking of neoepitope-specific CD8+ T cells to a tumor in an individual, said method comprising administering to the individual an effective amount of the RNA vaccine, wherein the RNA vaccine comprises one or more polynucleotides encoding one or more neoepitopes resulting from cancer-specific somatic mutations present in a tumor specimen obtained from the individual, and wherein the neoepitope-specific CD8+ T cells trafficked to the tumor after administration of the RNA vaccine are specific for at least one of the neoepitopes encoded by the one or more polynucleotides of the RNA vaccine.

95. The RNA vaccine for use of claim 93 or claim 94, wherein the method further comprises administering a PD-1 axis binding antagonist to the individual.

96. A PD-1 axis binding antagonist for use in a method of inducing neoepitope-specific CD8+ T cells in an individual with a tumor, said method comprising administering to the individual an effective amount of the PD-1 axis binding antagonist and an RNA vaccine, wherein the RNA vaccine comprises one or more polynucleotides encoding one or more neoepitopes resulting from cancer-specific somatic mutations present in a tumor specimen obtained from the individual, and wherein about 1% to about 6% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine.

97. A PD-1 axis binding antagonist for use in a method of inducing trafficking of neoepitope-specific CD8+ T cells to a tumor in an individual, said method comprising administering to the individual an effective amount of the PD-1 axis binding antagonist and an RNA vaccine, wherein the RNA vaccine comprises one or more polynucleotides encoding one or more neoepitopes resulting from cancer-specific somatic mutations present in a tumor specimen obtained from the individual, and wherein the neoepitope-specific CD8+ T cells trafficked to the tumor after administration of the PD-1 axis binding antagonist and the RNA vaccine are specific for at least one of the neoepitopes encoded by the one or more polynucleotides of the RNA vaccine.

98. A method of inducing neoepitope-specific CD8+ T cells in an individual with a tumor, comprising administering to the individual an effective amount of an RNA vaccine, wherein the RNA vaccine comprises one or more polynucleotides encoding one or more neoepitopes resulting from cancer-specific somatic mutations present in a tumor specimen obtained from the individual, and wherein at least about 1% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine.

99. An RNA vaccine for use in a method of inducing neoepitope-specific CD8+ T cells in an individual with a tumor, said method comprising administering to the individual an effective amount of the RNA vaccine, wherein the RNA vaccine comprises one or more polynucleotides encoding one or more neoepitopes resulting from cancer-specific somatic mutations present in a tumor specimen obtained from the individual, and wherein at least about 1% of CD8+ T cells in a peripheral blood sample obtained from the individual after administration of the RNA vaccine are neoepitope-specific CD8+ T cells that are specific for at least one of the neoepitopes encoded by the one or more polynucleotides of the RNA vaccine.

100. A PD-1 axis binding antagonist for use in a method of inducing neoepitope-specific CD8+ T cells in an individual with a tumor, said method comprising administering to the individual an effective amount of the PD-1 axis binding antagonist and an RNA vaccine, wherein the RNA vaccine comprises one or more polynucleotides encoding one or more neoepitopes resulting from cancer-specific somatic mutations present in a tumor specimen obtained from the individual, and wherein at least about 1% of CD8+ T cells in a peripheral blood sample obtained from the individual after administration of the PD-1 axis binding antagonist and the RNA vaccine are neoepitope-specific CD8+ T cells that are specific for at least one of the neoepitopes encoded by the one or more polynucleotides of the RNA vaccine.


SEQUENCE LISTING (42 sequences)

SEQ ID NO: 1 - protein, 10 aa, Artificial Sequence
  Synthetic Construct
GFTFSDSWIH

SEQ ID NO: 2 - protein, 18 aa, Artificial Sequence
  Synthetic Construct
AWISPYGGSTYYADSVKG

SEQ ID NO: 3 - protein, 9 aa, Artificial Sequence
  Synthetic Construct
RHWPGGFDY

SEQ ID NO: 4 - protein, 11 aa, Artificial Sequence
  Synthetic Construct
RASQDVSTAVA

SEQ ID NO: 5 - protein, 7 aa, Artificial Sequence
  Synthetic Construct
SASFLYS

SEQ ID NO: 6 - protein, 9 aa, Artificial Sequence
  Synthetic Construct
QQYLYHPAT

SEQ ID NO: 7 - protein, 118 aa, Artificial Sequence [sequence omitted]

SEQ ID NO: 8 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 9 - protein, 447 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 439 aa, Artificial Sequence
  Synthetic Construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 214 aa, Artificial Sequence
  Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 446 aa, Artificial Sequence
  Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 14 - protein, 218 aa, Artificial Sequence
  Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 449 aa, Artificial Sequence
  Synthetic Construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 216 aa, Artificial Sequence
  Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 17 - protein, 450 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 215 aa, Artificial Sequence
  Synthetic Construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - RNA, 129 nt, Artificial Sequence
  Synthetic Construct
ggcgaacuaguauucuucugguccccacagacucagagagaacccgccaccaugagagugauggcccccagaacccugauccugcugcugucuggcgcccuggcccugacagagacaugggccggaagc

SEQ ID NO: 20 - RNA, 488 nt, Artificial Sequence
  Synthetic Construct
aucgugggaauuguggcaggacuggcagugcuggccgugguggugaucggagccgugguggcuaccgugaugugcagacggaaguccagcggaggcaagggcggcagcuacagccaggccgccagcucugauagcgcccagggcagcgacgugucacugacagccuaguaacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcu

SEQ ID NO: 21 - RNA, 51 nt, Artificial Sequence
  Synthetic Construct
ggcgaacuaguauucuucugguccccacagacucagagagaacccgccacc

SEQ ID NO: 22 - DNA, 51 bp, Artificial Sequence
  Synthetic Construct
ggcgaactagtattcttctggtccccacagactcagagagaacccgccacc

SEQ ID NO: 23 - RNA, 39 nt, Artificial Sequence
  Synthetic Construct
uucuucugguccccacagacucagagagaacccgccacc

SEQ ID NO: 24 - DNA, 39 bp, Artificial Sequence
  Synthetic Construct
ttcttctggtccccacagactcagagagaacccgccacc

SEQ ID NO: 25 - RNA, 78 nt, Artificial Sequence
  Synthetic Construct
augagagugauggcccccagaacccugauccugcugcugucuggcgcccuggcccugacagagacaugggccggaagc

SEQ ID NO: 26 - DNA, 78 bp, Artificial Sequence
  Synthetic Construct
atgagagtgatggcccccagaaccctgatcctgctgctgtctggcgccctggccctgacagagacatgggccggaagc

SEQ ID NO: 27 - protein, 26 aa, Artificial Sequence
  Synthetic Construct
MRVMAPRTLILLLSGALALTETWAGS

SEQ ID NO: 28 - RNA, 165 nt, Artificial Sequence
  Synthetic Construct
aucgugggaauuguggcaggacuggcagugcuggccgugguggugaucggagccgugguggcuaccgugaugugcagacggaaguccagcggaggcaagggcggcagcuacagccaggccgccagcucugauagcgcccagggcagcgacgugucacugacagcc

SEQ ID NO: 29 - DNA, 165 bp, Artificial Sequence
  Synthetic Construct
atcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcc

SEQ ID NO: 30 - protein, 55 aa, Artificial Sequence
  Synthetic Construct
IVGIVAGLAVLAVVVIGAVVATVMCRRKSSGGKGGSYSQAASSDSAQGSDVSLTA

SEQ ID NO: 31 - RNA, 317 nt, Artificial Sequence
  Synthetic Construct
cucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcu

SEQ ID NO: 32 - DNA, 311 bp, Artificial Sequence
  Synthetic Construct
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 33 - RNA, 136 nt, Artificial Sequence
  Synthetic Construct
cugguacugcaugcacgcaaugcuagcugccccuuucccguccuggguaccccgagucucccccgaccucgggucccagguaugcucccaccuccaccugccccacucaccaccucugcuaguuccagacaccucc

SEQ ID NO: 34 - DNA, 136 bp, Artificial Sequence
  Synthetic Construct
ctggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 35 - RNA, 143 nt, Artificial Sequence
  Synthetic Construct
caagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccg

SEQ ID NO: 36 - DNA, 143 bp, Artificial Sequence
  Synthetic Construct
caagcacgcagcaatgcagctcaaaacgcttagcctagccacacccccacgggaaacagcagtgattaacctttagcaataaacgaaagtttaactaagctatactaaccccagggttggtcaatttcgtgccagccacaccg

SEQ ID NO: 37 - RNA, 30 nt, Artificial Sequence
  Synthetic Construct
ggcggcucuggaggaggcggcuccggaggc

SEQ ID NO: 38 - DNA, 30 bp, Artificial Sequence
  Synthetic Construct
ggcggctctggaggaggcggctccggaggc

SEQ ID NO: 39 - protein, 10 aa, Artificial Sequence
  Synthetic Construct
GGSGGGGSGG

SEQ ID NO: 40 - DNA, 129 bp, Artificial Sequence
  Synthetic Construct
ggcgaactagtattcttctggtccccacagactcagagagaacccgccaccatgagagtgatggcccccagaaccctgatcctgctgctgtctggcgccctggccctgacagagacatgggccggaagc

SEQ ID NO: 41 - DNA, 488 bp, Artificial Sequence
  Synthetic Construct
atcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 42 - RNA, 740 nt, Artificial Sequence
  Synthetic Construct
  misc_feature 1,2: Linkage between the first two G residues is the unusual bond (5′->5′)-pp(s)p-, e.g., as shown in Table 1 and in FIG. 3 of the specification
  misc_feature 132: n = A,T,C, G or U
  misc_feature 132: Exists as polynucleotide sequence(s) as defined in the specification and may encode neoepitopes, optionally separated by linkers, as defined in the specification (e.g., FIGS. 1-2)
ggggcgaacuaguauucuucugguccccacagacucagagagaacccgccaccaugagagugauggcccccagaacccugauccugcugcugucuggcgcccuggcccugacagagacaugggccggaagcnaucgugggaauuguggcaggacuggcagugcuggccgugguggugaucggagccgugguggcuaccgugaugugcagacggaaguccagcggaggcaagggcggcagcuacagccaggccgccagcucugauagcgcccagggcagcgacgugucacugacagccuaguaacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa